(19) United States
(12) Patent Application Publication
(10) Pub. No.: US 2007/0037862 A1
(43) Pub. Date: Feb. 15, 2007

(54) Thiazolidinones, their production and use as pharmaceutical agents

(76) Inventors: Gerhard Siemeister, Berlin (DE); Hans Briem, Bremen (DE); Volker Schulze, Berlin (DE); Knut Eis, Berlin (DE); Lars Wortmann, Berlin (DE); Wolfgang Schwede, Glienicke (DE); Herbert Schneider, Berlin (DE); Uwe Eberspaecher, Berlin (DE); Holger Hess-Stumpp, Berlin (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/978,225

(22) Filed: Nov. 1, 2004

Related U.S. Application Data
(60) Provisional application No. 60/517,061, filed on Nov. 5, 2003.

(30) Foreign Application Priority Data
Oct. 31, 2003 (DE) 10351744.8-44

(51) Int. Cl. A61K 31/427; A61K 31/426; C07D 277/34

(52) U.S. Cl. 514/369; 548/186

(57) ABSTRACT

Thiazolidinones of general formula I 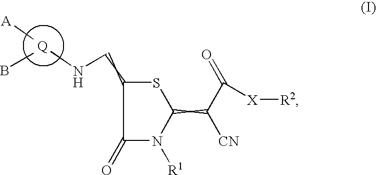
in which Q, A, B, X, R¹ and R² have the meanings that are indicated in the description, as well as those of general formula IA 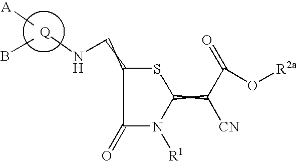
in which Q, A, B, X, R¹ and R^{2a} have the meanings that are indicated in the description, their production and use as inhibitors of the polo-like kinase (PLK) for treating various diseases as well as intermediate products for the production of thiazolidinones are described.

This application claims the benefit of the filing date of U.S. Provisional Application Ser. No. 60/517,061 filed Nov. 5, 2003 which is incorporated by reference herein.

The invention relates to thiazolidones, their production and use as inhibitors of polo-like kinase (Plk) for treating various diseases.

Tumor cells are distinguished by an uninhibited cell-cycle process. This is based on, on the one hand, the loss of control proteins, such as RB, p16, p21, p53, etc., as well as the activation of so-called accelerators of the cell-cycle process, the cyclin-dependent kinases (Cdks). The Cdks are an anti-tumor target protein that is acknowledged in pharmaceutics. In addition to the Cdks, serine/threonine kinases that regulate the new cell cycle, so-called ‘polo-like kinases,’ were described, which are involved not only in the regulation of the cell cycle but also in the coordination with other processes during mitosis and cytokinesis (formation of the spindle apparatus, chromosome separation). This class of proteins therefore represents an advantageous point of application for therapeutic intervention of proliferative diseases such as cancer (Descombes and Nigg. Embo J, 17; 1328 ff, 1998; Glover et al. Genes Dev 12, 3777 ff, 1998).

A high expression rate of Plk-1 was found in ‘non-small cell lung’ cancer (Wolf et al. Oncogene, 14, 543ff, 1997), in melanomas (Strebhardt et al. JAMA, 283, 479ff, 2000), in ‘squamous cell carcinomas’ (Knecht et al. Cancer Res, 59, 2794ff, 1999) and in ‘esophageal carcinomas’ (Tokumitsu et al. Int J Oncol 15, 687ff, 1999).

A correlation of a high expression rate in tumor patients with poor prognosis was shown for the most varied tumors (Strebhardt et al. JAMA, 283, 479ff, 2000, Knecht et al. Cancer Res, 59, 2794ff, 1999 and Tokumitsu et al. Int J Oncol 15, 687ff, 1999).

The constitutive expression of Plk-1 in NIH-3T3 cells resulted in a malignant transformation (increased proliferation, growth in soft agar, colony formation and tumor development in hairless mice (Smith et al. Biochem Biophys Res Comm, 234, 397ff., 1997).

Microinjections of Plk-1 antibodies in HeLa cells resulted in improper mitosis (Lane et al.; Journal Cell Biol, 135, 1701ff, 1996).

With a ‘20-mer’ antisense oligo, it was possible to inhibit the expression of Plk-1 in A549 cells, and to stop their ability to survive. It was also possible to show a significant anti-tumor action in hairless mice (Mundt et al., Biochem Biophys Res Comm, 269, 377ff., 2000).

The microinjection of anti-Plk antibodies in non-immortalized human Hs68 cells showed, in comparison to HeLa cells, a significantly higher fraction of cells, which remained in a growth arrest at G2 and showed far fewer signs of improper mitosis (Lane et al.; Journal Cell Biol, 135, 1701ff, 1996).

In contrast to tumor cells, antisense-oligo-molecules did not inhibit the growth and the viability of primary human mesangial cells (Mundt et al., Biochem Biophys Res Comm, 269, 377ff., 2000).

In mammals, to date in addition to the Plk-1, three other polo-kinases were described that are induced as a mitogenic response and exert their function in the G1 phase of the cell cycle. These are, on the one hand, the so-called Prk/Plk-3 (the human homologue of the mouse−Fnk=fibroblast growth factor-induced kinase; Wiest et al, Genes, Chromosomes & Cancer, 32: 384ff, 2001), Snk/Plk-2 (serum-induced kinase, Liby et al., DNA Sequence, 11, 527-33, 2001) and sak/Plk4 (Fode et al., Proc. Natl. Acad. Sci. U.S.A., 91, 6388ff; 1994).

The inhibition of Plk-1 and the other kinases of the polo family, such as Plk-2, Plk-3 and Plk-4, thus represents a promising approach for the treatment of various diseases.

The sequence identity within the Plk domains of the polo family is between 40 and 60%, so that partial interaction of inhibitors of a kinase occurs with one or more other kinases of this family. Depending on the structure of the inhibitor, however, the action can also take place selectively or preferably on only one kinase of the polo family.

In International Application WO03/093249, thiazolidinone compounds that inhibit the kinases of the polo family are disclosed.

The object of this invention consists in that additional substances that inhibit the kinases of the polo family in the nanomolar range are available.

It has now been found that compounds of general formula I 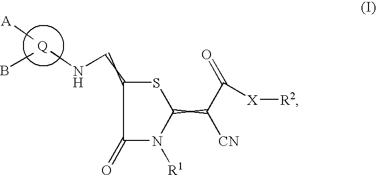
in which
Q stands for aryl or heteroaryl,
A and B, independently of one another, stand for hydrogen, halogen, hydroxy, amino or nitro,
   or
   for C₁-C₃-alkyl or C₁-C₆-alkoxy that is optionally substituted in one or more places, in the same way or differently, with halogen, hydroxy, C₃-C₆-heterocycloalkyl or with the group —NR³R⁴ or —CO(NR³)-M, whereby the heterocycloalkyl itself optionally can be interrupted by one or more nitrogen, oxygen and/or sulfur atoms and/or optionally can be interrupted by one or more —(CO)— or —SO₂— groups in the ring, and/or optionally one or more double bonds can be contained in the ring, and/or the ring itself optionally can be substituted in one or more more places, in the same way or differently, with C₁-C₆-alkyl, C₃-C₆-cycloalkyl, C₁-C₆-hydroxyalkyl or with the group —NR³R⁴, or
   for —NR³(CO)-L, —NR³(CO)—NR³-L, —COR⁶, —CO(NR³)-M, —NR³(CS)NR³R⁴, —NR³SO₂-M, —SO₂—NR³R⁴ or —SO₂(NR³)-M,
L stands for C₁-C₆-alkyl or heteroaryl that is optionally substituted in one or more places, in the same way or differently, with C₁-C₆-hydroxyalkoxy, C₁-C₆-alkoxyalkoxy, C₃-C₆-heterocycloalkyl or with the group —NR³R⁴, whereby the heterocycloalkyl itself optionally can be interrupted by one or more nitrogen, oxygen and/or sulfur atoms, and/or optionally can be interrupted by one or more —(CO)— or —SO₂— groups in the ring, and/or optionally one or more double bonds can be contained in the ring, and/or the ring itself optionally can be substituted in one or more places, in the same way or differently, with C₁-C₆-alkyl, C₃-C₆-cycloalkyl, C₁-C₆-hydroxyalkyl or with the group —NR³R⁴,
M stands for C₁-C₆-alkyl that is optionally substituted in one or more places, in the same way or differently, with the group —NR³R⁴ or C₃-C₆-heterocycloalkyl,
X stands for —NH— or —NR⁵—,
R¹ stands for C₁-C₄-alkyl, C₃-cycloalkyl, allyl or propargyl that is optionally substituted in one or more places, in the same way or differently, with halogen,
R² stands for hydrogen or for C₁-C₆-alkyl, C₁-C₆-alkoxy, C₁-C₆-alkenyl, C₁-C₆-alkinyl, C₃-C₆-cycloalkyl, C₃-C₆-heterocycloalkyl, aryl or heteroaryl that is optionally substituted in one or more places, in the same way or differently, with halogen, hydroxy, cyano, C₁-C₆-alkyl, C₁-C₆-alkoxy, C₁-C₆-hydroxyalkyl, C₃-C₆-cycloalkyl, C₃-C₆-heterocycloalkyl, C₁-C₆-alkinyl, aryl, aryloxy, heteroaryl or with the group —S—C₁-C₆-alkyl, —COR⁶, —NR³R⁴, —NR³(CO)-L or —NR³COOR⁷, whereby the heterocycloalkyl itself optionally can be interrupted by one or more nitrogen, oxygen and/or sulfur atoms, and/or optionally can be interrupted by one or more —(CO)— or —SO₂— groups in the ring, and/or optionally one or more double bonds can be contained in the ring, and whereby aryl, heteroaryl, C₃-C₆-cycloalkyl- and/or the C₃-C₆-heterocycloalkyl ring in each case itself optionally can be substituted in one or more places, in the same way or differently, with cyano, halogen, hydroxy, C₁-C₆-alkyl, C₁-C₆-hydroxyalkyl or C₁-C₆-alkoxy, C₃-C₆-cycloalkyl, C₃-C₆-heterocycloalkyl, aryl, benzyl or heteroaryl that is optionally substituted in one or more places, in the same way or differently, with halogen,
   or
   for the group —NR³R⁴, —NR³(CO)-L, or —NR³(CS)NR³R⁴,
   or
R² and R⁵ together form a C₃-C₆-heterocycloalkyl ring, which is interrupted at least one time by nitrogen and optionally can be interrupted in one or more places by oxygen or sulfur and/or optionally can be interrupted by one or more —(CO)— or —SO₂— groups in the ring, and/or optionally one or more double bonds can be contained in the ring, and/or the ring itself optionally can be substituted in one or more places, in the same way or differently, with cyano, halogen, hydroxy, C₁-C₆-alkyl, C₃-C₆-cycloalkyl, C₁-C₆-hydroxyalkyl, C₁-C₆-alkoxyalkyl or with the group —NR³R⁴ or —COR⁶, and/or can be substituted with aryl or heteroaryl that is optionally substituted in one or more places, in the same way or differently, with halogen, C₁-C₆-alkoxy or with the group —COR⁶,
R³ and R⁴, independently of one another, stand for hydrogen or for C₁-C₆-alkyl, C₁-C₆-alkoxy, —CO—C₁-C₆-alkyl or aryl that is optionally substituted in one or more places, in the same way or differently, with halogen, hydroxy, C₃-C₆-heterocycloalkyl, C₁-C₆-hydroxyalkoxy or with the group —NR³R⁴, whereby the heterocycloalkyl itself optionally can be interrupted by one or more nitrogen, oxygen and/or sulfur atoms, and/or optionally can be interrupted by one or more —(CO)— or —SO₂— groups in the ring, and/or optionally one or more double bonds can be contained in the ring, and whereby the C₃-C₆-heterocycloalkyl ring itself in each case optionally can be substituted in one or more places, in the same way or differently, with cyano, halogen, C₁-C₆-alkyl, C₁-C₆-hydroxyalkyl, C₁-C₆-alkoxy, C₃-C₆-cycloalkyl, or with the group —NR³R⁴ or O—CO—NR³R⁴, or
R³ and R⁴ together form a C₃-C₆-heterocycloalkyl ring, which is interrupted at least once by nitrogen and optionally can be interrupted in one or more places by oxygen or sulfur and/or optionally can be interrupted by one or more —(CO)— or —SO₂— groups in the ring, and/or optionally one or more double bonds can be contained in the ring,
   and/or the heterocycloalkyl ring itself optionally can be substituted in one or more places, in the same way or differently, with C₁-C₆-alkyl, C₃-C₆-cycloalkyl, C₁-C₆-hydroxyalkyl, C₁-C₆-alkoxyalkyl, cyano, hydroxy or with the group —NR³R⁴,
R⁵ stands for C₁-C₆-alkyl, C₁-C₆-alkenyl, or C₁-C₆-alkinyl that is optionally substituted in one or more places, in the same way or differently, with halogen, hydroxy, cyano, C₁-C₆-alkoxy, C₃-C₆-cycloalkyl, C₃-C₆-heterocycloalkyl, or with the group —NR³R⁴, whereby the heterocycloalkyl itself optionally can be interrupted by one or more nitrogen, oxygen and/or sulfur atoms, and/or optionally can be interrupted by one or more —(CO)— or —SO₂— groups in the ring, and/or optionally one or more double bonds can be contained in the ring, and whereby the C₃-C₆-heterocycloalkyl ring itself in each case optionally can be substituted in one or more places, in the same way or differently, with cyano, halogen, C₁-C₆-alkyl, C₁-C₆-hydroxyalkyl, C₁-C₆-alkoxy, C₃-C₆-cycloalkyl, or with the group —NR³R⁴ or —CO—NR³R⁴,
R⁶ stands for hydroxy, C₁-C₆-alkyl, C₁-C₆-alkoxy or the group —NR³R⁴,
R⁷ stands for —(CH₂)ₙ-aryl or —(CH₂)ₙ-heteroaryl and
n stands for 1-6,
   as well as their solvates, hydrates, stereoisomers, diastereomers, enantiomers and salts, with the stipulation that the following compounds do not fall under general formula (I):
{2-Cyano-2-[3-ethyl-4-oxo-5-[1-phenylamino-meth-(E/Z)-ylidene]-thiazolidin-(2-(E or Z))-ylidene]-acetylamino}-acetic acid methyl ester,
2-Cyano-2-[3-ethyl-4-oxo-5-[1-phenylamino-meth-(E/Z)-ylidene]-thiazolidin-(2-(E or Z))-ylidene]-N-pyridin-3-ylmethyl-acetamide,
2-Cyano-2-[3-ethyl-4-oxo-5-[1-phenylamino-meth-(E/Z)-ylidene]-thiazolidin-(2-(E or Z))-ylidene]-N-(3-imidazol-1-yl-propyl)-acetamide,
2-Cyano-2-[3-ethyl-4-oxo-5-[1-phenylamino-meth-(E/Z)-ylidene]-thiazolidin-(2-(E or Z))-ylidene]-N-(4-fluoro-benzyl)-acetamide,
2-Cyano-2-[3-ethyl-4-oxo-5-[1-phenylamino-meth-(E/Z)-ylidene]-thiazolidin-(2-(E or Z))-ylidene]-N-(3-morpholin-4-yl-propyl)-acetamide,
2-Cyano-2-[3-ethyl-4-oxo-5-[1-phenylamino-meth-(E/Z)-ylidene]-thiazolidin-(2-(E or Z))-ylidene]-N-(2-morpholin-4-yl-ethyl)-acetamide,
2-Cyano-2-[3-ethyl-4-oxo-5-[1-phenylamino-meth-(E/Z)-ylidene]-thiazolidin-(2-(E or Z))-ylidene]-N-[3-(2-oxo-pyrrolidin-1-yl)-propyl]-acetamide,
2-Cyano-N-cyclohexyl-2-[3-ethyl-4-oxo-5-[1-phenylamino-meth-(E/Z)-ylidene]-thiazolidin-(2-(E or Z))-ylidene]-acetamide,
4-{2-Cyano-2-[3-ethyl-4-oxo-5-[1-phenylamino-meth-(E/Z)-ylidene]-thiazolidin-(2-(E or Z))-ylidene]-acetylamino}-piperidine-1-carboxylic acid ethyl ester,
2-Cyano-2-[3-ethyl-4-oxo-5-[1-phenylamino-meth-(E/Z)-ylidene]-thiazolidin-(2-(E or Z))-ylidene]-N-(3-hydroxy-propyl)-acetamide,
2-Cyano-2-[3-ethyl-4-oxo-5-[1-phenylamino-meth-(E/Z)-ylidene]-thiazolidin-(2-(E or Z))-ylidene]-N-(4-methoxy-benzyl)-acetamide,
2-Cyano-2-[3-ethyl-4-oxo-5-[1-phenylamino-meth-(E/Z)-ylidene]-thiazolidin-(2-(E or Z))-ylidene]-N-[2-(4-hydroxy-phenyl)-ethyl]-acetamide,
N-Allyl-2-cyano-2-[3-ethyl-4-oxo-5-[1-phenylamino-meth-(E/Z)-ylidene]-thiazolidin-(2-(E or Z))-ylidene]-acetamide,
2-Cyano-2-[3-ethyl-4-oxo-5-[1-phenylamino-meth-(E/Z)-ylidene]-thiazolidin-(2-(E or Z))-ylidene]-N-(2-hydroxy-ethyl)-acetamide,
2-Cyano-2-[3-ethyl-4-oxo-5-[1-phenylamino-meth-(E/Z)-ylidene]-thiazolidin-(2-(E or Z))-ylidene]-N-(4-hydroxy-butyl)-acetamide,
2-Cyano-2-[3-ethyl-4-oxo-5-[1-phenylamino-meth-(E/Z)-ylidene]-thiazolidin-(2-(E or Z))-ylidene]-N-(6-hydroxy-hexyl)-acetamide,
2-Cyano-2-[3-ethyl-4-oxo-5-[1-phenylamino-meth-(E/Z)-ylidene]-thiazolidin-(2-(E or Z))-ylidene]-acetamide,
2-Cyano-N-ethyl-2-[3-ethyl-4-oxo-5-[1-phenylamino-meth-(E/Z)-ylidene]-thiazolidin-(2-(E or Z))-ylidene]-acetamide,
2-Cyano-2-[3-ethyl-5-[1-(4-methoxy-phenylamino)-meth-(E/Z)-ylidene]-4-oxo-thiazolidin-(2-(E or Z))-ylidene]-N,N-dimethyl-acetamide,
2-Cyano-2-[3-ethyl-4-oxo-5-[1-phenylamino-meth-(E/Z)-ylidene]-thiazolidin-(2-(E or Z))-ylidene]-N,N-dimethyl-acetamide,
6-{[2-[1-Cyano-1-dimethylcarbamoyl-meth-(E or Z)-ylidene]-3-ethyl-4-oxo-thiazolidin-(5-(E/Z))-ylidenemethyl]-amino}-naphthalene-2-carboxylic acid,
4-{[2-[1-Cyano-1-dimethylcarbamoyl-meth-(E or Z)-ylidene]-3-ethyl-4-oxo-thiazolidin-(5-(E/Z))-ylidenemethyl]-amino}-benzoic acid,
2-Cyano-2-[3-ethyl-5-[1-(4-hydroxy-phenylamino)-meth-(E/Z)-ylidene]-4-oxo-thiazolidin-(2-(E or Z))-ylidene]-N,N-dimethyl-acetamide,
4-{[2-[1-Cyano-1-dimethylcarbamoyl-meth-(E or Z)-ylidene]-3-ethyl-4-oxo-thiazolidin-(5-(E/Z))-ylidenemethyl]-amino}-benzamide,
2-Cyano-2-[3-ethyl-5-[1-(4-hydroxymethyl-phenylamino)-meth-(E/Z)-ylidene]-4-oxo-thiazolidin-(2-(E or Z))-ylidene]-N,N-dimethyl-acetamide,
   are suitable inhibitors of the kinases of the polo family.

The compounds of general formula I according to the invention essentially inhibit the polo-like kinases, upon which is based their action against, for example, cancer, such as solid tumors and leukemia; auto-immune diseases, such as psoriasis, alopecia, and multiple sclerosis, chemotherapy agent-induced alopecia and mucositis; cardiovascular diseases, such as stenoses, arterioscleroses and restenoses; infectious diseases, such as, e.g., by unicellular parasites, such as trypanosoma, toxoplasma or plasmodium, or produced by fungi; nephrological diseases, such as, e.g., glomerulonephritis, chronic neurodegenerative diseases, such as Huntington's disease, amyotropic lateral sclerosis, Parkinson's disease, AIDS dementia and Alzheimer's disease; acute neurodegenerative diseases, such as ischemias of the brain and neurotraumas; viral infections, such as, e.g., cytomegalic infections, herpes, hepatitis B and C, and HIV diseases.

Stereoisomers can be defined as E/Z- and R/S-isomers as well as mixtures that consist of E/Z- and R/S-isomers.

Alkyl is defined in each case as a straight-chain or branched alkyl radical, such as, for example, methyl, ethyl, propyl, isopropyl, butyl, isobutyl, sec.-butyl, tert.-butyl, pentyl, isopentyl, hexyl, heptyl, octyl, nonyl and decyl.

Alkoxy is defined in each case as a straight-chain or branched alkoxy radical, such as, for example, methyloxy, ethyloxy, propyloxy, isopropyloxy, butyloxy, isobutyloxy, sec.-butyloxy, pentyloxy, isopentyloxy, hexyloxy, heptyloxy, octyloxy, nonyloxy or decyloxy.

The alkenyl substituents in each case are straight-chain or branched, and, for example, the following radicals are meant: vinyl, propen-1-yl, propen-2-yl, but-1-en-1-yl, but-1-en-2-yl, but-2-en-1-yl, but-2-en-2-yl, 2-methyl-prop-2-en-1-yl, 2-methyl-prop-1-en-1-yl, but-1-en-3-yl, but-3-en-1-yl, and allyl.

Alkinyl is defined in each case as a straight-chain or branched alkinyl radical that contains 2-6, preferably 2-4 C atoms. For example, the following radicals can be mentioned: acetylene, propin-1-yl, propin-3-yl, but-1-in-1-yl, but-1-in-4-yl, but-2-in-1-yl, but-1-in-3-yl, etc.

Heterocycoalkyl stands for an alkyl ring that comprises 3-6 carbon atoms, which instead of carbon contains one or more heteroatoms, the same or different, such as, e.g., oxygen, sulfur or nitrogen, and/or optionally can be interrupted by one or more —(CO)— or —SO₂— groups in the ring, and/or optionally one or more double bonds can be contained in the ring, and can contain another substituent on one or more carbon, nitrogen or sulfur atoms, optionally independently of one another. Substituents on the heterocycloalkyl ring can be: cyano, halogen, hydroxy, C₁-C₆-alkyl, C₁-C₆-alkoxy, C₁-C₆-alkoxyalkyl, C₁-C₆-hydroxyalkyl, C₃-C₆-cycloalkyl, aryl or the group —NR³R⁴, —CO—NR³R⁴, —SO₂R³ or —SO₂NR³R⁴.

As heterocycloalkyls, there can be mentioned, e.g.: oxiranyl, oxethanyl, aziridinyl, azetidinyl, tetrahydrofuranyl, pyrrolidinyl, dioxolanyl, imidazolidinyl, pyrazolidinyl, dioxanyl, piperidinyl, morpholinyl, dithianyl, thiomorpholinyl, piperazinyl, trithianyl, quinuclidinyl, pyrrolidonyl, N-methylpyrrolidinyl, 2-hydroxymethylpyrrolidinyl, 3-hydroxypyrrolidinyl, N-methylpiperazinyl, N-acetylpiperazinyl, N-methylsulfonylpiperazinyl, 4-hydroxypiperidinyl, 4-aminocarbonylpiperidinyl, 2-hydroxyethylpiperidinyl, 4-hydroxymethylpiperidinyl, nortropinyl, 1,1-dioxo-thiomorpholinyl, etc.

Cycloalkyls are defined as monocyclic alkyl rings, such as cyclopropyl, cyclobutyl, cyclopentyl, cyclohexyl or cycloheptyl, but also bicyclic rings or tricyclic rings, such as, for example, adamantanyl. The cycloalkyl can optionally also be benzocondensed, such as, e.g. (tetralin)yl, etc.

Halogen is defined in each case as fluorine, chlorine, bromine or iodine.

The aryl radical in each case has 6-12 carbon atoms, such as, for example, naphthyl, biphenyl and in particular phenyl.

In each case, the heteroaryl radical comprises 3-16 ring atoms and, instead of carbon, can contain one or more heteroatoms, the same or different, such as oxygen, nitrogen or sulfur in the ring, and can be mono-, bi- or tricyclic, and can in addition in each case be benzocondensed.

For example, there can be mentioned:

Thienyl, furanyl, pyrrolidinyl, oxazolyl, thiazolyl, imidazolyl, pyrazolyl, isoxazolyl, isothiazolyl, oxadiazolyl, triazolyl, thiadiazolyl, etc., and benzo derivatives thereof, such as, e.g., benzofuranyl, benzothienyl, benzoxazolyl, benzimidazolyl, indazolyl, indolyl, isoindolyl, etc.; or pyridyl, pyridazinyl, pyrimidinyl, pyrazinyl, triazinyl, etc., and benzo derivatives thereof, such as, e.g., quinolyl, isoquinolyl, etc.; or oxepinyl, azocinyl, indolizinyl, indolyl, indolinyl, isoindolyl, indazolyl, benzimidazolyl, purinyl, etc., and benzo derivatives thereof; or quinolinyl, isoquinolinyl, cinnolinyl, phthalazinyl, quinazolinyl, quinoxalinyl, naphthyridinyl, pteridinyl, carbazolyl, acridinyl, phenazinyl, phenothiazinyl, phenoxazinyl, xanthenyl, tetralinyl, etc.

Preferred heteroaryl radicals, are, for example, 5-ring heteroaromatic compounds, such as thiophene, furanyl, oxazolyl, thiazole, imidazolyl and benzo derivatives thereof, and 6-ring-heteroaromatic compounds, such as pyridinyl, pyrimidinyl, triazinyl, quinolinyl, isoquinolinyl and benzo derivatives thereof.

The aryl radical comprises 3-12 carbon atoms in each case and can be benzocondensed in each case.

For example, there can be mentioned: cyclopropenyl, cyclopentadienyl, phenyl, tropyl, cyclooctadienyl, indenyl, naphthyl, azulenyl, biphenyl, fluorenyl, anthracenyl, tetralinyl, etc.

Isomers are defined as chemical compounds of the same summation formula but different chemical structure. In general, constitutional isomers and stereoisomers are distinguished.

Constitutional isomers have the same summation formula but are distinguished by the way in which their atoms or groups of atoms are linked. These include functional isomers, positional isomers, tautomers or valence isomers.

In principle, stereoisomers have the same structure (constitution)- and thus also the same summation formula—but are distinguished by the spatial arrangement of the atoms.

In general, configurational isomers and conformational isomers are distinguished. Configurational isomers are stereoisomers that can be converted into one another only by bond breaking. These include enantiomers, diastereomers and E/Z (cis/trans) isomers.

Enantiomers are stereoisomers that behave toward one another like image and mirror image and do not have any symmetry plane. All stereoisomers that are not enantiomers are referred to as diastereomers. E/Z (cis/trans) isomers of double bonds are a special case.

Conformational isomers are stereoisomers that can be converted into one another by the turning of single bonds.

To differentiate the types of isomerism from one another, see also the IUPAC rules, Section E (Pure Appl. Chem. 45, 11-30, 1976).

The compounds of general formula I according to the invention also contain the possible tautomeric forms and comprise the E or Z isomers or, if a chiral center is present, also the racemates and enantiomers. Among the latter, double-bond isomers are also included.

The compounds according to the invention can also be present in the form of solvates, in particular hydrates, whereby the compounds according to the invention consequently contain polar solvents, in particular water, as structural elements of the crystal lattice of the compounds according to the invention. The proportion of polar solvent, in particular water, can be present in a stoichiometric or even an unstoichiometric ratio. In the case of stoichiometric solvates and hydrates, hemi-, (semi-), mono-, sesqui-, di-, tri-, tetra-, penta, etc., solvates or hydrates are also indicated.

If an acid group is included, the physiologically compatible salts of organic and inorganic bases are suitable as salts, such as, for example, the readily soluble alkali and alkaline-earth salts, as well as N-methyl-glucamine, dimethyl-glucamine, ethyl-glucamine, lysine, 1,6-hexadiamine, ethanolamine, glucosamine, sarcosine, serinol, tris-hydroxy-methyl-amino-methane, aminopropane diol, Sovak base, and 1-amino-2,3,4-butanetriol.

If a basic group is included, the physiologically compatible salts of organic and inorganic acids are suitable, such as hydrochloric acid, sulfuric acid, phosphoric acid, citric acid, tartaric acid, i.a.

Preferred in particular are those compounds of general formula I, in which
Q stands for phenyl, naphthyl, quinolinyl, benzimidazolyl, indolyl, indazolyl, thiazolyl, imidazolyl or pyridyl,
A and B, independently of one another, stand for hydrogen, halogen, hydroxy, amino or nitro
   or
   for C₁-C₃-alkyl or C₁-C₆-alkoxy that is optionally substituted in one or more places, in the same way or differently, with halogen, hydroxy, C₃-C₆-heterocycloalkyl or with the group —NR³R⁴ or —CO(NR³)-M, whereby the heterocycloalkyl itself optionally can be interrupted by one or more nitrogen, oxygen and/or sulfur atoms, and/or optionally can be interrupted by one or more —(CO)— or —SO₂— groups in the ring, and/or optionally one or more double bonds can be contained in the ring, and/or the ring itself optionally can be substituted in one or more places, in the same way or differently, with C₁-C₆-alkyl, C₃-C₆-cycloalkyl, C₁-C₆-hydroxyalkyl or with the group —NR³R⁴,
   or
   for —NR³(CO)-L, —NR³(CO)—NR³-L, —COR⁶, —CO(NR³)-M, —NR³(CS)NR³R⁴, —NR³SO₂-M, —SO₂—NR³R⁴ or —SO₂(NR³)-M,
L stands for C₁-C₆-alkyl or heteroaryl that is optionally substituted in one or more places, in the same way or differently, with C₁-C₆-hydroxyalkoxy, C₁-C₆-alkoxyalkoxy, C₃-C₆-heterocycloalkyl or with the group —NR³R⁴, whereby the heterocycloalkyl itself optionally can be interrupted by one or more nitrogen, oxygen and/or sulfur atoms, and/or optionally can be interrupted by one or more —(CO)— or —SO₂— groups in the ring, and/or optionally one or more double bonds can be contained in the ring, and/or the ring itself optionally can be substituted in one or more places, in the same way or differently, with C₁-C₆-alkyl, C₃-C₆-cycloalkyl, C₁-C₆-hydroxyalkyl or with the group —NR³R⁴,
M stands for C₁-C₆-alkyl that is optionally substituted in one or more places, in the same way or differently, with the group —NR³R⁴ or C₃-C₆-heterocycloalkyl,
X stands for —NH— or —NR⁵—,
R¹ stands for C₁-C₄-alkyl, C₃-cycloalkyl, allyl or propargyl that is optionally substituted in one or more places, in the same way or differently, with halogen,
R² stands for hydrogen or for C₁-C₆-alkyl, C₁-C₆-alkoxy, C₁-C₆-alkenyl, C₁-C₆-alkinyl, C₃-C₆-cycloalkyl, C₃-C₆-heterocycloalkyl, aryl or heteroaryl that is optionally substituted in one or more places, in the same way or differently, with halogen, hydroxy, cyano, C₁-C₆-alkyl, C₁-C₆-alkoxy, C₁-C₆-hydroxyalkyl, C₃-C₆-cycloalkyl, C₃-C₆-heterocycloalkyl, C₁-C₆-alkinyl, aryl, aryloxy, heteroaryl or with the group —S—C₁-C₆-alkyl, —COR⁶, —NR³R⁴, —NR³(CO)-L or —NR³COOR⁷, whereby the heterocycloalkyl itself optionally can be interrupted by one or more nitrogen, oxygen and/or sulfur atoms, and/or optionally can be interrupted by one or more —(CO)— or —SO₂— groups in the ring, and/or optionally one or more double bonds can be contained in the ring, and whereby aryl, heteroaryl, C₃-C₆-cycloalkyl- and/or the C₃-C₆-heterocycloalkyl ring in each case itself optionally can be substituted in one or more places, in the same way or differently, with cyano, halogen, hydroxy, C₁-C₆-alkyl, C₁-C₆-hydroxyalkyl, or C₁-C₆-alkoxy, C₃-C₆-cycloalkyl, C₃-C₆-heterocycloalkyl, aryl, benzyl or heteroaryl that is optionally substituted in one or more places, in the same way or differently, with halogen,
   or
   for the group —NR³R⁴, —NR³(CO)-L, or —NR³(CS)NR³R⁴,
   or
R² and R⁵ together form a C₃-C₆-heterocycloalkyl ring, which is interrupted at least once by nitrogen and optionally can be interrupted in one or more places by oxygen or sulfur and/or optionally can be interrupted by one or more —(CO)— or —SO₂— groups in the ring, and/or optionally one or more double bonds can be contained in the ring, and/or the ring itself optionally can be substituted in one or more places, in the same way or differently, with cyano, halogen, hydroxy, C₁-C₆-alkyl, C₃-C₆-cycloalkyl, C₁-C₆-hydroxyalkyl, C₁-C₆-alkoxyalkyl or with the group —NR³R⁴ or —COR⁶, and/or with aryl or heteroaryl that is optionally substituted in one or more places, in the same way or differently, with halogen, C₁-C₆-alkoxy or with the group —COR⁶,
R³ and R⁴, independently of one another, stand for hydrogen or for C₁-C₆-alkyl, C₁-C₆-alkoxy, —CO—C₁-C₆-alkyl or aryl that is optionally substituted in one or more places, in the same way or differently, with halogen, hydroxy, C₃-C₆-heterocycloalkyl, C₁-C₆-hydroxyalkoxy or with the group —NR³R⁴, whereby the heterocycloalkyl itself optionally can be interrupted by one or more nitrogen, oxygen and/or sulfur atoms, and/or optionally can be interrupted by one or more —(CO)— or —SO₂— groups in the ring, and/or optionally one or more double bonds can be contained in the ring, and whereby the C₃-C₆-heterocycloalkyl ring itself in each case optionally can be substituted in one or more places, in the same way or differently, with cyano, halogen, C₁-C₆-alkyl, C₁-C₆-hydroxyalkyl, C₁-C₆-alkoxy, C₃-C₆-cycloalkyl, or with the group —NR³R⁴ or —CO—NR³R⁴,
   or
R³ and R⁴ together form a C₃-C₆-heterocycloalkyl ring, which is interrupted by nitrogen at least once and optionally can be interrupted in one or more places by oxygen or sulfur, and/or optionally can be interrupted by one or more —(CO)— or —SO₂— groups in the ring, and/or optionally one or more double bonds can be contained in the ring, and/or the heterocycloalkyl ring itself optionally can be substituted in one or more places, in the same way or differently, with C₁-C₆-alkyl, C₃-C₆-cycloalkyl, C₁-C₆-hydroxyalkyl, C₁-C₆-alkoxyalkyl, cyano, hydroxy or with the group —NR³R⁴,
R⁵ stands for C₁-C₆-alkyl, C₁-C₆-alkenyl, or C₁-C₆-alkinyl that is optionally substituted in one or more places, in the same way or differently, with halogen, hydroxy, cyano, C₁-C₆-alkoxy, C₃-C₆-cycloalkyl, C₃-C₆-heterocycloalkyl, or with the group —NR³R⁴, whereby the heterocycloalkyl itself optionally can be interrupted by one or more nitrogen, oxygen and/or sulfur atoms and/or optionally can be interrupted by one or more —(CO)— or —SO₂— groups in the ring, and/or optionally one or more double bonds can be contained in the ring, and whereby the C₃-C₆-heterocycloalkyl ring itself in each case optionally can be substituted in one or more places, in the same way or differently, with cyano, halogen, C₁-C₆-alkyl, C₁-C₆-hydroxyalkyl, C₁-C₆-alkoxy, C₃-C₆-cycloalkyl, or with the group —NR³R⁴ or —CO—NR³R⁴,
R⁶ stands for hydroxy, C₁-C₆-alkyl, C₁-C₆-alkoxy or the group —NR³R⁴,
R⁷ stands for —(CH₂)ₙ-aryl or —(CH₂)ₙ-heteroaryl and
n stands for 1-6,
   as well as their solvates, hydrates, stereoisomers, diastereomers, enantiomers and salts.

Especially preferred are those compounds of general formula I, in which
Q stands for phenyl, naphthyl or indolyl,
A and B, independently of one another, stand for hydrogen, halogen, hydroxy, amino or nitro
   or
   for C₁-C₃-alkyl or C₁-C₆-alkoxy that is optionally substituted in one or more places, in the same way or differently, with halogen, hydroxy, C₃-C₆-heterocycloalkyl or with the group —NR³R⁴ or —CO(NR³)-M, whereby the heterocycloalkyl itself optionally can be interrupted by one or more nitrogen, oxygen and/or sulfur atoms, and/or optionally can be interrupted by one or more —(CO)— or —SO₂— groups in the ring, and/or optionally one or more double bonds can be contained in the ring, and/or the ring itself optionally can be substituted in one or more places, in the same way or differently, with C₁-C₆-alkyl, C₃-C₆-cycloalkyl, C₁-C₆-hydroxyalkyl or with the group —NR³R⁴,
   or
   for —NR³(CO)-L, —NR³(CO)—NR³-L, —COR⁶, —CO(NR³)-M, —NR³(CS)NR³R⁴, —NR³SO₂-M, —SO₂—NR³R⁴ or —SO₂(NR³)-M,
L stands for C₁-C₆-alkyl or heteroaryl that is optionally substituted in one or more places, in the same way or differently, with C₁-C₆-hydroxyalkoxy, C₁-C₆-alkoxyalkoxy, C₃-C₆-heterocycloalkyl or with the group —NR³R⁴, whereby the heterocycloalkyl itself optionally can be interrupted by one or more nitrogen, oxygen and/or sulfur atoms, and/or optionally can be interrupted by one or more —(CO)— or —SO₂— groups in the ring, and/or optionally one or more double bonds can be contained in the ring, and/or the ring itself optionally can be substituted in one or more places, in the same way or differently, with C₁-C₆-alkyl, C₃-C₆-cycloalkyl, C₁-C₆-hydroxyalkyl or with the group —NR³R⁴,
M stands for C₁-C₆-alkyl that is optionally substituted in one or more places, in the same way or differently, with the group —NR³R⁴ or C₃-C₆-heterocycloalkyl,
X stands for —NH— or —NR⁵—,
R¹ stands for C₁-C₄-alkyl, C₃-cycloalkyl, allyl or propargyl that is optionally substituted in one or more places, in the same way or differently, with halogen,
R² stands for hydrogen or for C₁-C₆-alkyl, C₁-C₆-alkoxy, C₁-C₆-alkenyl, C₁-C₆-alkinyl, C₃-C₆-cycloalkyl, C₃-C₆-heterocycloalkyl, aryl or heteroaryl that is optionally substituted in one or more places, in the same way or differently, with halogen, hydroxy, cyano, C₁-C₆-alkyl, C₁-C₆-alkoxy, C₁-C₆-hydroxyalkyl, C₃-C₆-cycloalkyl, C₃-C₆-heterocycloalkyl, C₁-C₆-alkinyl, aryl, aryloxy, heteroaryl or with the group —S—C₁-C₆-alkyl, —COR⁶, —NR³R⁴, —NR³(CO)-L or —NR³COOR⁷,
   whereby the heterocycloalkyl itself optionally can be interrupted by one or more nitrogen, oxygen and/or sulfur atoms and/or optionally can be interrupted by one or more —(CO)— or —SO₂— groups in the ring, and/or optionally one or more double bonds can be contained in the ring,
   and whereby aryl, heteroaryl, C₃-C₆-cycloalkyl- and/or the C₃-C₆-heterocycloalkyl ring in each case itself optionally can be substituted in one or more places, in the same way or differently, with cyano, halogen, hydroxy, C₁-C₆-alkyl, C₁-C₆-hydroxyalkyl, or C₁-C₆-alkoxy, C₃-C₆-cycloalkyl, C₃-C₆-heterocycloalkyl, aryl, benzyl or heteroaryl that is optionally substituted in one or more places, in the same way or differently, with halogen,
   or
   for the group —NR³R⁴, —NR³(CO)-L, or —NR³(CS)NR³R⁴,
   or
R² and R⁵ together form a C₃-C₆-heterocycloalkyl ring, which is interrupted at least once by nitrogen and optionally can be interrupted in one or more places by oxygen or sulfur and/or optionally can be interrupted by one or more —(CO)— or —SO₂— groups in the ring, and/or optionally one or more double bonds can be contained in the ring, and/or the ring itself optionally can be substituted in one or more places, in the same way or differently, with cyano, halogen, hydroxy, C₁-C₆-alkyl, C₃-C₆-cycloalkyl, C₁-C₆-hydroxyalkyl, C₁-C₆-alkoxyalkyl or with the group —NR³R⁴ or —COR⁶, and/or can be substituted with aryl or heteroaryl that is optionally substituted in one or more places, in the same way or differently, with halogen, C₁-C₆-alkoxy or with the group —COR⁶,
R³ and R⁴, independently of one another, stand for hydrogen or for C₁-C₆-alkyl, C₁-C₆-alkoxy, —CO—C₁-C₆-alkyl or aryl that is optionally substituted in one or more places, in the same way or differently, with halogen, hydroxy, C₃-C₆-heterocycloalkyl, C₁-C₆-hydroxyalkoxy or with the group —NR³R⁴, whereby the heterocycloalkyl itself optionally can be interrupted by one or more nitrogen, oxygen and/or sulfur atoms, and/or optionally can be interrupted by one or more —(CO)— or —SO₂— groups in the ring, and/or optionally one or more double bonds can be contained in the ring, and whereby the C₃-C₆-heterocycloalkyl ring itself in each case optionally can be substituted in one or more places, in the same way or differently, with cyano, halogen, C₁-C₆-alkyl, C₁-C₆-hydroxyalkyl, C₁-C₆-alkoxy, C₃-C₆-cycloalkyl, or with the group —NR³R⁴ or —CO—NR³R⁴,
   or
R³ and R⁴ together form a C₃-C₆-heterocycloalkyl ring, which is interrupted at least once by nitrogen, and optionally can be interrupted in one or more places by oxygen or sulfur, and/or optionally can be interrupted by one or more —(CO)— or —SO₂— groups in the ring, and/or optionally one or more double bonds can be contained in the ring,
   and/or the heterocycloalkyl ring itself optionally can be substituted in one or more places, in the same way or differently, with C₁-C₆-alkyl, C₃-C₆-cycloalkyl, C₁-C₆-hydroxyalkyl, C₁-C₆-alkoxyalkyl, cyano, hydroxy or with the group —NR³R⁴,
R⁵ stands for C₁-C₆-alkyl, C₁-C₆-alkenyl, or C₁-C₆-alkinyl that is optionally substituted in one or more places, in the same way or differently, with halogen, hydroxy, cyano, C₁-C₆-alkoxy, C₃-C₆-cycloalkyl, C₃-C₆-heterocycloalkyl, or with the group —NR³R⁴, whereby the heterocycloalkyl itself optionally can be interrupted by one or more nitrogen, oxygen and/or sulfur atoms, and/or optionally can be interrupted by one or more —(CO)— or —SO₂— groups in the ring, and/or optionally one or more double bonds can be contained in the ring, and whereby the C₃-C₆-heterocycloalkyl ring itself in each case optionally can be substituted in one or more places, in the same way or differently, with cyano, halogen, C₁-C₆-alkyl, C₁-C₆-hydroxyalkyl, C₁-C₆-alkoxy, C₃-C₆-cycloalkyl, or with the group —NR³R⁴ or —CO—NR³R⁴,
R⁶ stands for hydroxy, C₁-C₆-alkyl, C₁-C₆-alkoxy or the group —NR³R⁴,
R⁷ stands for —(CH₂)ₙ-aryl or —(CH₂)ₙ-heteroaryl and
n stands for 1-6,
   as well as their solvates, hydrates, stereoisomers, diastereomers, enantiomers and salts.

In particular, those compounds of general formula (I) are preferred in which
Q stands for phenyl, naphthyl or indolyl,
A and B, independently of one another, stand for hydrogen, halogen, hydroxy, amino or nitro
   or
   for C₁-C₃-alkyl or C₁-C₆-alkoxy that is optionally substituted in one or more places, in the same way or differently, with pyrrolidinyl, piperidinyl, piperazinyl or with the group —N(C₁-C₆-alkyl)₂, whereby pyrrolidinyl, piperidinyl or piperazinyl itself optionally can be substituted in one or more places, in the same way or differently, with C₁-C₆-alkyl or C₁-C₆-hydroxyalkyl,
   or
   for —CO(NH)-M, —CO(NCH₃)-M, —NH(CO)-L, —NH(CO)—NH-L, —SO₂(NH)-M or —SO₂(NCH₃)-M,
L stands for C₁-C₆-alkyl or pyridyl that is optionally substituted in one or more places, in the same way or differently, with C₁-C₆-hydroxyalkoxy, C₁-C₆-alkoxyalkoxy, pyrrolidinyl, piperazinyl or with the group —N(C₁-C₆-alkyl)₂, whereby the pyrrolidinyl or piperazinyl itself optionally can be substituted in one or more places, in the same way or differently, with C₁-C₆-alkyl,
M stands for C₁-C₆-alkyl that is optionally substituted in one or more places, in the same way or differently, with the group —N(C₁-C₆-alkyl)₂ or pyrrolidinyl,
X stands for —NH— or —NR⁵—,
R¹ stands for C₁-C₄-alkyl that is optionally substituted in one or more places, in the same way or differently, with halogen,
R² stands for hydrogen or for C₁-C₆-alkyl, C₁-C₆-alkenyl, C₁-C₆-alkinyl, C₃-C₆-cycloalkyl, pyrrolidinyl, piperidinyl, phenyl, tetralinyl or indolyl that is optionally substituted in one or more places, in the same way or differently, with halogen, hydroxy, cyano, C₁-C₆-alkyl, C₁-C₆-alkoxy, C₁-C₆-hydroxyalkyl, C₃-C₆-cycloalkyl, tetrahydrofuranyl, pyrrolidinyl, piperazinyl, morpholinyl, phenyl, phenoxy, biphenyl, naphthyl, thienyl, furanyl, tetrazolyl, pyridyl or with the group —S—C₁-C₆-alkyl, —CONH₂, —COO—C₁-C₆-alkyl, —N(C₁-C₆-alkyl)₂, —N(C₁-C₆-alkyl)phenyl, or —NH(CO)-L,
   whereby phenyl, furanyl, C₃-C₆-cycloalkyl, piperidinyl or piperazinyl in each case itself optionally can be substituted in one or more places, in the same way or differently, with C₁-C₆-alkyl, C₁-C₆-alkoxy, cyano, halogen, hydroxy, phenyl, benzyl, or morpholinyl, and the C₁-C₆-alkyl or C₁-C₆-alkoxy itself optionally can be substituted in one or more places, in the same way or differently, with halogen,
   or
   for the group —N(C₁-C₆-alkyl)₂, —NH(CO)-L, or —NCH₃(CS)NHCH₃,
   or
R² and R⁵ together form aziridinyl, azetidinyl, morpholinyl, pyrrolidinyl, piperidinyl or piperazinyl, whereby aziridinyl, azetidinyl, morpholinyl, pyrrolidinyl, piperidinyl or piperazinyl itself optionally can be substituted in one or more places, in the same way or differently, with hydroxy, C₁-C₆-alkyl, C₁-C₆-hydroxyalkyl, C₁-C₆-alkoxyalkyl or with the group —CONH₂, —CO—C₁-C₆-alkyl or —COO—C₁-C₆-alkyl, and/or can be substituted with phenyl, benzyl or pyridyl that is optionally substituted in one or more places, in the same way or differently, with halogen or C₁-C₆-alkoxy, and
R⁵ stands for C₁-C₆-alkyl or C₁-C₆-alkenyl that is optionally substituted in one or more places, in the same way or differently, with C₁-C₆-alkoxy,
   as well as their solvates, hydrates, stereoisomers, diastereomers, enantiomers and salts.

Primarily those compounds of general formula (I) are preferred, in which
Q stands for phenyl, naphthyl or indolyl,
A and B, independently of one another, stand for hydrogen, halogen, hydroxy, amino or nitro,
   or
   for C₁-C₃-alkyl or C₁-C₃-alkoxy that is optionally substituted in one or more places, in the same way or differently, with pyrrolidinyl, piperidinyl, piperazinyl or with the group —N(CH₃)₂, whereby pyrrolidinyl, piperidinyl or piperazinyl itself optionally can be substituted in one or more places, in the same way or differently, with C₁-C₃-alkyl or C₁-C₃-hydroxyalkyl,
   or
   for the group —CO—NH—(CH₂)₂—N(CH₃)₂, —CO—NH—(CH₂)₂—N(C₂H₅)₂, —CO—N(CH₃)—(CH₂)₂—N(CH₃)₂, 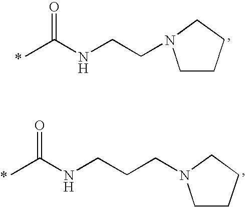
   —NH(CO)—C(CH₃)₃, —NH(CO)—(CH₂)—O(CH₂)₂—OCH₃, —NH(CO)—(CH₂)₂—N(C₂H₅)₂, 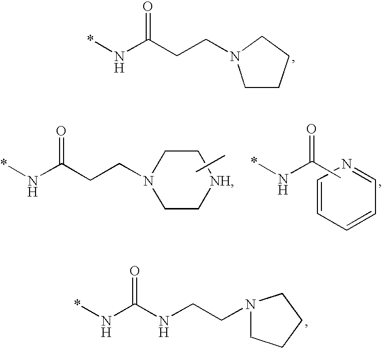
   or —SO₂—NH—(CH₂)₂—N(CH₃)₂ or —SO₂—N(CH₃)—(CH₂)₂—N(CH₃)₂,
X stands for —NH— or —NR⁵—,
R¹ stands for C₁-C₃-alkyl that is optionally substituted in one or more places, in the same way or differently, with halogen,
R² stands for hydrogen or for C₁-C₆-alkyl, C₁-C₄-alkenyl, C₁-C₄-alkinyl, C₃-C₆-cycloalkyl, piperidinyl, phenyl, pyrrolidinyl, indolyl or tetralinyl that is optionally substituted in one or more places, in the same way or differently with halogen, hydroxy, cyano, C₁-C₆-alkyl, C₁-C₆-hydroxyalkyl, methoxy, C₃-C₆-cycloalkyl, tetrahydrofuranyl, pyrrolidinyl, piperazinyl, morpholinyl, phenyl, phenoxy, biphenyl, naphthyl, thienyl, furanyl, tetrazolyl or pyridyl or with the group —S—CH₃, —COOCH₃, —COOC₂H₅, —CO—NH₂, —OCF₃, —N(CH₃)— phenyl, —N(C₁-C₄-alkyl)₂, or —NH(CO)—CH₃, whereby phenyl, furanyl, C₃-C₆-cycloalkyl, piperidinyl or piperazinyl optionally in each case itself can be substituted in one or more places, in the same way or differently, with cyano, halogen, hydroxy, C₁-C₃-alkyl, C₁-C₃-hydroxyalkyl, methoxy, morpholinyl, phenyl or benzyl,
   or
   for the group —N(CH₃)₂, —N(CH₃)(CS)NHCH₃, —NH(CO)—CH₃, —NH(CO)-pyridyl, or —NH(CO)-pyridinyl,
   or
R² and R together form one of the following rings: 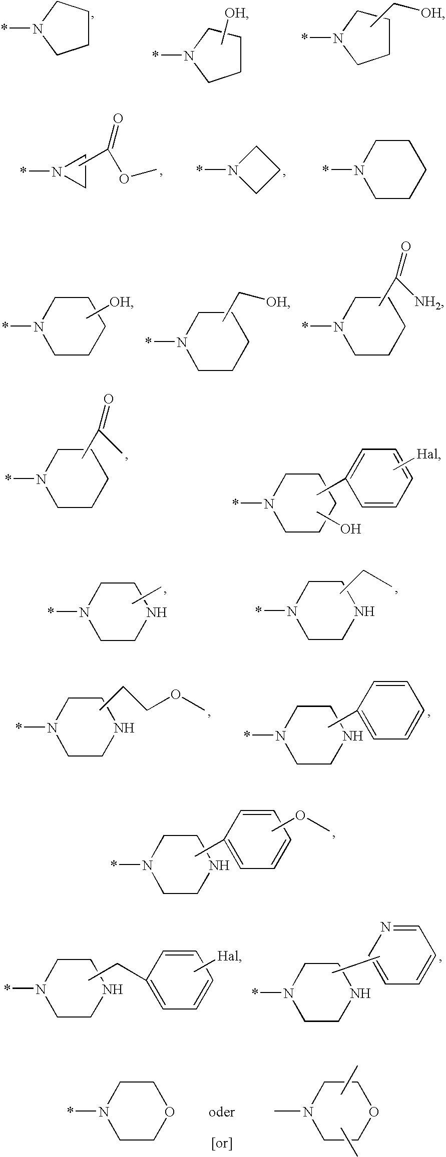
and
R⁵ stands for C₁-C₃-alkyl or C₁-C₃-alkenyl that is optionally substituted in one or more places, in the same way or differently, with C₁-C₆-alkoxy,
   as well as their solvates, hydrates, stereoisomers, diastereomers, enantiomers and salts.

The position that is identified by * in the formulas indicates the point of linkage to the remainder of the formula.

Also subjects of the invention are compounds of general formula I, in which
Q stands for phenyl, naphthyl, quinolinyl, benzimidazolyl, indolyl, indazolyl, thiazolyl, imidazolyl or pyridyl,
A and B, independently of one another, stand for hydrogen, halogen, hydroxy, amino or nitro,
   or
   for C₁-C₃-alkyl or C₁-C₆-alkoxy that is optionally substituted in one or more places, in the same way or differently, with hydroxy, C₃-C₆-heterocycloalkyl or with the group —NR³R⁴ or —CO(NR³)(CH₂)ₙNR³R⁴, whereby the heterocycloalkyl itself optionally can be interrupted by one or more nitrogen, oxygen and/or sulfur atoms, and/or optionally can be interrupted by one or more —(CO)— or —SO₂— groups in the ring, and/or optionally one or more double bonds can be contained in the ring, and/or the ring itself optionally can be substituted in one or more places, in the same way or differently, with C₁-C₆-alkyl, C₃-C₆-cycloalkyl, C₁-C₆-hydroxyalkyl or with the group —NR³R⁴,
   or
   for COR⁶, —CO(NR³)(CH₂)ₙNR³R⁴, —NR³(CO)—C₁-C₆-alkyl,
   —NR³(CO)(CH₂), C₁-C₆-alkoxy, —NR³(CO)(CH₂)ₙC₁-C₆-alkoxyalkoxy, —NR³(CO)(CH₂)ₙNR³R⁴, —NR³(CO)NR³R⁴, —NR³(CS)NR³R⁴, —NR³ SO₂—C₁-C₆-alkyl, —NR³SO₂—(CH₂)ₙNR³R⁴, —SO₂—NR³R⁴ or —SO₂(NR³)(CH₂)ₙNR³R⁴,
X stands for oxygen, —NH— or —NR⁵—,
R¹ stands for C₁-C₃-alkyl, C₃-cycloalkyl, allyl or propargyl that is optionally substituted in one or more places, in the same way or differently, with halogen,
R² stands for hydrogen, or for C₁-C₆-alkyl, C₁-C₆-alkoxy, C₁-C₆-alkenyl, C₁-C₆-alkinyl, C₃-C₆-cycloalkyl, C₃-C₆-heterocycloalkyl, aryl or heteroaryl that is optionally substituted in one or more places, in the same way or differently, with halogen, hydroxy, cyano, C₁-C₆-alkyl, C₁-C₆-alkoxy, C₁-C₆-hydroxyalkyl, C₃-C₆-cycloalkyl, C₃-C₆-heterocycloalkyl, aryl, heteroaryl or with the group —S—C₁-C₆-alkyl, —COR⁶, —NR³R⁴, —NR³(CO)—C₁-C₆-alkyl, —NR³(CO)-aryl, —NR³(CO)-heteroaryl, —NR³COOR⁷, —NR³(CS)NR³R⁴, whereby the heterocycloalkyl itself optionally can be interrupted by one or more nitrogen, oxygen and/or sulfur atoms, and/or optionally can be interrupted by one or more —(CO)— or —SO₂— groups in the ring, and/or optionally one or more double bonds can be contained in the ring,
   and whereby the C₃-C₆-cycloalkyl ring, and/or the C₃-C₆-heterocycloalkyl ring itself in each case optionally can be substituted in one or more places, in the same way or differently, with cyano, halogen, C₁-C₆-alkyl, C₁-C₆-hydroxyalkyl, C₁-C₆-alkoxy, C₃-C₆-cycloalkyl, or with the group —NR³R⁴ or —CO—NR³R⁴,
   or
   for the group-NR³R⁴, —NR³(CO)-aryl, —NR³(CO)-heteroaryl, or —NR³(CS)NR³R⁴,
   or
R² and R⁵ together form a C₃-C₆-heterocycloalkyl ring that is interrupted at least once by nitrogen and optionally can be interrupted in one or more places by oxygen or sulfur and/or optionally can be interrupted by one or more —(CO)— or —SO₂-groups in the ring, and/or optionally one or more double bonds can be contained in the ring,
   and/or the ring itself optionally can be substituted in one or more places, in the same way or differently, with cyano, halogen, hydroxy, C₁-C₆-alkyl, C₃-C₆-cycloalkyl, C₁-C₆-hydroxyalkyl, C₁-C₆-alkoxyalkyl, aryl or with the group —NR³R⁴,
R³ and R⁴, independently of one another, stand for hydrogen or for C₁-C₆-alkyl, C₁-C₆-alkoxy or —CO—C₁-C₆-alkyl that is optionally substituted in one or more places, in the same way or differently, with halogen, hydroxy, C₃-C₆-heterocycloalkyl, C₁-C₆-hydroxyalkoxy or with the group —NR³R⁴, whereby the heterocycloalkyl itself optionally can be interrupted by one or more nitrogen, oxygen and/or sulfur atoms, and/or optionally can be interrupted by one or more —(CO)— or —SO₂— groups in the ring, and/or optionally one or more double bonds can be contained in the ring, and whereby the C₃-C₆-heterocycloalkyl ring itself in each case optionally can be substituted in one or more places, in the same way or differently, with cyano, halogen, C₁-C₆-alkyl, C₁-C₆-hydroxyalkyl, C₁-C₆-alkoxy, C₃-C₆-cycloalkyl, or with the group —NR³R⁴ or —CO—NR³R⁴,
   or
R³ and R⁴ together form a C₃-C₆-heterocycloalkyl ring, which is interrupted at least once by nitrogen and optionally can be interrupted in one or more places by oxygen or sulfur and/or optionally can be interrupted by one or more —(CO)— or —SO₂— groups in the ring, and/or optionally one or more double bonds can be contained in the ring, and/or the heterocycloalkyl ring itself optionally can be substituted in one or more places, in the same way or differently, with C₁-C₆-alkyl, C₃-C₆-cycloalkyl, C₁-C₆-hydroxyalkyl, C₁-C₆-alkoxyalkyl, cyano, hydroxy or with the group —NR³R⁴,
R⁵ stands for C₁-C₆-alkyl, C₁-C₆-alkenyl, or C₁-C₆-alkinyl that is optionally substituted in one or more places, in the same way or differently, with halogen, hydroxy, cyano, C₁-C₆-alkoxy, C₃-C₆-cycloalkyl, C₃-C₆-heterocycloalkyl, or with the group —NR³R⁴, whereby the heterocycloalkyl itself optionally can be interrupted by one or more nitrogen, oxygen and/or sulfur atoms, and/or optionally can be interrupted by one or more —(CO)— or —SO₂— groups in the ring, and/or optionally one or more double bonds can be contained in the ring, and whereby the C₃-C₆-heterocycloalkyl ring itself in each case optionally can be substituted in one or more places, in the same way or differently, with cyano, halogen, C₁-C₆-alkyl, C₁-C₆-hydroxyalkyl, C₁-C₆-alkoxy, C₃-C₆-cycloalkyl, or with the group —NR³R⁴ or —CO—NR³R⁴,
R⁶ stands for hydroxy, C₁-C₆-alkyl, C₁-C₆-alkoxy or the group —NR³R⁴,
R⁷ stands for —(CH₂)ₙ-aryl or —(CH₂)ₙ-heteroaryl and
n stands for 1-6,
   as well as their stereoisomers, diastereomers, enantiomers and salts.

Especially preferred among them are those compounds of general formula I in which
Q stands for phenyl, naphthyl, quinolinyl, benzimidazolyl or indolyl,
A and B, independently of one another, stand for hydrogen, halogen, hydroxy, amino or nitro,
   or
   for C₁-C₃-alkyl or C₁-C₃-alkoxy that is optionally substituted in one or more places, in the same way or differently, with hydroxy, pyrrolidinyl, piperidinyl, piperazinyl or with the group —N(CH₃)₂, —N(C₂H₅)₂ or —CO(NH)(CH₂)₂N—(C₂H₅)₂, whereby pyrrolidinyl, piperidinyl or piperazinyl itself optionally can be substituted in one or more places, in the same way or differently, with C₁-C₃-alkyl, C₃-C₆-cycloalkyl, C₁-C₃-hydroxyalkyl or with the group —N(C₂H₅)₂, or
   for the group COOH, —COOCH₃, —COOC₂H₅, —CONH₂, 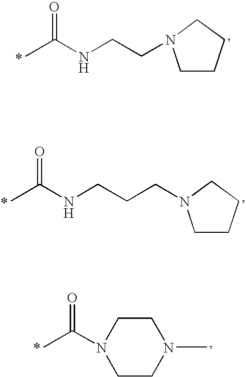
   —NH(CO)—C(CH₃)₃, —NH(CO)—(CH₂)—OCH₃, —NH(CO)—(CH₂)₂—OCH₃, —NH(CO)—(CH₂)—O(CH₂)₂—OCH₃, —NH(CO)—(CH₂)₂—N(C₂H₅)₂, 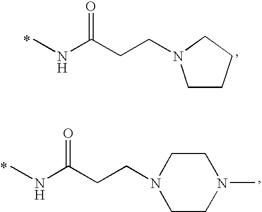
   —NH(CO)—NH(CH₂)₂—N(CH₃)₂, —NH(CO)—NH(CH₂)₂—OH, —NH(CO)—NH(CH₂)₂—O(CH₂)₂—OH, 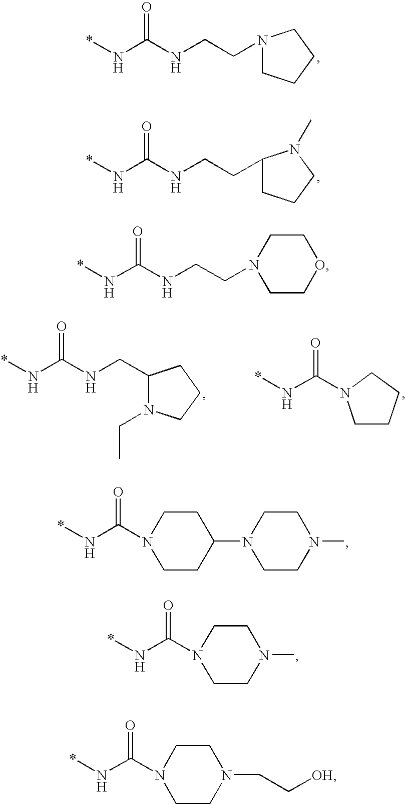
   —NH(CH₂)₂—OH, —NH(CS)NH(CH₂)₂—O(CH₂)₂—OH, 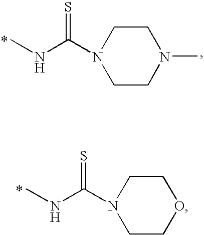
   —NHSO₂—C₁-C₆-Alkyl, —NHSO₂—CH₃, 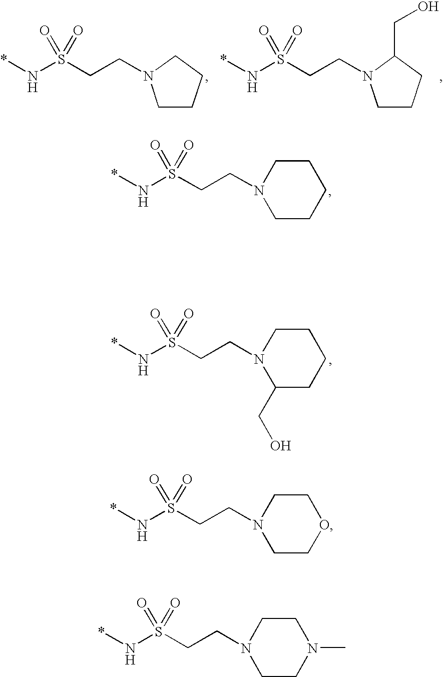
   or —SO₂—NH—(CO)—CH₃,
X stands for oxygen, —NH— or —NR⁵—,
R¹ stands for C₁-C₃-alkyl or C₃-cycloalkyl that is optionally substituted in one or more places, in the same way or differently, with fluorine, chlorine, bromine, or iodine,
R² stands for C₁-C₃-alkyl, C₁-C₃-alkoxy, C₁-C₃-alkenyl, C₁-C₃-alkinyl, C₃-C₆-cycloalkyl, isoxazolyl, piperidinyl, phenyl, pyrazolyl, pyrrolyl, (tetralin)yl or thiazolyl that is optionally substituted in one or more places, in the same way or differently, with fluorine, chlorine, bromine, iodine, hydroxy, cyano, C₁-C₆-alkyl, C₁-C₆-hydroxyalkyl, methoxy or
   with the group —S—CH₃, —COOCH₃, COOC₂H₅, —NH(CH₃), —N(CH₃)₂, —NHC(CH₃)₃, —NH(CO)—CH₃, —NH(CO)-phenyl, —NH(CO)—O—(CH₂)-phenyl, —N(CH₃)—(CS)—NH(CH₃), —N(CH₃)—(CS)—N(CH₃)₂ or
   with the following ring systems C₃-C₆-cycloalkyl, tetrahydrofuranyl, pyrrolidinyl, piperidinyl, phenyl, biphenyl, furanyl, thienyl, pyrrolyl, or pyridyl, whereby these ring systems optionally in each case themselves can be substituted in one or more places, in the same way or differently, with C₁-C₃-alkyl, cyano, fluorine, chlorine, bromine, iodine, methoxy or —CO—NH₂, or
   for the group —N(CH₃)₂, —N(CH₃)(CS)NHCH₃, —NH(CS)N(CH₃)₂, —NH(CO)-phenyl, —NH—(CH₂)—CF₃, —NH—(CH₂)₂—CF₃, —NH—(CH₂)₂—OH, —NH(CO)-pyridinyl, 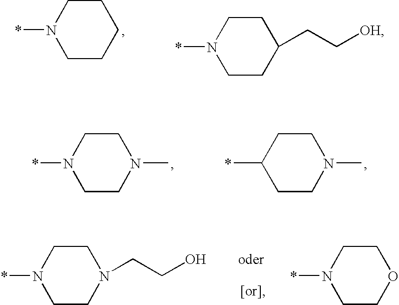
   or
R² and R together form one of the following rings: 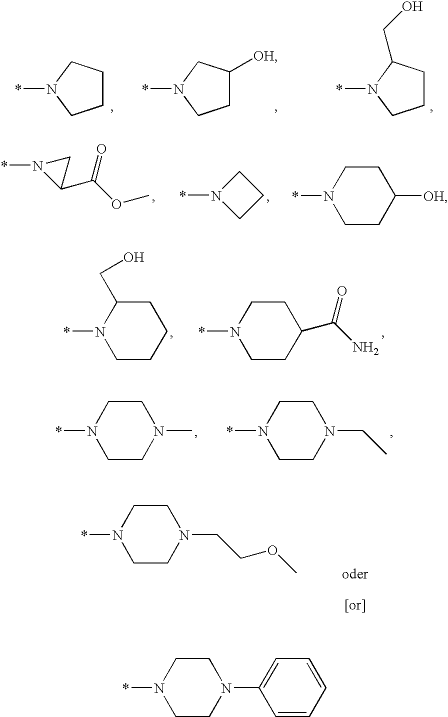
   and
R⁵ stands for C₁-C₃-alkyl, C₁-C₃-alkenyl, or C₁-C₃-alkinyl that is optionally substituted in one or more places, in the same way or differently, with halogen, hydroxy, cyano, C₁-C₆-alkoxy, C₃-C₆-cycloalkyl, C₃-C₆-heterocycloalkyl, or with the group —N(CH₃)₂,
   as well as their stereoisomers, diastereomers, enantiomers and salts.

Compounds of general formula IA 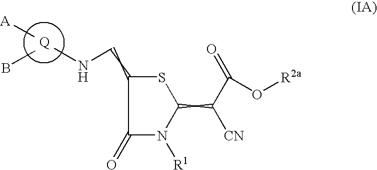
in which
Q stands for aryl or heteroaryl,
A and B, independently of one another, stand for hydrogen, halogen, hydroxy, amino or nitro
   or
   for C₁-C₃-alkyl or C₁-C₆-alkoxy that is optionally substituted in one or more places, in the same way or differently, with halogen, hydroxy, C₃-C₆-heterocycloalkyl or with the group —NR³R⁴ or —CO(NR³)-M, whereby the heterocycloalkyl itself optionally can be interrupted by one or more nitrogen, oxygen and/or sulfur atoms, and/or optionally can be interrupted by one or more —(CO)— or —SO₂— groups in the ring, and/or optionally one or more double bonds can be contained in the ring, and/or the ring itself optionally can be substituted in one or more places, in the same way or differently, with C₁-C₆-alkyl, C₃-C₆-cycloalkyl, C₁-C₆-hydroxyalkyl or with the group —NR³R⁴, or
   for —NR³ (CO)-L, —NR³(Co)—NR³-L, —COR⁶, —CO(NR³)-M, —NR³(CS)NR³R^{e}, —NR³SO₂-M, —SO₂—NR³R⁴ or —SO₂(NR³)-M,
L stands for C₁-C₆-alkyl or heteroaryl that is optionally substituted in one or more places, in the same way or differently, with C₁-C₆-hydroxyalkoxy, C₁-C₆-alkoxyalkoxy, C₃-C₆-heterocycloalkyl or with the group —NR³R⁴, whereby the heterocycloalkyl itself optionally can be interrupted by one or more nitrogen, oxygen and/or sulfur atoms, and/or optionally can be interrupted by one or more —(CO)— or —SO₂— groups in the ring, and/or optionally one or more double bonds can be contained in the ring, and/or the ring itself optionally can be substituted in one or more places, in the same way or differently, with C₁-C₆-alkyl, C₃-C₆-cycloalkyl, C₁-C₆-hydroxyalkyl or with the group —NR³R⁴,
M stands for C₁-C₆-alkyl that is optionally substituted in one or more places, in the same way or differently, with the group —NR³R⁴ or C₃-C₆-heterocycloalkyl,
R¹ stands for C₁-C₄-alkyl, C₃-cycloalkyl, allyl or propargyl that is optionally substituted in one or more places, in the same way or differently, with halogen,
R^{2a} stands for allyl or propargyl,
R³ and R⁴, independently of one another, stand for hydrogen or for C₁-C₆-alkyl, C₁-C₆-alkoxy, —CO—C₁-C₆-alkyl or aryl that is optionally substituted in one or more places, in the same way or differently, with halogen, hydroxy, C₃-C₆-heterocycloalkyl, C₁-C₆-hydroxyalkoxy or with the group —NR³R⁴, whereby the heterocycloalkyl itself optionally can be interrupted by one or more nitrogen, oxygen and/or sulfur atoms, and/or optionally can be interrupted by one or more —(CO)— or —SO₂— groups in the ring, and/or optionally one or more double bonds can be contained in the ring, and whereby the C₃-C₆-heterocycloalkyl ring itself in each case optionally can be substituted in one or more places, in the same way or differently, with cyano, halogen, C₁-C₆-alkyl, C₁-C₆-hydroxyalkyl, C₁-C₆-alkoxy, C₃-C₆-cycloalkyl, or with the group —NR³R⁴ or —CO—NR³R⁴,
   or
R³ and R⁴ together form a C₃-C₆-heterocycloalkyl ring, which is interrupted at least once by nitrogen and optionally can be interrupted in one or more places by oxygen or sulfur, and/or optionally can be interrupted by one or more —(CO)— or —SO₂— groups in the ring, and/or optionally one or more double bonds can be contained in the ring,
   and/or the heterocycloalkyl ring itself optionally can be substituted in one or more places, in the same way or differently, with C₁-C₆-alkyl, C₃-C₆-cycloalkyl, C₁-C₆-hydroxyalkyl, C₁-C₆-alkoxyalkyl, cyano, hydroxy or with the group —NR³R⁴, and
R⁶ stands for hydroxy, C₁-C₆-alkyl, C₁-C₆-alkoxy or the group —NR³R⁴,
   as well as their solvates, hydrates, stereoisomers, diastereomers, enantiomers and salts, are another subject of this invention.

These compounds exhibit an allyl ester or a propargyl ester in contrast to the compounds of general formula I. These compounds also inhibit kinases of the polo family and are better suitable for cleavage into the free acid and thus for the production of compounds of general formula I in particular because of the presence of allyl ester.

Preferred are those compounds of general formula IA in which
Q stands for phenyl, quinolinyl, indolyl or naphthyl,
A and B, independently of one another, stand for hydrogen or halogen,
   or
   for C₁-C₃-alkyl or C₁-C₆-alkoxy that is optionally substituted in one or more places, in the same way or differently, with halogen, hydroxy or with the group —NC₁-C₆-alkyl)₂ or —CO(NH)-M,
   or
   for —NH(CO)-L, —NH(CO)—NH-L, —COR⁶, —CO(NH)-M, —CO(NCH₃)-M, SO₂(NH)-M or —SO₂(NCH₃)-M,
L stands for C₁-C₆-alkyl that is optionally substituted in one or more places, in the same way or differently, with pyrrolidinyl,
M stands for C₁-C₆-alkyl that is optionally substituted in one or more places, in the same way or differently, with the group —N(C₁-C₆-alkyl)₂ or pyrrolidinyl,
R¹ stands for C₁-C₃-alkyl,
R^{2a} stands for allyl or propargyl, and
R⁶ stands for hydroxy, C₁-C₆-alkyl or C₁-C₆-alkoxy,
   as well as their solvates, hydrates, stereoisomers, diastereomers, enantiomers and salts.

In particular, preferred compounds are the compounds of production examples 77, 104, 105, 106, 107, 117, 119, 121, 123-131, 133, 135, 137, and 140.

Production examples 1 to 75, as well as their solvates, hydrates, stereoisomers, diastereomers, enantiomers and salts, represent another subject of the invention. These compounds are distinguished from those of general formula I by the presence of an ester radical instead of an amide bond. These compounds are suitable for inhibiting kinases of the polo family. In addition, these compounds are suitable as intermediate products for the production of compounds of general formula I.

In particular R¹ as C₁-C₄-alkyl or C₃-cycloalkyl that is optionally substituted with halogen, as well as the secondary amine at Q 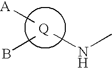
represent essential features of the compounds according to the invention.

In particular, also those uses of the compounds of general formulas IIA, IIB, IIIA, IIIB, IVA, and IVB as well as compounds of general formula V, as intermediate products for the production of the compounds of general formula I, represent additional subjects of the invention:

Uses of the compounds of general formula IIA or IIB 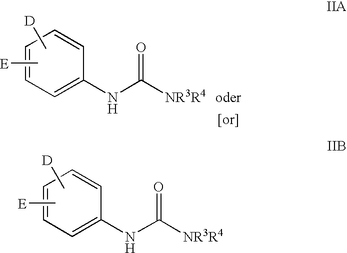
in which D stands for the group —NO₂, —NH₂ or —NH(CO)OC(CH₃)₃ and E stands for C₁-C₆-alkoxy or halogen, and R³ and R⁴ have the meaning that is described in general formula I, as intermediate products for the production of the substances of general formula I according to the invention.

Uses of the compounds of general formula IIIA or IIIB 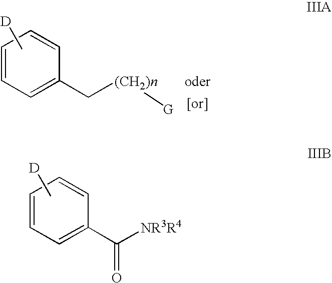
in which D stands for the group —NO₂, —NH₂ or —NH(CO)OC(CH₃)₃ and G stands for the group —NR³R⁴, and R³, R⁴ and n have the meaning that is described in general formula I, as intermediate products for the production of the substances of general formula I according to the invention.

Uses of the compounds of general formula IVA or IVB 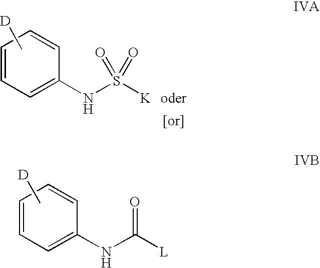
in which D stands for the group —NO₂, —NH₂ or —NH(CO)OC(CH₃)₃ and K stands for C₁-C₆-alkyl or C₁-C₆-alkenyl that is optionally substituted with the group —NR³R⁴ and L stands for C₁-C₆-alkyl or C₁-C₆-alkenyl that is optionally substituted with C₁-C₆-alkoxy, C₁-C₆-alkoxy-C₁-C₆-alkoxy or the group —NR³R⁴, and R³ and R⁴ have the meaning that is described in general formula I, as intermediate products for the production of substances of general formula I according to the invention.

Compounds of general formula V 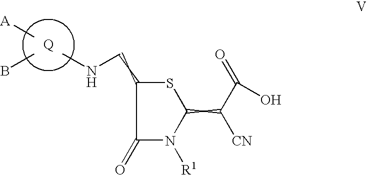
in which Q, A, B and R¹ have the meaning that is described in general formula I, as intermediate products for the production of the substances of general formula I according to the invention, with the proviso of cyano-[3-ethyl-4-oxo-5-[1-phenylamino-meth-(E/Z)-ylidene]-thiazolidin-(2-(E or Z))-ylidene]-acetic acid, do not fall under general formula V:

To use the compounds of general formula I according to the invention as pharmaceutical agents, the latter are brought into the form of a pharmaceutical preparation, which in addition to the active ingredient for enteral or parenteral administration contains suitable pharmaceutical, organic or inorganic inert support media, such as, for example, water, gelatin, gum arabic, lactose, starch, magnesium stearate, talc, vegetable oils, polyalkylene glycols, etc. The pharmaceutical preparations can be present in solid form, for example as tablets, coated tablets, suppositories, or capsules, or in liquid form, for example as solutions, suspensions, or emulsions. Moreover, they optionally contain adjuvants, such as preservatives, stabilizers, wetting agents or emulsifiers; salts for changing the osmotic pressure or buffers.

These pharmaceutical preparations are also subjects of this invention.

For parenteral administration, especially injection solutions or suspensions, especially aqueous solutions of active compounds in polyhydroxyethoxylated castor oil, are suitable.

As carrier systems, surface-active adjuvants, such as salts of bile acids or animal or plant phospholipids, but also mixtures thereof, as well as liposomes or their components can also be used.

For oral administration, especially tablets, coated tablets or capsules with talc and/or hydrocarbon vehicles or binders, such as, for example, lactose, corn or potato starch, are suitable. The administration can also be carried out in liquid form, such as, for example, as a juice, to which optionally a sweetener is added.

Enteral, parenteral and oral administrations are also subjects of this invention.

The dosage of the active ingredients can vary depending on the method of administration, age and weight of the patient, type and severity of the disease to be treated and similar factors. The daily dose is 0.5-1000 mg, preferably 50-200 mg, whereby the dose can be given as a single dose to be administered once or divided into two or more daily doses.

Subjects of this invention also include the use of compounds of general formula I for the production of a pharmaceutical agent for treating cancer, auto-immune diseases, cardiovascular diseases, chemotherapy agent-induced alopecia and mucositis, infectious diseases, nephrological diseases, chronic and acute neurodegenerative diseases and viral infections, whereby cancer is defined as solid tumors and leukemia; auto-immune diseases are defined as psoriasis, alopecia and multiple sclerosis; cardiovascular diseases are defined as stenoses, arterioscleroses and restenoses; infectious diseases are defined as diseases that are caused by unicellular parasites; nephrological diseases are defined as glomerulonephritis; chronic neurodegenerative diseases are defined as Huntington's disease, amyotrophic lateral sclerosis, Parkinson's disease, AIDS dementia and Alzheimer's disease; acute neurodegenerative diseases are defined as ischemias of the brain and neurotraumas; and viral infections are defined as cytomegalic infections, herpes, hepatitis B or C, and HIV diseases.

Subjects of this invention also include pharmaceutical agents for treating the above-cited diseases, which contain at least one compound according to general formula I, as well as pharmaceutical agents with suitable formulation substances and vehicles.

The compounds of general formula I according to the invention are, i.a., excellent inhibitors of the polo-like kinases, such as Plk1, Plk2, Plk3, and Plk4.

If the production of the starting compounds is not described, the latter are known or can be produced analogously to known compounds or to processes that are described here. It is also possible to perform all reactions that are described here in parallel reactors or by means of combinatory operating procedures.

The isomer mixtures can be separated into the isomers, such as, e.g., into the enantiomers, diastereomers or E/Z isomers, according to commonly used methods, such as, for example, crystallization, chromatography or salt formation, if the isomers are not in a state of equilibrium with one another.

The production of the salts is carried out in the usual way by a solution of the compound of formula I being mixed with the equivalent amount of or excess base or acid, which optionally is in solution, and the precipitate being separated or the solution being worked up in the usual way.

Production of the Compounds According to the Invention

The following examples explain the production of the compounds according to the invention, without the scope of the claimed compounds being limited to these examples.

The compounds of general formula I or IA according to the invention can be produced according to the following general diagrams of the process: 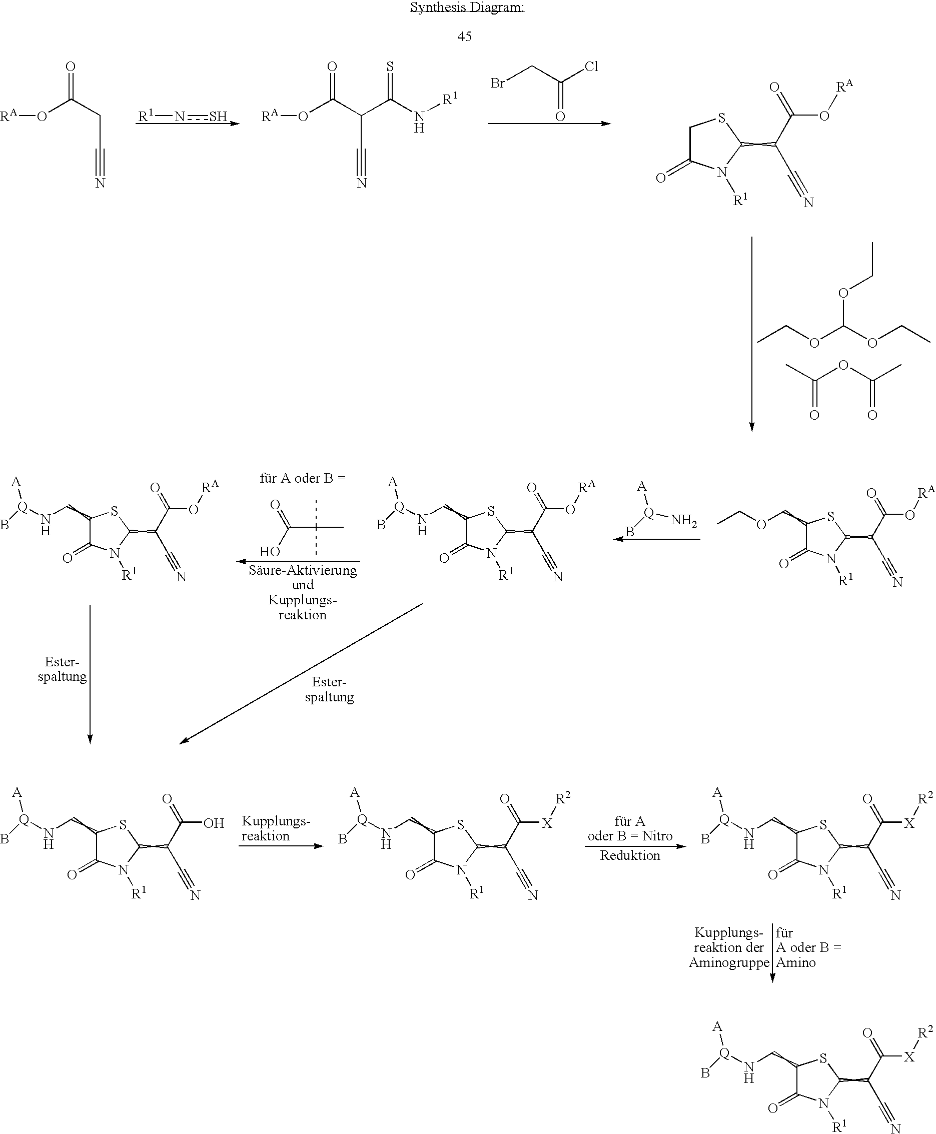
R^{A}=Ethyl, propyl, allyl, benzyl
R¹, R², A, B and Q have the meaning that is indicated in general formula I
[Key to Synthesis Diagram:]
für A oder B=for A or B
Saüre-Aktivierung und Kupplungsreaktion=Acid activation and coupling reaction
Esterspaltung=ester cleavage
Reduktion=reduction
Kupplungsreaktion der Aminogruppe=Coupling reaction of the amino group 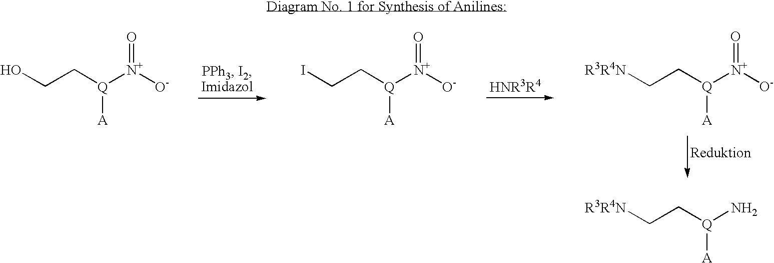
whereby A, Q, R³ and R⁴ have the meaning that is indicated in general formula I.
[Key to Diagram No. 1:]
Imidazol=Imidazole
Reduktion=Reduction 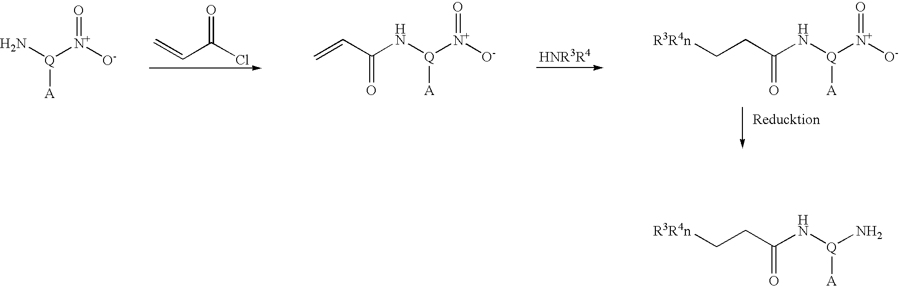
whereby A, Q, R³ and R⁴ have the meaning that is indicated in general formula I.
[Key to Diagram No. 2:]
Reduktion=Reduction 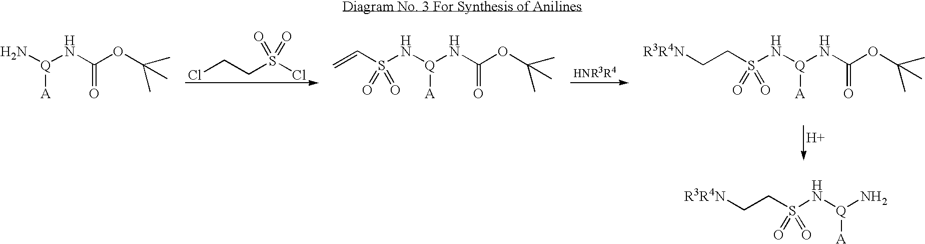
whereby A, Q, R³ and R⁴ have the meaning that is indicated in general formula I. 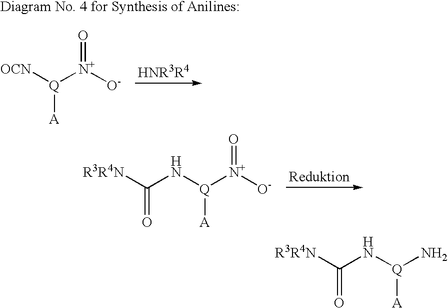
whereby A, Q, R³ and R⁴ have the meaning that is indicated in general formula I.
[Key to Diagram No. 4:]
Reduktion=Reduction 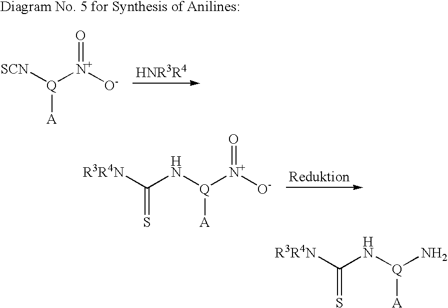
whereby A, Q, R³ and R⁴ have the meaning that is indicated in general formula I.
[Key to Diagram No. 5:]
Reduktion=Reduction 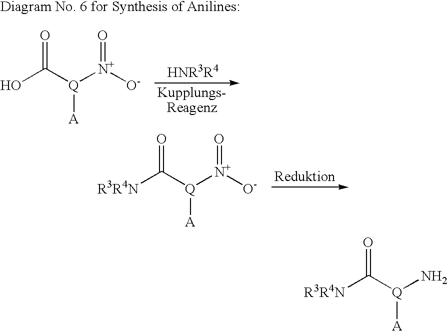
whereby A, Q, R³ and R⁴ have the meaning that is indicated in general formula I.
[Key to Diagram No. 6:]
Kupplungsreagenz=Coupling reagent
Reduktion=Reduction 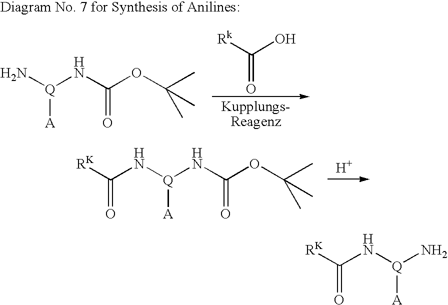
R^{K}=C¹-C⁶ alkyl or —(CH₂), C₁-C₆— alkoxy or —(CH₂)ₙ C₁-C₆— alkoxyalkoxy whereby A, Q, R³ and R⁴ have the meaning that is indicated in general formula I.
[Key to Diagram No. 7:]
Kupplungsreagenz=Coupling reagent 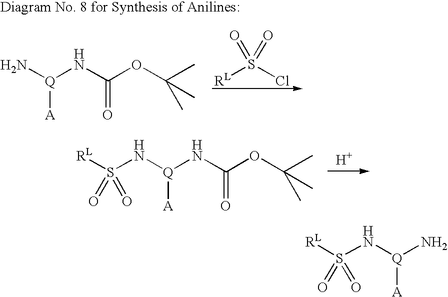
R^{L}=C¹-C⁶ alkyl
whereby A and Q have the meaning that is indicated in general formula I. 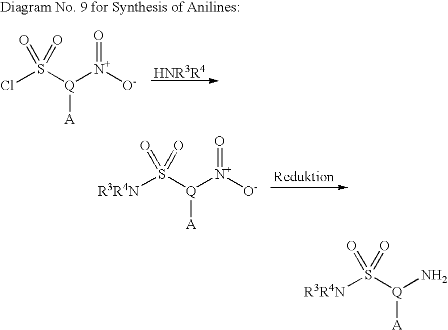
whereby A, Q, R³ and R⁴ have the meaning that is indicated in general formula I.
[Key to Diagram No. 9:]
Reduktion=Reduction 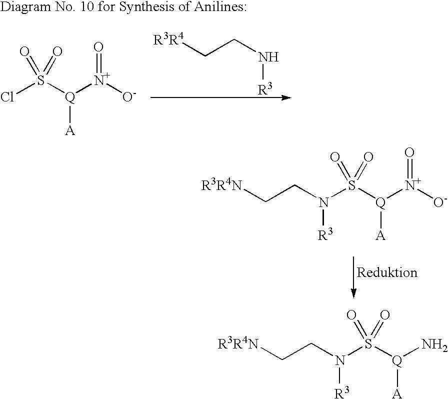
whereby A, Q, R³ and R⁴ have the meaning that is indicated in general formula I.
[Key to Diagram No. 10:]
Reduktion=Reduction 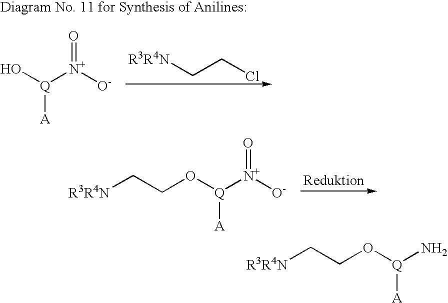
whereby A, Q, R³ and R⁴ have the meaning that is indicated in general formula I.
[Key to Diagram No. 11:]
Reduktion=Reduction 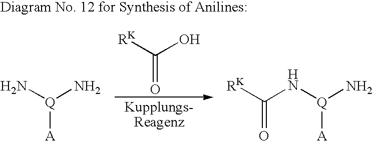
R^{K}=C¹-C⁶ alkyl or —(CH₂)ₙ C₁-C₆-alkoxy or —(CH₂)ₙ C₁-C₆-alkoxyalkoxy whereby A and Q have the meaning that is indicated in general formula I.
[Key to Diagram No. 12:]
Kupplungsreagenz=Coupling reagent 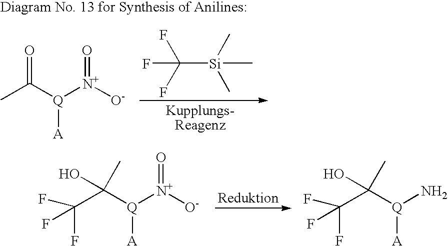
whereby A and Q have the meaning that is indicated in general formula I.
[Key to Diagram No. 13:]
Kupplungsreagenz=Coupling reagent
Reduktion=Reduction 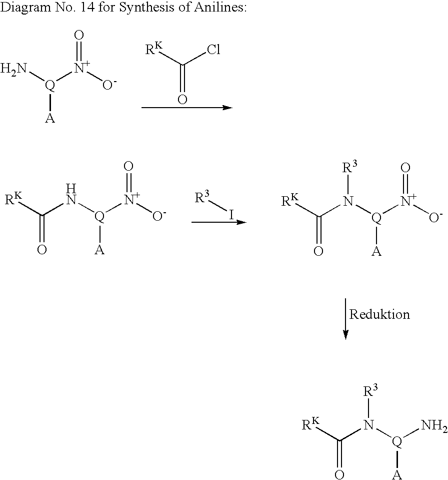
R^{K}=C¹-C⁶ alkyl or —(CH₂)ₙ C₁-C₆— alkoxy or —(CH₂), C₁-C₆-alkoxyalkoxy whereby A, Q and R³ have the meaning that is indicated in general formula I.
[Key to Diagram No. 14:]
Reduktion=Reduction
Synthesis of Intermediate Compounds
Production of the intermediate compounds (INT) that preferably can be used for the production of the thiazolidinone compounds according to the invention.

### EXAMPLE INT1

### N-(3-Amino-phenyl)-2,2-dimethyl-propionamide

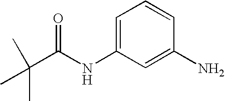

5.0 g of the 1,3-diaminobenzene is dissolved in 50 ml of dichloromethane and mixed at 0° C. with 24 ml of diisopropylethylamine and 10.4 ml of pivalic acid anhydride. It is stirred for 2 hours at 0° C. and for 18 hours at room temperature. The reaction mixture is mixed with semi-saturated sodium bicarbonate solution and extracted with ethyl acetate. The organic solution is washed with saturated sodium chloride solution, dried on sodium sulfate, concentrated by evaporation, and after purification by chromatography on silica gel, 5.7 g of the title compound is obtained.

1H-NMR (DMSO-d6): δ=1.20 (s, 9H); 4.98 (s, 2H); 6.24 (d, 1H); 6.70 (d, 1H); 6.83-6.96 (m, 2H) ppm.

### EXAMPLE INT2

### 1-(2-Iodo-ethyl)-4-nitro-benzene

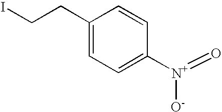

15 g of 4-nitrophenylethanol, 28.1 g of triphenylphosphine and 9.2 g of imidazole are dissolved in 500 ml of THF, mixed in portions with 27.77 g of iodine and stirred for 2 hours at room temperature. The reaction mixture is mixed with ammonium chloride solution and extracted with dichloromethane. The organic phase is washed in succession with sodium thiosulfate solution and water and dried on sodium sulfate. After purification by chromatography on silica gel, 23.22 g of the title compound is obtained.

1H-NMR (DMSO-d6): δ=3.30 (t, 2H); 3.54 (t, 2H); 7.57 (d, 2H); 8.18 (d, 2H) ppm.

### EXAMPLE INT3

### 1-[2-(4-Nitro-phenyl)-ethyl]-pyrrolidine

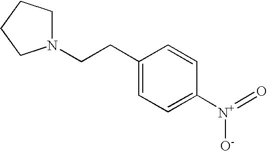

8 g of the compound that is described under Example INT2), 26.4 g of potassium carbonate and 3.6 ml of pyrrolidine are dissolved in 20 ml of DMF and stirred for 5 hours at room temperature. The solvent is condensed under high vacuum, the residue is taken up in ethyl acetate and washed three times with water. The organic phase is dried on sodium sulfate. After purification by chromatography on silica gel, 5.6 g of the title compound is obtained.

1H-NMR (DMSO-d6): δ=1.68 (m, 4H); 2.48 (m, 4H); 2.67 (t, 2H); 2.89 (t, 2H); 7.52 (d, 2H); 8.13 (d, 2H) ppm.

### EXAMPLE INT4

### 4-(2-Pyrrolidin-1-yl-ethyl)-phenylamine

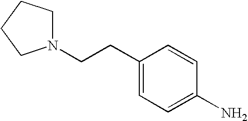

5.67 g of the compound that is described under Example INT3) is dissolved in 500 ml of ethanol and mixed with 1 g of palladium on carbon (10%). It is stirred for 2 hours under hydrogen atmosphere at room temperature. After filtration on diatomaceous earth and after the solvent is condensed in a rotary evaporator, 4.8 g of the title compound is obtained.

1H-NMR (DMSO-d6): δ=1.67 (m, 4H); 2.31-2.60 (m, 8H); 4.81 (s, 2H); 6.48 (d, 2H); 6.84 (d, 2H) ppm.

### EXAMPLE INT5

### 1-Methyl-4-[2-(4-nitro-phenyl)-ethyl]-piperazine

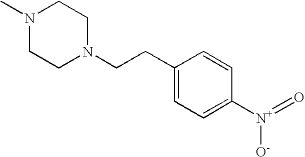

5 g of the compound that is described under Example INT2), 6.2 ml of triethylamine and 2.4 ml of N-methylpiperazine are dissolved in 20 ml of tetrahydrofuran and stirred for 3 hours under reflux. Another 0.6 ml of N-methylpiperazine is added, and it is stirred for another 3 hours under reflux. The solvent is condensed in a rotary evaporator, the residue is taken up in ethyl acetate and washed with water. The organic phase is dried on sodium sulfate. After purification by chromatography on silica gel, 1.6 g of the title compound is obtained.

1H-NMR (DMSO-d6): δ=2.13 (s, 3H); 2.20-2.48 (m, 8H); 2.54 (t, 2H); 2.87 (t, 2H); 7.51 (d, 2H); 8.13 (d, 2H) ppm.

### EXAMPLE INT6

### 4-[2-(4-Methyl-piperazin-1-yl)-ethyl]-phenylamine

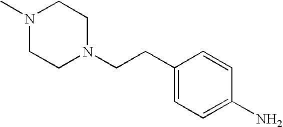

6.37 g of the compound that is described under Example INT5) is dissolved in 500 ml of ethanol and mixed with 1.1 g of palladium on carbon (10%). It is stirred for 2 hours under hydrogen atmosphere at room temperature. After filtration on diatomaceous earth and after the solvent is condensed in a rotary evaporator, 5.6 g of the title compound is obtained.

1H-NMR (DMSO-d6): δ=2.15 (s, 3H); 2.20-2.59 (m, 12H); 4.80 (s, 2H); 6.48 (d, 2H); 6.83 (d, 2H) ppm.

### EXAMPLE INT7

### {1-[2-(4-Nitro-phenyl)-ethyl]-piperidin-4-yl}-methanol

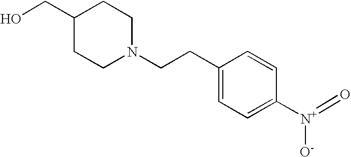

8 g of the compound that is described under Example INT2), 26.4 g of potassium carbonate and 5.0 g of 4-hydroxymethylpiperidine are dissolved in 20 ml of DMF and stirred for 18 hours at room temperature. The solvent is condensed under high vacuum, the residue is taken up in ethyl acetate and washed three times with water. The organic phase is dried on sodium sulfate. After purification by chromatography on silica gel, 5.56 g of the title compound is obtained.

1H-NMR (DMSO-d6): δ=0.99-1.16 (m, 2H); 1.21-1.41 (m, 1H); 1.61 (d, 2H); 1.90 (t, 2H); 2.54 (t, 2H); 2.81-2.98 (m, 4H); 3.23 (d, 2H); 4.40 (s, 1H); 7.50 (d, 2H); 8.13 (d, 2H) ppm.

### EXAMPLE INT8

### {1-[2-(4-Amino-phenyl)-ethyl]-piperidin-4-yl}-methanol

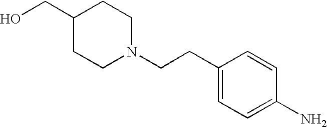

6.56 g of the compound that is described under Example INT7) is dissolved in 500 ml of ethanol and mixed with 1.1 g of palladium on carbon (10%). It is stirred for 4 hours under hydrogen atmosphere at room temperature. After filtration on diatomaceous earth and after the solvent is condensed in a rotary evaporator, 4.67 g of the title compound is obtained.

1H-NMR (DMSO-d6): δ=0.99-1.20 (m, 2H); 1.20-1.41 (m, 1H); 1.61 (d, 2H); 1.87 (t, 2H); 2.36 (t, 2H); 2.50-2.60 (m, 2H); 2.88 (d, 2H); 3.23 (t, 2H); 4.40 (s, 1H); 4.80 (s, 2H); 6.47 (d, 2H); 6.84 (d, 2H) ppm.

### EXAMPLE INT9

### (4-Ethenesulfonylamino-phenyl)-carbamic acid tert-butyl ester

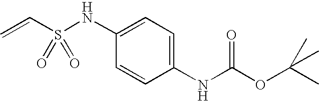

2.0 g of (4-aminophenyl)-carbamic acid (tert)butyl ester is dissolved in 60 ml of tetrahydrofuran, mixed with 6.74 ml of triethylamine and with 1.0 ml of 2-chloroethanesulfonic acid chloride and stirred for 2 hours at room temperature. The reaction mixture is mixed with water and extracted with ethyl acetate. The organic solution is washed in succession with 4N hydrochloric acid, with semi-saturated sodium bicarbonate solution and with saturated sodium chloride solution, dried on sodium sulfate, concentrated by evaporation, and after recrystallization from ethanol/dichloromethane (1:3), 1.45 g of the title compound is obtained.

1H-NMR (DMSO-d6): δ=1.47 (s, 9H); 5.97 (d, 1H); 6.01 (d, 1H); 6.70 (dd, 1H); 7.03 (d, 2H); 7.35 (d, 2H); 9.28 (s, 1H); 9.70 (s, 1H) ppm.

### EXAMPLE INT10

### [4-(2-Morpholin-4-yl-ethanesulfonylamino)-phenyl]-carbamic acid tert-butyl ester

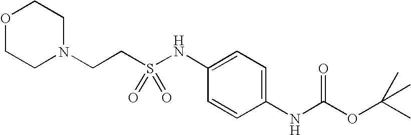

107 mg of the compound that is described under Example INT9) is dissolved in 5 ml of tetrahydrofuran, mixed with 0.25 ml of triethylamine and 71 μl of morpholine and stirred under reflux for 24 hours. The reaction mixture is mixed with water and extracted with ethyl acetate. The organic solution is washed with saturated sodium chloride solution, dried on sodium sulfate, concentrated by evaporation, and, after purification by chromatography on silica gel, 62 mg of the title compound is obtained.

1H-NMR (DMSO-d6, stored with K2CO3): δ=1.47 (s, 9H); 2.30 (m, 4H); 2.63 (t, 2H); 3.14 (t, 2H); 3.50 (m, 4H); 7.08 (d, 2H); 7.37 (d, 2H); 9.25 (s, 1H); 9.52 (s, 1H) ppm.

### EXAMPLE INT11

### [4-(2-Methoxyacetylamino)-phenyl]-carbamic acid tert-butyl ester

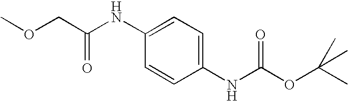

200 mg of (4-aminophenyl)-carbamic acid (tert)butyl ester is dissolved in 5 ml of tetrahydrofuran, mixed with 0.63 ml of triethylamine and 0.16 ml of methoxyacetyl chloride and stirred for 2 hours at room temperature. The reaction mixture is mixed with semi-saturated sodium bicarbonate solution and extracted with ethyl acetate. The organic solution is washed with saturated sodium chloride solution, dried on sodium sulfate, concentrated by evaporation, and after purification by chromatography on silica gel, 211 mg of the title compound is obtained.

1H-NMR (DMSO-d6, stored with K2CO3): δ=1.48 (s, 9H); 3.38 (s, 3H); 3.97 (s, 2H); 7.37 (d, 2H); 7.52 (d, 2H); 9.25 (s, 1H); 9.61 (s, 1H) ppm.

### EXAMPLE INT12)

### N-(4-Nitrophenyl)-acrylamide

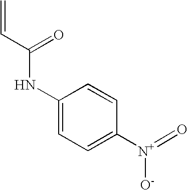

First, 61 ml of triethylamine and then 14.6 ml of acrylic acid chloride are added to a solution of 20 g of 4-nitroaniline in 200 ml of THF. The mixture is stirred for 4 hours at room temperature, mixed with sodium chloride solution and extracted with ethyl acetate. The crude product that is obtained after the solvent is evaporated is recrystallized (dichloromethane). 18.5 g of the title compound is obtained.

¹H-NMR (CDCl₃): δ=5.87 (1H); 6.34 (1H); 6.47 (1H); 7.92 (2H); 8.23(2H) ppm.

### EXAMPLE INT13)

### 3-(4-Methyl-piperazin-1-yl)-N-(4-nitro-phenyl)-propionamide

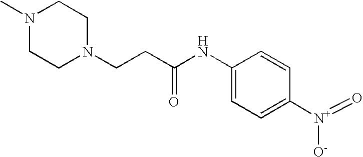

First, 31.2 ml of triethylamine and then 11.7 ml of 1-methylpiperazine are added to a solution of 8.6 g of N-(4-nitrophenyl)-acrylamide in 225 ml of THF. The mixture is stirred under reflux for 15 hours and evaporated to the dry state in a rotary evaporator. After dichloromethane is added, it is extracted with sodium bicarbonate solution and sodium chloride solution, dried on sodium sulfate, and the solvent is evaporated. The crude product that is obtained is recrystallized (ethyl acetate). 8.0 g of the title compound is obtained.

Molar mass=292.341; MS (ESI): [M+1]+=293.

### EXAMPLE INT14)

### N-(4-Amino-phenyl)-3-(4-methyl-piperazin-1-yl)-propionamide

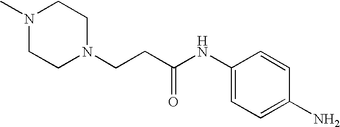

A mixture of 8.6 g of N-(4-nitrophenyl)-acrylamide and 0.8 g of palladium on carbon (10%) in 150 ml of ethanol was stirred in a hydrogen atmosphere for 5 hours at room temperature. Then, the mixture was filtered on Celite, and the solvent was evaporated. 7.0 g of the title compound was obtained.

¹H-NMR (CDCl₃): δ=2.14 (3H); 2.19-2.52 (10H) 2.58 (2H); 4.92 (2H); 6.71 (2H); 7.05 (2H); 7.83 (1H); ppm.

### EXAMPLE INT15

### N-(3-Nitro-phenyl)-acrylamide

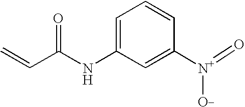

Analogously to Example INT12), 18.5 g of the title compound is obtained from 20 g of 3-nitroaniline, 61 ml of triethylamine and 14.6 ml of acrylic acid chloride, after purification by recrystallization from dichloromethane.

1H-NMR (DMSO-d6): δ=5.84 (dd, 1H); 6.32 (dd, 1H); 6.45 (dd, 1H); 7.62 (t, 1H); 7.89-8.02 (m, 2H); 8.70 (s, 1H); 9.6-11.0 (b, 1H) ppm.

### EXAMPLE INT16

### N-(3-Nitro-phenyl)-3-pyrrolidin-1-yl-propionamide

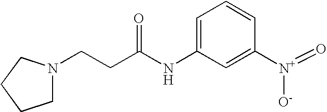

Analogously to Example INT13), after purification by chromatography on silica gel, 5.52 g of the title compound is obtained from 5.0 g of the compound that is produced under Example INT15), 18.2 ml of triethylamine and 2.56 ml of pyrrolidine.

1H-NMR (DMSO-d6): δ=1.60-1.76 (m, 4H); 2.38-2.58 (m, 6H); 2.72 (t, 2H); 7.60 (t, 1H); 7.85-7.93 (m, 2H); 8.64 (s, 1H); 10.56 (s, 1H) ppm.

### EXAMPLE INT17

### N-(3-Amino-phenyl)-3-pyrrolidin-1-yl-propionamide

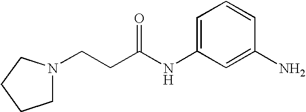

5.5 g of the compound that is described under Example INT16) is dissolved in 200 ml of ethanol and mixed with 450 mg of palladium on carbon (10%). It is stirred for 4 hours under hydrogen atmosphere at room temperature. After filtration on diatomaceous earth, and after the solvent is condensed in a rotary evaporator, 4.8 g of the title compound is obtained.

1H-NMR (DMSO-d6): δ=1.61-1.75 (m, 4H); 2.34-2.53 (m, 6H); 2.68 (t, 2H); 5.02 (s, 2H); 6.21 (d, 1H); 6.55 (d, 1H); 6.82-6.94 (m, 2H); 9.78 (s, 1H) ppm.

### EXAMPLE INT18

### 3-Nitro-N-(3-pyrrolidin-1-yl-propyl)-benzamide

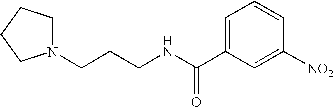

500 mg of 4-nitrobenzoic acid is dissolved in 20 ml of dimethylformamide, mixed with 370 μl of triethylamine, 342 mg of N-(3-aminopropyl)-pyrrolidine and 866 mg of TBTU, and it is stirred for 20 hours at room temperature. The reaction mixture is mixed with semi-saturated sodium bicarbonate solution, and extracted with dichloromethane. The organic solution is washed with saturated sodium chloride solution, dried on sodium sulfate, concentrated by evaporation, and after purification by chromatography on silica gel, 502 mg of the title compound is obtained.

1H-NMR (DMSO): δ=1.84 (m, 6H), 2.63 (m, 4H), 2.78 (m, 2H), 7.61 (m, 1H), 8.22 (dd, 1H), 8.32 (dd, 1H), 8.53 (m, 1H), 9.41 (s, 1H) ppm.

### EXAMPLE INT19

### 3-Amino-N-(3-pyrrolidin-1-yl-propyl)-benzamide

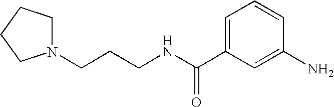

1 g of the compound that is described under Example INT18) is dissolved in 50 ml of THF and mixed with 1 g of Raney nickel. It is stirred for 3 hours under hydrogen atmosphere at room temperature. After filtration on diatomaceous earth and after the solvent is condensed in a rotary evaporator, 810 mg of the title compound is obtained.

1H-NMR (DMSO d6): δ=1.79 (m, 6H), 2.57 (m, 4H), 2.69 (m, 2H), 3.55 (m, 2H), 3.73 (s, 2H), 6.76 (dd, 1H), 7.02 (m, 1H), 7.17 (m, 2H), 8.52 (s, 1H) ppm.

### EXAMPLE INT20

### Pyrrolidine-1-carboxylic acid (4-nitro-phenyl)-amide

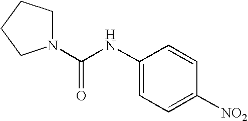

1 g of para-nitrophenylisocyanate is dissolved in 10 ml of acetonitrile and slowly mixed at room temperature with pyrrolidine (1.51 ml). It is stirred overnight at room temperature, the solvent is distilled off in a rotary evaporator, and the residue is recrystallized from ethanol. 1.1 g of product is obtained.

1H-NMR (DMSO d6): δ=1.82 (m, 4H), 3.48 (m, 4H), 7.79 (d, 2H), 8.12 (d, 2H), 8.80 (s, 1H) ppm.

### EXAMPLE INT21

### Pyrrolidine-1-carboxylic acid (4-amino-phenyl)-amide

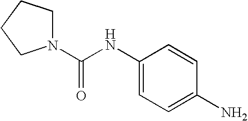

1 g of the compound that is described under Example INT20) is dissolved in 50 ml of THF and mixed with 1 g of Raney nickel. It is stirred for 3 hours under hydrogen atmosphere at room temperature. After filtration on diatomaceous earth and after the solvent is condensed in a rotary evaporator, 790 mg of the title compound is obtained.

1H-NMR (DMSO d6): δ=1.80 (m, 4H), 3.28 (m, 4H), 4.68 (s, 2H), 6.42 (d, 2H), 7.05 (d, 2H), 7.61 (s, 1H) ppm.

### EXAMPLE INT22

### N-(3-Amino-5-chloro-phenyl)-2,2-dimethyl-propionamide (2056-1)

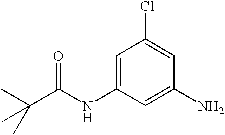

5.0 g of 5-chloro-1,3-diaminobenzene is dissolved in 50 ml of dichloromethane and 5 ml of dimethylformamide and mixed at 0° C. with 18.5 ml of diisopropylethylamine and 8.5 ml of pivalic acid anhydride. It is stirred for one hour at 0° C. and for 5 hours at room temperature. The reaction mixture is mixed with semi-saturated sodium bicarbonate solution and extracted with a mixture that consists of ethyl acetate and hexane (1:3). The organic solution is washed with saturated sodium chloride solution, dried on sodium sulfate, concentrated by evaporation, and after purification by chromatography on silica gel, 2.5 g of the title compound is obtained.

1H-NMR (DMSO-d6): (DMSO-d6): δ=5.37 (s,b, 2H); 6.28 (s,b, 1H); 6.88 (s,b, 1H); 7.48 (s, 1H); 9.00 (s, 1H) ppm.

### EXAMPLE INT23

### Ethyl-(5-nitro-pyridin-2-yl)-amine

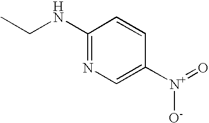

395 mg of 2-chloro-5-nitro-pyridine and 2.5 ml of a 1 M solution of ethylamine in tetrahydrofuran are dissolved in 10 ml of DMSO and stirred for 4 hours at 50° C. The reaction mixture is mixed with ethyl acetate and washed three times with semi-saturated sodium bicarbonate solution. The organic phase is dried on sodium sulfate. After purification by chromatography on silica gel, 430 mg of the title compound is obtained.

1H-NMR (DMSO-d6): δ=1.17 (t, 3H); 3.40 (m, 2H); 6.53 (d, 1H); 8.00-8.23 (m, 2H); 8.91 (d, 1H) ppm.

### EXAMPLE INT24

### N*2*-Ethyl-pyridine-2,5-diamine

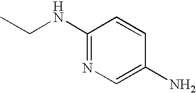

420 mg of the compound that is described under Example INT23) is dissolved in 20 ml of ethanol and mixed with 120 mg of palladium on carbon (10%). It is stirred for 4 hours under hydrogen atmosphere at room temperature. After filtration on diatomaceous earth and after the solvent is condensed in a rotary evaporator, 340 mg of the title compound is obtained.

1H-NMR (DMSO-d6): δ=1.09 (t, 3H); 3.11 (m, 2H); 4.25 (s, 2H); 5.43 (t, 1H); 6.25 (d, 1H); 6.81 (dd, 1H); 7.45 (d, 1H) ppm.

### EXAMPLE INT25

### N-(5-Nitro-pyridin-2-yl)-acetamide

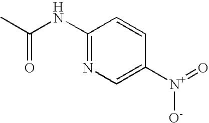

1.12 g of 2-amino-5-nitro-pyridine, 5.1 ml of triethylamine, and a spatula-tip full of DMAP are dissolved in 20 ml of tetrahydrofuran. 0.86 ml of acetyl chloride is added, and it is stirred under reflux for 24 hours. The reaction mixture is mixed with ethyl acetate and washed three times with semi-saturated sodium bicarbonate solution. The organic phase is dried on sodium sulfate. After purification by chromatography on silica gel and after crystallization from ethanol, 340 mg of the title compound is obtained.

1H-NMR (DMSO-d6): δ=2.17 (s, 3H); 8.28 (d, 1H); 8.59 (dd, 1H); 9.16 (d, 1H); 11.25 (s, 1H) ppm.

### EXAMPLE INT26

### N*2*-Ethyl-pyridine-2,5-diamine

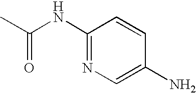

340 mg of the compound that is described under Example INT25) is dissolved in 50 ml of ethanol and 10 ml of dichloromethane and mixed with 120 mg of palladium on carbon (10%). It is stirred for 4 hours under hydrogen atmosphere at room temperature. After filtration on diatomaceous earth and after the solvent is condensed in a rotary evaporator, 275 mg of the title compound is obtained.

1H-NMR (DMSO-d6): δ=2.00 (s, 3H); 5.14 (s, 2H); 6.95 (dd, 1H); 7.66 (d, 1H); 7.73 (d, 1H); 9.99 (s, 1H) ppm.

### EXAMPLE INT27

### Bis-(5-nitro-pyridin-2-yl)-(2-pyrrolidin-1-yl-ethyl)-amine

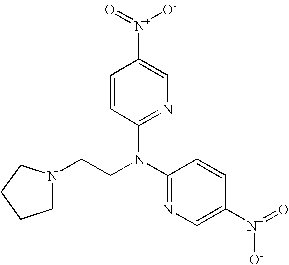

395 mg of 2-chloro-5-nitro-pyridine and 2.70 mg of 2-pyrrolidin-1-yl-ethylamine are dissolved in 5 ml of DMSO and stirred for 6 hours at 100° C. The reaction mixture is mixed with dichloromethane and washed three times with semi-saturated sodium bicarbonate solution. The organic phase is dried on sodium sulfate. After purification by chromatography on silica gel, 51 mg of the title compound is obtained.

1H-NMR (DMSO-d6): δ=1.59 (m, 4H); 2.43 (m, 4H); 2.75 (t, 2H); 4.42 (t, 2H); 7.56 (d, 2H); 8.48 (dd, 2H); 9.19 (d, 2H) ppm.

### EXAMPLE INT28

### Bis-(5-amino-pyridin-2-yl)-(2-pyrrolidin-1-yl-ethyl)-amine

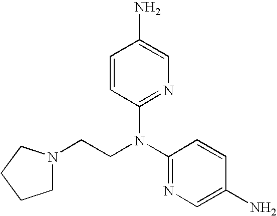

50 mg of the compound that is described under Example INT27) is dissolved in 10 ml of ethanol and mixed with 20 mg of palladium on carbon (10%). It is stirred for 4 hours under hydrogen atmosphere at room temperature. After filtration on diatomaceous earth and after the solvent is condensed in a rotary evaporator, 41 mg of the title compound is obtained.

1H-NMR (DMSO-d6): δ=1.97 (m, 4H); 3.00-3.47 (m,b, 6H); 4.20 (t, 2H); 5.03 (s, 4H); 6.76 (d, 2H); 7.00 (dd, 2H); 7.77 (d, 2H) ppm.

### EXAMPLE INT29

### rac-1,1,1-Trifluoro-2-[4′-nitrophenyl]-propan-2-ol

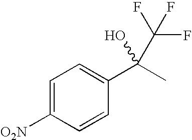

0.7 g of 4-nitroacetophenone is dissolved in 9 ml of THF and mixed with 3.2 ml of (trifluoromethyl)-trimethylsilane and 9 mg of tetra-n-butylammonium fluoride-trihydrate. The solution is stirred for 5 hours at room temperature. For working-up, it is mixed with 16 ml of dilute hydrochloric acid (9+1). After the addition of 200 ml of water, it is extracted with ethyl acetate. The organic phase is washed with concentrated sodium bicarbonate solution and water, dried on magnesium sulfate and concentrated by evaporation. The oil that is obtained is taken up in 40 ml of acetone, mixed with 6.1 ml of hydrochloric acid and stirred for 2 hours at room temperature. It is mixed with sodium bicarbonate solution and extracted with ethyl acetate. The product that is obtained after drying on magnesium sulfate and evaporation of the solvent is purified on silica gel. 0.82 g of the title compound is obtained as almost colorless crystals.

1H-NMR (DMSO-d6): δ=1.74 (s, 3H); 6.99 (s, 1H); 7.88 (d, 2H); 8.26 (d, 2H) ppm.

### EXAMPLE INT30

### 4′-Nitro-N-methyacetanilide

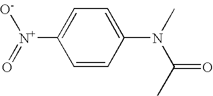

2.5 g of N-(4-nitro-phenyl)-acetamide is dissolved in 50 ml of hot acetone and mixed with 3 g of potassium hydroxide and 3 g of methyl iodide. It is refluxed for 10 minutes. The residue that remains after the evaporation of the acetone is taken up in water. It is extracted with ethyl acetate. The organic phase is washed with saturated sodium chloride solution, dried on magnesium sulfate and concentrated by evaporation. 2.4 g of the title compound is obtained as yellow crystals.

1H-NMR (CDCl3): δ=2.02 (s, 3H); 3.34 (s, 3H); 7.39 (d, 2H); 8.28 (d, 2H) ppm.

### Intermediate Compound INT31

### N-(2-Dimethylamino-ethyl)-3-nitro-benzenesulfonamide

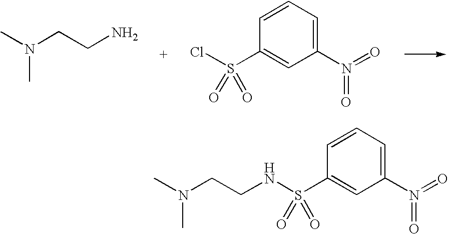

A solution of 3-nitro-benzenesulfonyl chloride (1 equivalent) in acetonitrile is added in drops at 0° C. to a solution of N*1*,N*1*-dimethyl-ethane-1,2-diamine (2.2 equivalents) in acetonitrile and stirred overnight at room temperature. The reaction is completed by adding sodium hydroxide solution (1N), and it is extracted three times with 2-methoxy-2-methyl-propane. Solvent is removed from the combined organic phases in a rotary evaporator, and purified by column chromatography. The title compound is obtained with a yield of 43%.

1H-NMR (CDCl3, 300 MHz), (selected peaks) δ=2.11 (s, 6H); 2.39 (m, 2H); 3.03 (m, 2H); 7.75 (t, 1H); 8.21 (dd, 1H); 8.42 (dd, 1H); 8.72 (m, 1H).

### Intermediate Compound INT32

### Dimethyl-[2-(4-nitro-phenoxy)-ethyl]-amine

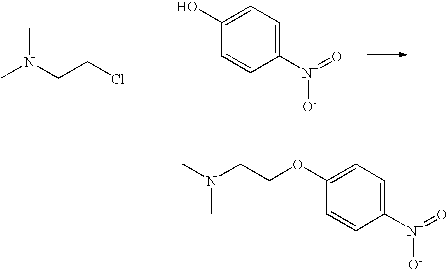

A suspension of 10 g of 4-nitrophenol, 11 g of (2-chloro-ethyl)-dimethyl-amine and 27.1 g of potassium carbonate in 200 ml of acetone is refluxed for 15 hours. Solvent is removed from the batch in a vacuum, and the residue is taken up in ethyl acetate. It is extracted three times with 200 ml each of sodium hydroxide solution (1N), and the combined organic phases are dried on sodium carbonate, the solvent is distilled off in a rotary evaporator, and the title compound is obtained with a yield of 50%.

1H-NMR (CDCl3, 300 MHz), (selected peaks) δ=2.35 (s, 6H); 2.78 (m, 2H); 4.16 (m, 2H); 6.97 (d, 2H); 8.19 (d, 2H).

### Intermediate Compound INT33

### 4-(2-Dimethylamino-ethoxy)-phenylamine

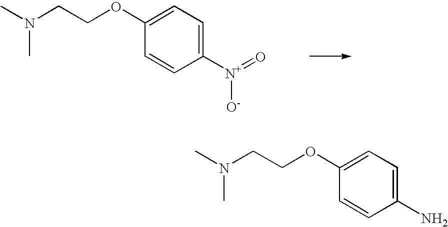

14.9 g of the compound that is produced under Example INT LW32) is dissolved in 300 ml of methanol and mixed with 2 g of palladium on carbon (10%), and it is stirred under hydrogen atmosphere at room temperature. After hydrogen absorption is completed, catalyst is filtered out, and solvent is removed from the crude product in a rotary evaporator. The title compound is obtained in a quantitative yield. The crude product is used without further purification in the next stage.

1H-NMR (CDCl3, 300 MHz), (selected peaks) δ=2.35 (s, 6H); 2.70 (m, 2H); 4.00 (m, 2H); 6.63 (d, 2H); 6.79 (d, 2H).

The following intermediate compounds are produced analogously to the above-described processes.

| | | | | Synthesis |
|---|---|---|---|---|
| Example | | Molecular | MS (ESI) | as in the |
| No. | Structure | Weight | [M + 1]⁺ | Case of |
| INT34 | 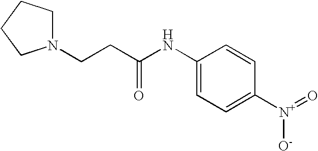 | 263.299 | 264 | INT13 |
| INT35 | 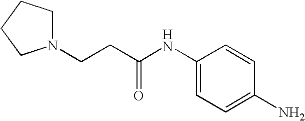 | 233.32 | 234 | INT14 |
| INT36 | 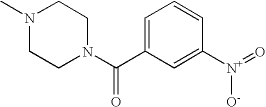 | 249.272 | 250 | INT18 |
| INT37 | 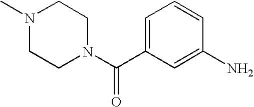 | 219.289 | 220 | INT19 |
| INT38 | 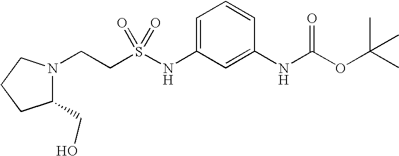 | 399.513 | 400 | INT10 |
| INT39 | 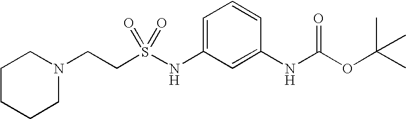 | 383.514 | 384 | INT10 |
| INT40 | 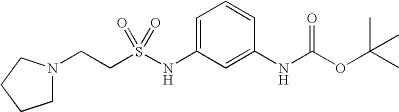 | 369.486 | 370 | INT10 |
| INT41 | 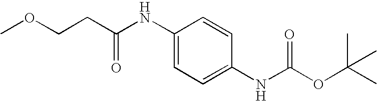 | 294.354 | 295 | INT11 |
| INT42 | 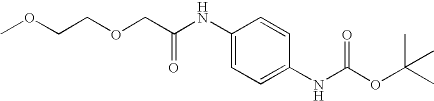 | 324.38 | 325 | INT11 |
| INT43 | 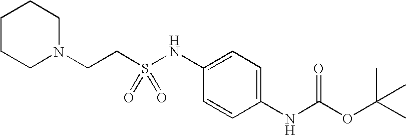 | 383.514 | 384 | INT10 |
| INT44 | 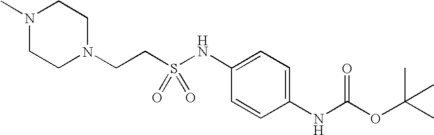 | 398.528 | 399 | INT10 |
| INT45 | 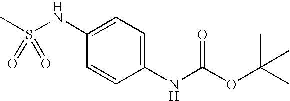 | 286.352 | 287 | INT9 |
| INT46 | 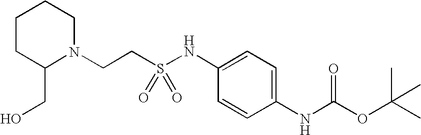 | 413.54 | 414 | INT10 |
| INT47 | 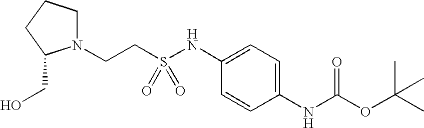 | 399.513 | 400 | INT10 |
| INT48 | 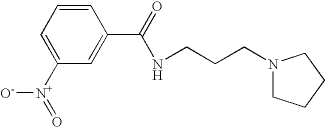 | 277.33 | 278 | INT20 |
| INT49 | 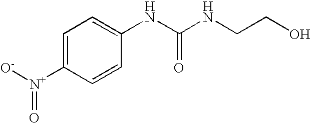 | 225.203 | 226 | INT20 |
| INT50 | 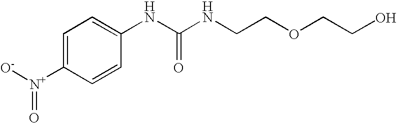 | 269.255 | 270 | INT20 |
| INT51 | 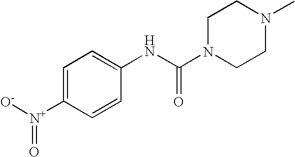 | 264.283 | 265 | INT20 |
| INT52 | 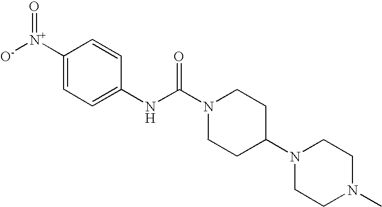 | 347.417 | 348 | INT20 |
| INT53 | 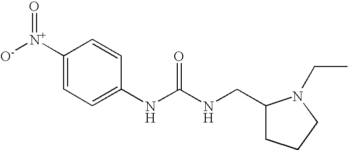 | 292.337 | 293 | INT20 |
| INT54 | 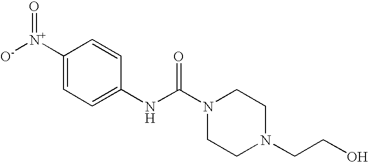 | 294.309 | 295 | INT20 |
| INT55 | 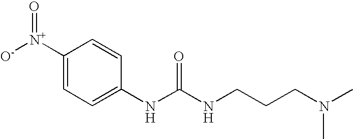 | 266.299 | 267 | INT20 |
| INT56 | 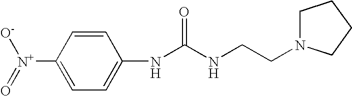 | 278.31 | 279 | INT20 |
| INT57 | 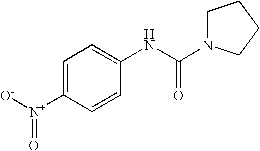 | 235.24 | 236 | INT20 |
| INT58 | 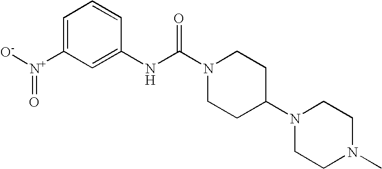 | 347.42 | 348 | INT20 |
| INT59 | 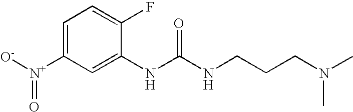 | 284.49 | 285 | INT20 |
| INT60 | 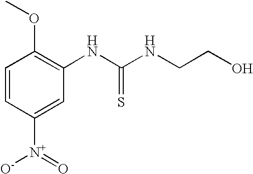 | 271.30 | 272 | INT20 |
| INT61 | 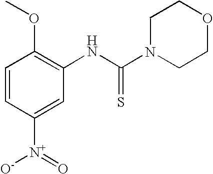 | 297.34 | 298 | INT20 |
| INT62 | 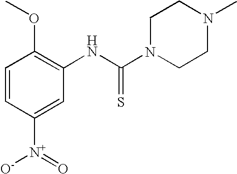 | 310.38 | 311 | INT20 |
| INT63 | 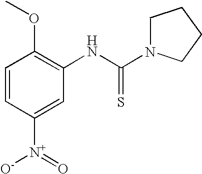 | 281.34 | 282 | INT20 |
| INT64 | 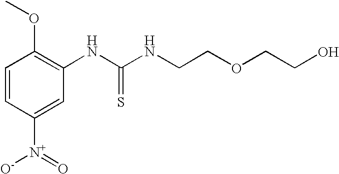 | 315.35 | 316 | INT20 |
| INT65 | 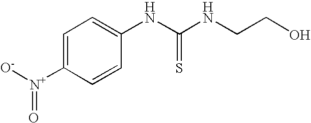 | 241.47 | 242 | INT20 |
| INT66 | 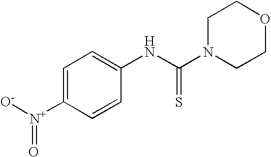 | 267.31 | 268 | INT20 |
| INT67 | 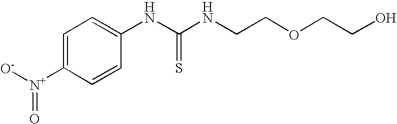 | 285.32 | 286 | INT20 |
| INT68 | 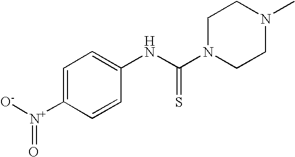 | 280.37 | 281 | INT20 |
| INT69 | 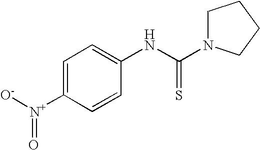 | 251.31 | 252 | INT20 |
| INT70 | 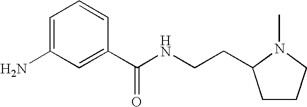 | 247.34 | 248 | INT21 |
| INT71 | 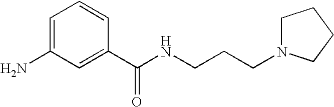 | 247.34 | 248 | INT21 |
| INT72 | 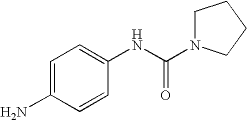 | 205.26 | 206 | INT21 |
| INT73 | 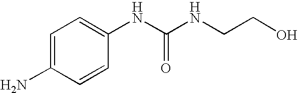 | 195.221 | 196 | INT21 |
| INT74 | 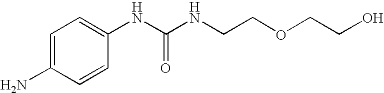 | 239.273 | 240 | INT21 |
| INT75 | 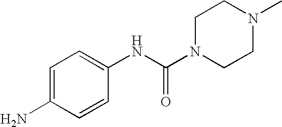 | 234.301 | 235 | INT21 |
| INT76 | 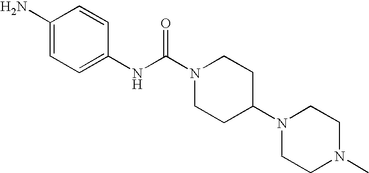 | 317.434 | 318 | INT21 |
| INT77 | 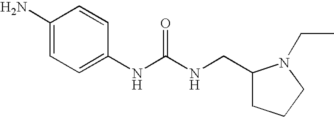 | 262.355 | 263 | INT21 |
| INT78 | 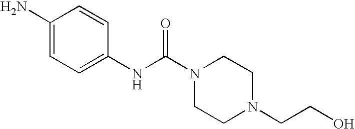 | 264.327 | 265 | INT21 |
| INT79 | 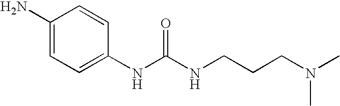 | 236.317 | 237 | INT21 |
| INT80 | 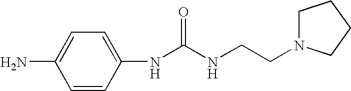 | 248.328 | 249 | INT21 |
| INT81 | 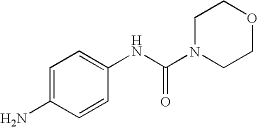 | 221.26 | 222 | INT21 |
| INT82 | 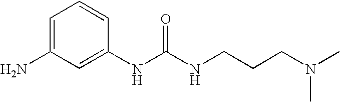 | 236.317 | 237 | INT21 |
| INT83 | 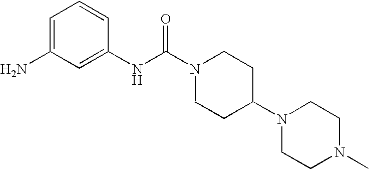 | 317.434 | 318 | INT21 |
| INT84 | 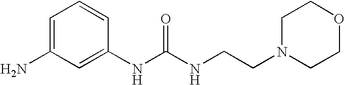 | 264.33 | 265 | INT21 |
| INT85 | 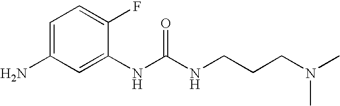 | 254.307 | 255 | INT21 |
| INT86 | 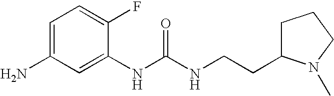 | 280.345 | 281 | INT21 |
| INT87 | 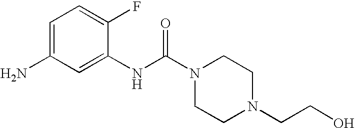 | 282.317 | 283 | INT21 |
| INT88 | 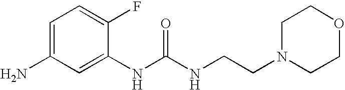 | 282.32 | 283 | INT21 |
| INT89 | 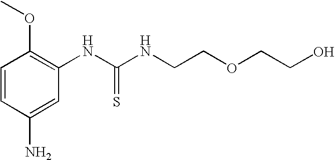 | 285.37 | 286 | INT21 |
| INT90 | 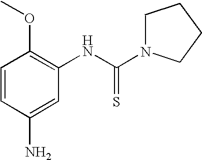 | 251.35 | 252 | INT21 |
| INT91 | 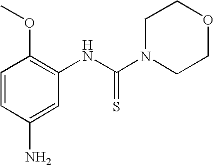 | 267.35 | 268 | INT21 |
| INT92 | 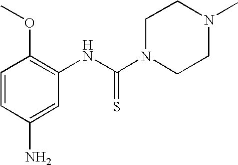 | 280.39 | 281 | INT21 |
| INT93 | 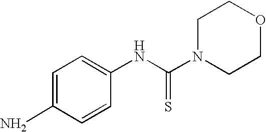 | 237.33 | 238 | INT21 |
| INT94 | 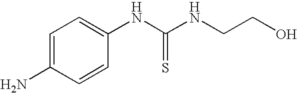 | 211.29 | 212 | INT21 |
| INT95 | 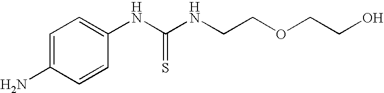 | 255.34 | 256 | INT21 |
| INT96 | 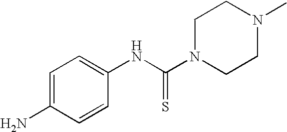 | 250.37 | 251 | INT21 |
| INT97 | 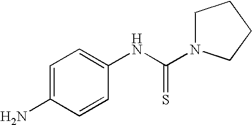 | 221.33 | 222 | INT21 |
| INT98 | 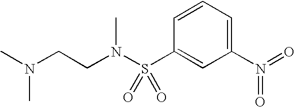 | 287.34 | 288 | INT31 |
| INT99 | 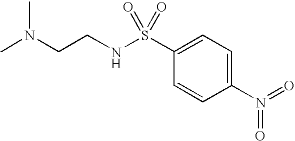 | 273.31 | 274 | INT31 |
| INT100 | 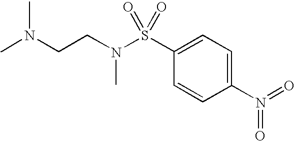 | 287.34 | 288 | INT31 |
| INT101 | 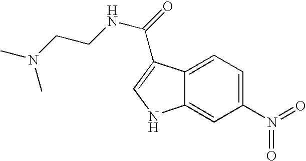 | | 277 | 167 |
| INT102 | 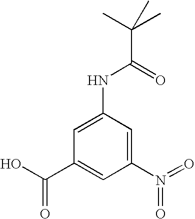 | | 267 | 167 |
| INT103 | 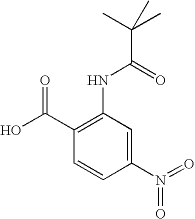 | | 267 | 167 |
| INT104 | 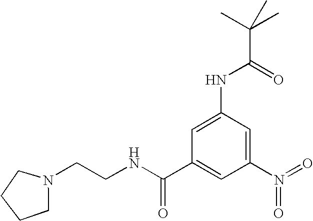 | | 363 | 167 |
| INT105 | 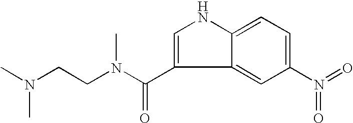 | | 291 | 167 |
| INT106 | 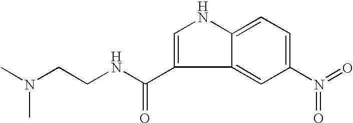 | | 277 | 167 |
| INT107 | 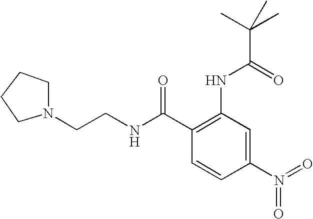 | | 363 | 167 |
| INT108 | 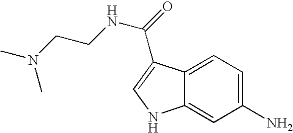 | | 247 | INT8 |
| INT109 | 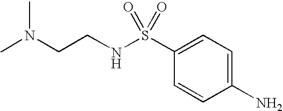 | | 244 | INT8 |
| INT110 | 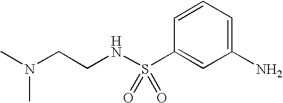 | | 244 | INT8 |
| INT111 | 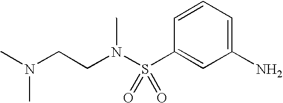 | | 258 | INT8 |
| INT112 | 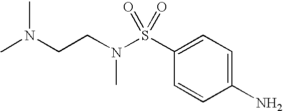 | | 258 | INT8 |
| INT113 | 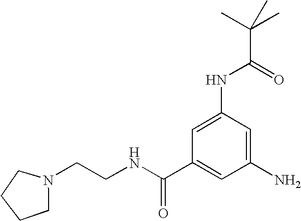 | | 333 | INT8 |
| INT114 | 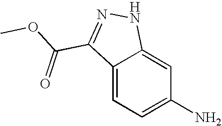 | | 192 | INT8 |
| INT115 | 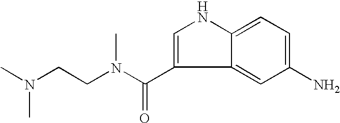 | | 261 | INT8 |
| INT116 | 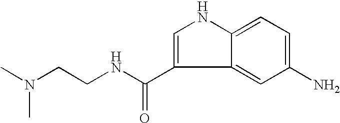 | | 247 | INT8 |
| INT117 | 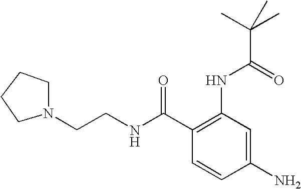 | | 333 | INT8 |
| INT118 | 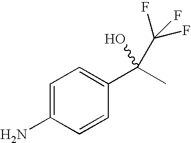 | 205.181 | 206 | INT4 |
| INT119 | 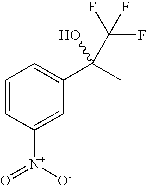 | 235.164 | 236 | INT29 |
| INT120 | 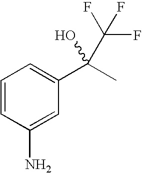 | 205.181 | 206 | INT4 |
| INT121 | 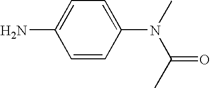 | 164.208 | 165 | INT4 |

The following intermediate compounds are already disclosed in Patent Application PCT/EP03/04450 and are not claimed in this application.

### EXAMPLE INT122)

### Cyano-ethylthiocarbamoyl-acetic acid ethyl ester

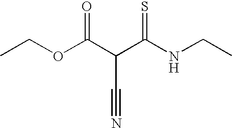

4.25 ml of ethyl isothiocyanate is added at 25° C. to a mixture that consists of 5 g of cyanoacetic acid ethyl ester and 5 ml of triethylamine. Then, it is allowed to stir for 6 more hours at 50° C. Then, the reaction mixture is concentrated by evaporation in a vacuum. The residue is taken up in ethanol and poured onto 150 ml of ice-cold 1N hydrochloric acid. It is allowed to stir for 3 more hours at 25° C., and then the residue is filtered off. The solid that is obtained is rewashed with water. 7 g of product is obtained.

Molar mass=200.261; MS (ESI): [M+1]+=201.

### EXAMPLE INT123)

### (E or Z)-Cyano-(3-ethyl-4-oxo-thiazolidin-2-ylidene)-acetic acid ethyl ester

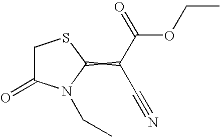

7.82 g of the compound that is described under Example INT122) is dissolved in 100 ml of tetrahydrofuran. A solution of 3.9 ml of bromoacetyl chloride is slowly added and allowed to stir for 8 more hours at 25° C. Then, the reaction mixture is poured onto saturated aqueous sodium bicarbonate solution. It is allowed to stir for 1 more hour and then extracted with ethyl acetate. The organic phase is washed with saturated sodium chloride solution, dried on sodium sulfate and concentrated by evaporation in a vacuum. The crude product that is obtained is recrystallized from a mixture of ethyl acetate/diisopropyl ester. 7.7 g of product is obtained.

¹H-NMR (CDCl₃): δ=1.36 (6H); 3.70 (2H); 4.32 (4H) ppm.

### EXAMPLE INT124)

### (E or Z)-Cyano-(5-(E/Z)-ethoxymethylene-3-ethyl-4-oxo-thiazolidin-2-ylidene)-acetic acid ethyl ester

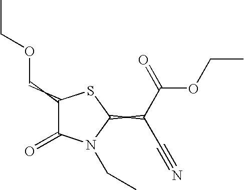

A mixture that consists of 1.54 g of the substance that is described under Example INT123), 2.5 ml of triethyl orthoformate and 3.5 ml of acetic acid anhydride are refluxed for 8 hours. Then, the reaction mixture is poured onto ice water. It is allowed to stir for 3 more hours, and then the residue is filtered off. The solid that is obtained is rewashed with water. 1.28 g of product is obtained.

¹H-NMR (CDCl₃): δ=1.38 (9H); 4.20-4.40 (6H); 7.72 (1H) ppm.

### EXAMPLE INT125)

### (E or Z)-Cyano-(3-ethyl-4-oxo-thiazolidin-2-ylidene)-acetic acid allyl ester

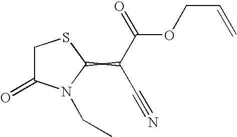

A solution of 37.6 ml of cyanoacetic acid allyl ester in 60 ml of dimethylformamide is added to a suspension of 12.8 g of sodium hydride (60%) in 200 ml of dimethylformamide at 0° C. It is stirred for 10 more minutes at 0° C., and then a solution of 28.0 ml of ethyl isothiocyanate in 60 ml of dimethylformamide is added. Then, it stirred for 2 more hours at 25° C. Then, a solution of 32 ml of bromoacetyl chloride in 60 ml of dimethylformamide is added at 0° C., and it is stirred for 15 more hours at 25° C. Then, the reaction mixture is poured onto saturated sodium bicarbonate solution. It is extracted with ethyl acetate, the organic phase is washed with saturated sodium chloride solution, dried on sodium sulfate and concentrated by evaporation in a vacuum. The crude product is purified by column chromatography on silica gel with a mixture that consists of hexane/ethyl acetate. 33.9 g of product is obtained.

1H-NMR (CDCl₃): δ=1.23(3H); 4.11 (2H); 4.71 (2H); 5.25 (1H); 5.37 (1H); 5.90-6.04 (1H) ppm.

### EXAMPLE INT126)

### (E or Z)-Cyano-(5-(E/Z)-ethoxymethylene-3-ethyl-4-oxo-thiazolidin-2-ylidene)-acetic acid allyl ester

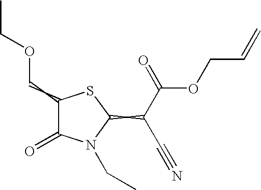

Analogously to Example INT124), 14.8 g of product is obtained from 12.8 g of the compound that is described under Example INT125), 20.9 ml of triethyl orthoformate and 29.4 ml of acetic acid anhydride.

1H-NMR (CDCl₃): δ=1.32-1.45 (6H); 4.23 (2H); 4.38 (2H); 4.73 (2H); 5.29 (1H); 5.41 (1H), 5.92-6.05 (1H); 7.72 (1H) ppm.

### EXAMPLE INT127)

### (E or Z)-Cyano-(3-ethyl-4-oxo-thiazolidin-2-ylidene)-acetic acid benzyl ester

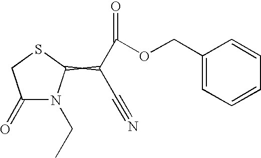

A solution of 1.75 g of cyanoacetic acid benzyl ester in 10 ml of dimethylformamide is added to a suspension of 0.4 g of sodium hydride (60%) in 5 ml of dimethylformamide at 0° C. It is stirred for 10 more minutes at 0° C., and then a solution of 876 μl of ethyl isothiocyanate in 5 ml of dimethylformamide is added. Then, it is stirred for 2 more hours at 25° C. Then, a solution of 1 ml of bromoacetyl chloride in 5 ml of dimethylformamide is added at 0° C., and it is stirred for 15 more hours at 25° C. Then, the reaction mixture is poured onto saturated sodium bicarbonate solution. It is extracted with dichloromethane, the organic phase is washed with saturated sodium chloride solution, dried on sodium sulfate and concentrated by evaporation in a vacuum. The crude product is purified by column chromatography on silica gel with a mixture that consists of hexane/ethyl acetate. 1.1 g of product is obtained.

1H-NMR (CDCl₃): δ=1.35 (3H); 3.70 (2H); 4.30 (2H); 5.31 (2H), 7.30-7.48 (5H) ppm.

### EXAMPLE INT128)

### (E or Z)-Cyano-(5-(E/Z)-ethoxymethylene-3-ethyl-4-oxo-thiazolidin-2-ylidene)-acetic acid benzyl ester

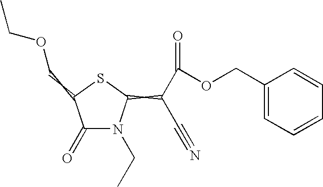

Analogously to Example INT124), 1.26 g of product is obtained from 11 g of the compound that is described under Example INT127), 1.49 ml of triethyl orthoformate and 2.1 ml of acetic acid anhydride.

1H-NMR (CDCl₃): δ=1.30-1.45 (6H); 4.25 (2H); 4.38 (2H); 5.29 (2H); 7.30-7.48 (5H), 7.72 (1H) ppm.

### EXAMPLE INT129)

### [3-Butyl-4-oxo-thiazolidin-(2-(E or Z))-ylidene]-cyano-acetic acid ethyl ester

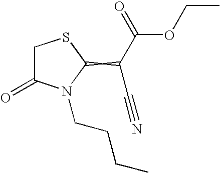

Analogously to the above-described process, the production can be obtained.

1H-NMR (DMSO-d6): δ=0.90 (t, 3H); 1.25 (t, 3H); 1.32 (m, 2H); 1.59 (m, 2H); 3.97 (s, 2H); 4.15 (t, 2H); 4.22 (q, 2H) ppm.

### EXAMPLE INT130)

### [3-Butyl-5-[1-ethoxy-meth-(E/Z)-ylidene]-4-oxo-thiazolidin-(2-(E or Z))-ylidene]-cyano-acetic acid ethyl ester

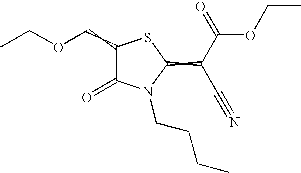

Analogously to Example INT124), the product can be obtained from Example INT129).

1H-NMR (DMSO-d6): δ=0.90 (t, 3H); 1.20-1.40 (m, 8H); 1.61 (m, 2H); 4.15 (t, 2H); 4.23 (q, 2H); 4.39 (q, 2H) ppm.

### EXAMPLE INT131

### (E or Z)-Cyano-(3-ethyl-4-oxo-5-(E/Z)-{[4-(2-pyrrolidin-1-yl-ethyl)-phenylamino]-methylene}-thiazolidin-2-ylidene)-acetic acid ethyl ester

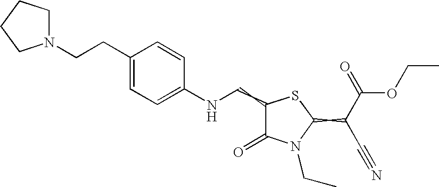

3.43 g of the compound that is described under Example INT4) is dissolved in 60 ml of ethanol. 4.11 g of the compound that is described under Example INT124) is added, and it is stirred under reflux for 15 hours. After cooling, the reaction mixture is filtered, and the solid is recrystallized from ethanol. 4.95 g of the title compound is obtained as a pH-dependent 5-(E/Z)-isomer mixture.

1H-NMR (DMSO-d6, stored with K2CO3, main isomer): δ=1.16-1.33 (m, 6H); 1.59-1.75 (m, 4H); 2.38-2.50 (m, 4H); 2.59 (t, 2H); 2.69 (t, 2H); 4.13-4.31 (m, 4H); 7.10-7.29 (m, 4H); 8.19 (s, 1H); 10.53 (s, 1H) ppm.

### EXAMPLE INT132

### (E or Z)-Cyano-(3-ethyl-4-oxo-5-(E/Z)-{[3-(3-pyrrolidin-1-yl-propionylamino)-phenylamino]-methylene}-thiazolidin-2-ylidene)-acetic acid ethyl ester

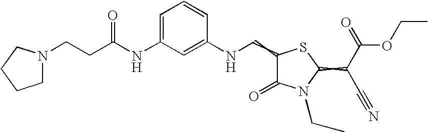

3.0 g of the compound that is described under Example INT17) is dissolved in 50 ml of ethanol. 3.82 g of the compound that is described under Example INT124) is added and stirred under reflux for 4 hours. The solvent is condensed in a rotary evaporator. After purification by chromatography on silica gel, 5.3 g of the title compound is obtained as a pH-dependent 5-(E/Z)-isomer mixture.

1H-NMR (DMSO-d6, stored with K2CO3, main isomer): δ=1.18-1.34 (m, 6H); 1.62-1.78 (m, 4H); 2.48-2.62 (m, 6H); 2.78 (t, 2H); 4.16-4.32 (m, 4H); 6.99 (d, 1H); 7.18 (d, 1H); 7.29 (t, 1H); 7.75 (s, 1H); 8.10 (s, 1H); 10.19 (s, 1H); 10.60 (s, 1H) ppm.

The following compounds were produced analogously to the above-described process.

| | | | Molecu- | |
|---|---|---|---|---|
| | | | lar | Educt/ |
| | | | Weight/ | Syn- |
| | | | MS | thesis as |
| Example | | | (ESI) | in the |
| No. | Structure and Name | ¹H—NMR | [M + 1]⁺ | Case of |
| INT133 | 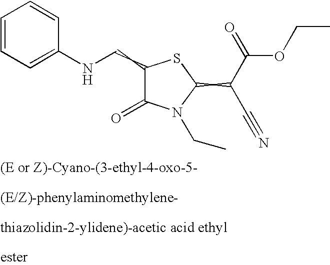 | 1.18-1.31 (m, 6H); 4.15-4.31 (m, 4H); 7.10 (m, 1H); 7.28-7.41 (m, 4H); 8.20 (d, 1H); 10.52 (d, 1H) ppm. | MW: 343.41 MS (ESI) [M + 1]⁺: 344 | INT124/ INT132 |
| INT134 | 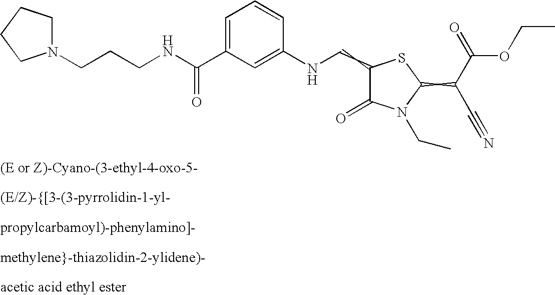 | 1.15-1.32 (m, 6H); 1.61-1.75 (m, 6H); 2.38-2.49 (m, 6H); 3.18-3.33 (m, 2H); 4.18 (q, 2H); 4.23 (q, 2H); 7.29 (d, 1H); 7.38 (t, 1H); 7.48 (d, 1H); 7.61 (s, 1H); 8.36 (s, 1H); 8.58 (t, lH); 10.61 (s, 1H) ppm. | MW: 497.62 MS (ESI) [M + 1]⁺: 498 | INT124/ INT132 |
| INT135 | 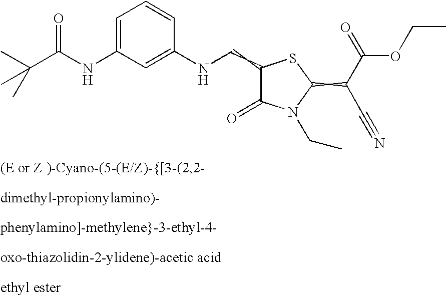 | 1.16-1.33 (m, 15H); 4.17-4.32(m, 4H); 6.97 (d, 1H); 7.27 (t, 1H); 7.38 (d, 1H); 7.75 (s, 1H); 8.13 (s, 1 H); 9.26 (s, 1H); 10.65 (s, 1H) ppm. | MW: 442.54 MS (ESI) [M + 1]⁺: 443 | INT124/ INT132 |
| INT136 | 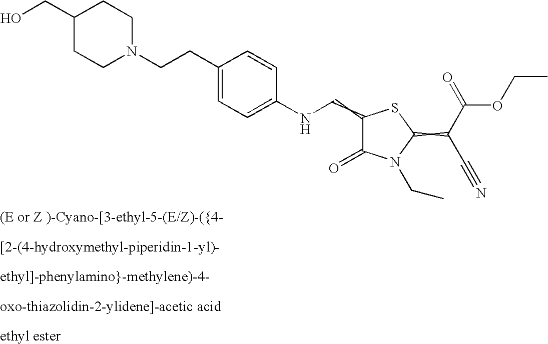 | 1.00-1.38 (m, 9H); 1.63 (d, 2H); 1.90 (t, 2H); 2.39-2.48 (m, 2H); 2.62-2.75 (m, 2H); 2.85-2.98 (m, 2H); 3.23 (t, 2H); 4.15-4.30 (m, 4H); 4.40 (t, 1H); 7.12-7.29 (m, 4H); 8.18 (s, 1H); 10.50 (s, 1H) ppm. | MW: 484.62 MS (ESI) [M + 1]⁺: 485 | INT124/ INT132 |
| INT137 | 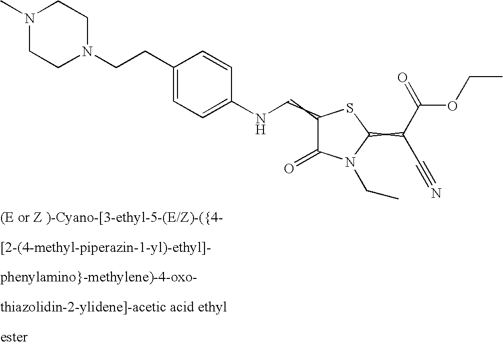 (E or Z)-Cyano-[3-ethyl-5-(E/Z)-({4- [2-(4-methyl-piperazin-1-yl)-ethyl]phenylamino}-methylene)-4-oxo- thiazolidin-2-ylidene]-acetic acid ethyl ester | 1.17-1.31(m,6H); 2.15 (s, 3H); 2.20-2.49 (m, 10H); 2.68 (t, 2H); 4.16-4.32 (m, 4H); 7.15-7.31 (m,4H); 8.18 (s, 1H); 10.50 (s, 1H) ppm. | MW: 469.61 MS (ESI) [M + 1]⁺: 470 | INT124/ INT132 |
| INT138 | 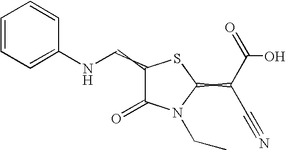 | | MW: 315.35 MS (ESI) [M + 1]⁺: 316 | INT133/ 142 |

The following examples describe the production of compounds according to the invention without the latter being limited to these examples. These compounds can also be used as intermediate substances in the production of substances of general formula (I) according to the invention. In this connection, the ester is cleaved into the free acids. Noteworthy is the fact that the compounds that have an allyl ester can be better cleaved into the free acid than ethyl ester.

### EXAMPLE 1

### (E or Z)-Cyano-(3-ethyl-5-(E/Z)-{[4-(2-morpholin-4-yl-ethanesulfonylamino)-phenylamino]-methylene}-4-oxo-thiazolidin-2-ylidene)-acetic acid ethyl ester

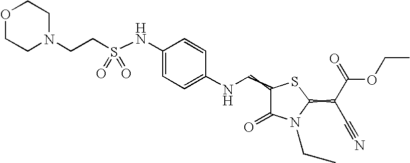

58 mg of the compound that is described under Example INT10) is dissolved in 2 ml of dichloromethane, mixed with 5 ml of trifluoroacetic acid, and stirred for 30 minutes at room temperature. The reaction mixture is concentrated by evaporation in a rotary evaporator. The residue is dissolved in 3 ml of ethanol. 0.7 ml of triethylamine and 36 mg of the compound that is described under Example INT124) are added and stirred under reflux for 3 hours. The solvent is condensed in a rotary evaporator. After purification by chromatography on silica gel, 55 mg of the title compound is obtained as a pH-dependent 5-(E/Z)-isomer mixture.

1H-NMR (DMSO-d6, stored with K2CO3, main isomer): δ=1.15-1.31 (m, 6H); 2.30 (m, 4H); 2.66 (t, 2H); 3.22 (t, 2H); 3.50 (m, 4H); 4.14-4.31 (m, 4H); 7.19 (d, 2H); 7.29 (d, 2H); 8.18 (s, 1H); 9.50-10.75 (b, 2H) ppm.

### EXAMPLE 2

### (E or Z)-Cyano-[3-ethyl-4-oxo-5-(E/Z)-({4-[(pyrrolidine-1-carbonyl)-amino]-phenylamino}-methylene)-thiazolidin-2-ylidene]-acetic acid ethyl ester

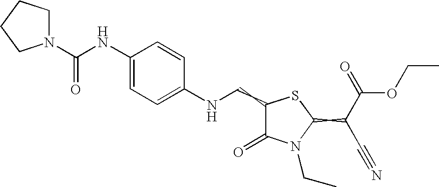

205 mg of the compound that is described under Example INT21) is dissolved in 10 ml of ethanol. 100 mg of the compound that is described under Example INT124) is added, and it is stirred under reflux for 15 hours. After cooling, the reaction mixture is filtered, and the solid is recrystallized from ethanol. 118 mg of the title compound is obtained as a pH-dependent 5-(E/Z)-isomer mixture.

1H-NMR (DMSO-d6, stored with K2CO3, main isomer): δ=1.21 (m, 6H), 1.81 (m, 4H), 3.32 (m, 4H), 4.20 (m, 2H), 7.18 (d, 2H), 7.50 (d, 2H), 8.12 (s, 1H) ppm.

### EXAMPLE 3

### (E or Z)-Cyano-(3-ethyl-5-(E/Z-({4-[3-(4-methyl-piperazin-1-yl)-propionylamino]-phenylamino}-methylene}-4-oxo-thiazolidin-2-ylidene)-acetic acid allyl ester

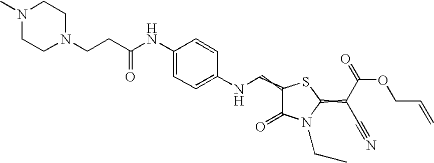

1 g of the compound that is described under Example INT126) and 0.93 g of the compound that is described under Example INT14) are stirred in 20 ml of ethanol for 15 hours at 100° C. The reaction mixture is evaporated to the dry state in a rotary evaporator. The thus obtained crude product is purified by chromatography on silica gel. 1.6 g of the title compound is obtained as a pH-dependent 5-(E/Z)-isomer mixture.

1H-NMR (DMSO-d6, main isomer): δ=1.25 (3H); 2.12 (3H); 2.21-2.55 (10H) 2.60 (2H); 4.23 (2H); 4.70 (2H); 5.25 (1H); 5.88 (1H); 5.90-6.06 (1H); 7.27 (2H); 7.55 (2H); 8.16 (1H); ppm.

### EXAMPLE 4

### (E or Z)-Cyano-(3-ethyl-5-(E/Z-({4-[3-(4-methyl-piperazin-1-yl)-propionylamino]-phenylamino}-methylene}-4-oxo-thiazolidin-2-ylidene)-acetic acid benzyl ester

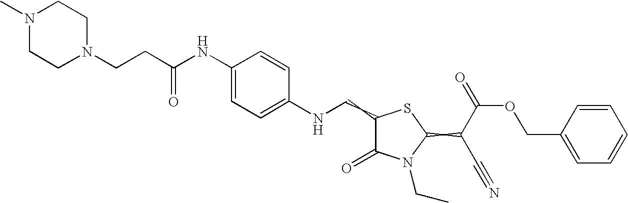

Analogously to Example 3), 7.4 g of the title compound is obtained by reaction of 5 g of the compound in 100 ml of ethanol that is described in Example INT128) and 4 g of the compound in 100 ml of ethanol that is described in Example INT14).

1H-NMR (DMSO-d6, main isomer): δ=1.23 (3H); 2.16 (3H); 2.22-2.57 (10H); 2.61 (2H); 4.23 (2H); 5.28 (2H); 7.26 (2H); 7.31-7.48 (5H); 7.58 (2H); 8.16 (1H); ppm.

### EXAMPLE 5

### (E or Z)-Cyano-(3-ethyl-5-(E/Z)-{[4-(2-pyrrolidin-1-yl-ethylcarbamoyl)-phenylamino]-methylene}-4-oxo-thiazolidin-2-ylidene)-acetic acid allyl ester

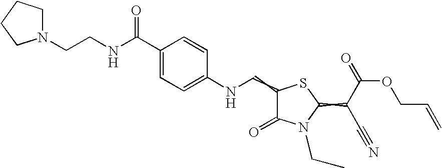

12.2 g of the compound that is described under Example 50), 5.5 ml of triethylamine and 12.8 g of TBTU are introduced into 150 ml of DMF and stirred for 30 minutes at room temperature. 4.5 g of N-(2-aminoethyl)-pyrrolidine is added, and it is stirred overnight at room temperature. The reaction mixture is mixed with sodium chloride solution and extracted with a dichloromethane/methanol mixture. After purification by chromatography on silica gel, 13.2 g of the title compound is obtained as a pH-dependent 5-(E/Z)-isomer mixture.

1H-NMR (DMSO-d6, main isomer): δ=1.23 (3H); 1.75-2.33 (4H); 2.90-3.13 (4H); 3.52 (2H); 4.23 (2H); 4.72 (2H); 5.26 (1H). 5.89 (1H); 5.91-6.07 (1H); 7.40 (2H); 7.90 (2H); 8.25 (1H); 8.69 (1H); ppm.

The following compounds are produced analogously to the above-described process.

| | | | Molecu- | |
|---|---|---|---|---|
| | | | lar | Educt/ |
| | | | Weight/ | Syn- |
| | | | MS | thesis as |
| Exam- | | | (ESI) | in the |
| ple No. | Structure and Name | ¹H—NMR | [M + 1]⁺ | Case of |
| 6 | 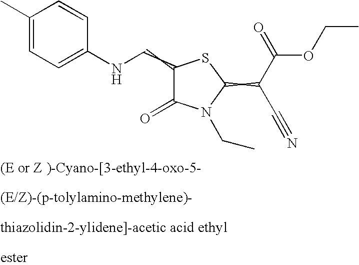 | 1.16-1.32 (m, 6H); 2.27 (s, 3H); 4.15-4.31 (m, 4H); 7.17 (d, 2H); 7.21 (d, 2H); 8.16 (s, 1H); 10.50(s, 1H) ppm. | MW: 357.43 MS (ESI) [M + 1]⁺: 358 | INT124/ INT131 |
| 7 | 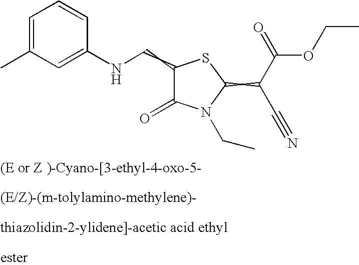 | 1.20-1.31 (m, 6H); 2.30 (s, 3H); 4.20-4.29 (m, 4H); 6.92 (d, 1H); 7.10 (d, 1H); 7.16 (s, 1H); 7.25 (t, 1H); 8.20 (s, 1H); 10.50 (s, 1H) ppm. | MW: 357.43 MS (ESI) [M + 1]⁺: 358 | INT124/ INT131 |
| 8 | 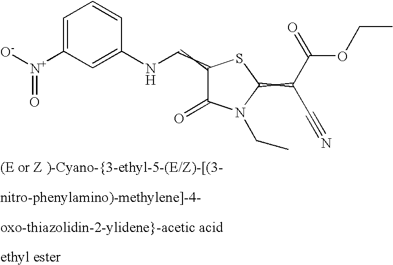 | 1.18-1.32(m, 6H); 4.17-4.31 (m, 4H); 7.61 (t, 1H); 7.81 (d, 1H); 7.88 (d, 1H); 8.13 (s, 1H); 8.32 (s, 1H); 10.70 (s, 1H) ppm. | MW: 388.40 MS (ESI) [M + 1]⁺: 389 | INT124/ INT131 |
| 9 | 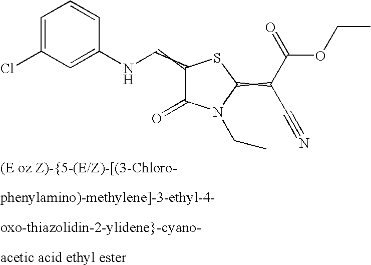 | 1.16-1.30 (m, 6H); 4.18 (q, 2H); 4.23 (q, 2H); 7.00 (d, 1H); 7.08 (d, 1H); 7.12 (s, 1H); 7.28 (t, 1H); 8.28 (s, 1H); 10.51 (s, 1H) ppm. | MW: 377.85 MS (ESI) [M + 1]⁺: | INT124/ INT131 |
| 10 | 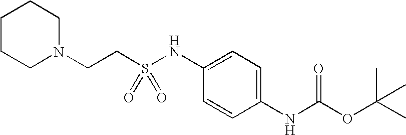 | 1.16-1.30 (m, 6H); 1.35 (t, 3H); 4.17-4.30 (m, 4H); 4.35 (q, 2H); 7.12 (s, 1H); 7.28 (d, 1H); 7.45 (d, 1H); 7.58 (s, 1H); 8.20 (s, 1H); 10.61 (s, 1H); 11.93 (s, 1H) ppm. | MW: 454.51 MS (ESI) [M + 1]⁺: 455 | INT124/ INT131 |
| 11 | 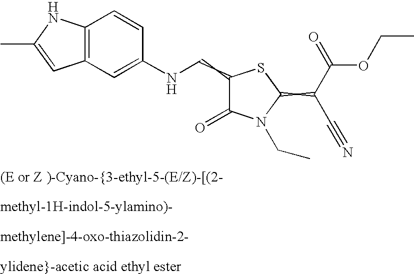 | 1.13-1.34 (m, 6H); 2.38 (s, 3H); 4.12-4.32 (m, 4H); 6.12 (s, 1H); 6.96 (d, 1H); 7.25 (d, 1H); 7.33 (s, 1H); 8.15 (s, 1H); 10.56 (s, 1H); 11.98 (s, 1H) ppm. | MW: 396.47 MS (ESI) [M + 1]⁺: 397 | INT124/ INT131 |
| 12 | 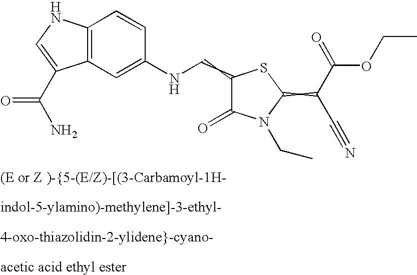 | 1.16-1.34 (m, 6H); 4.15-4.32 (m, 4H); 6.89 (s, 1H); 7.18 (d, 1H); 7.35-7.52 (m, 2H); 8.00-8.10 (m, 2H); 8.20 (d, 1H); 10.75 (d, 1H); 11.60 (s, 1H) ppm. | MW: 425.47 MS (ESI) [M + 1]⁺: 426 | INT124/ INT131 |
| 13 | 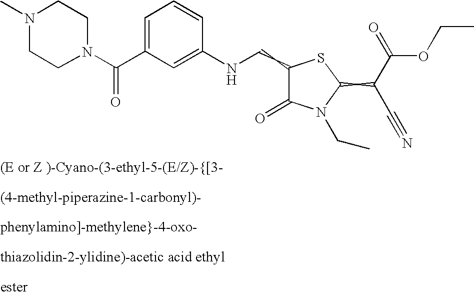 | 1.17-1.34 (m, 6H); 2.20 (s, 3H); 2.23-2.42 (m, 4H); 3.61 (s, 1H); 4.15-4.32 (m, 2H); 7.01-7.10 (m, 1 H); 7.31 (s, 1H); 7.47-7.36 (m, 2H); 8.25 (s, 1H); 10.57 (s, 1H) ppm. | MW: 469.56 MS (ESI) [M + 1]⁺: 470 | INT124/ 1 |
| 14 | 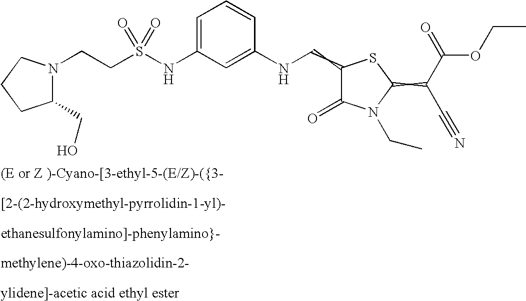 | 1.14-1.32 (m, 6H); 1.44-1.90 (m, b, 5H); 2.50-3.50 (m, b, 9H); 4.12-4.31 (m, 4H); 6.91 (d, 1H); 7.09 (d, 1H); 7.18 (s, 1H); 7.31 (t, 1 H); 8.12 (d, 1H); 9.91 (s, 1H); 10.62 (d, 1H) ppm. | MW: 549.67 MS (ESI) [M + 1]⁺: 550 | INT124/ 1 |
| 15 | 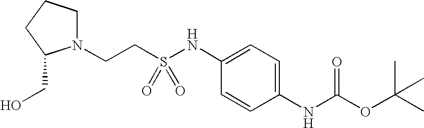 | 1.15-1.53 (m, 12H); 2.25-2.50 (m, 6H); 2.68-2.85 (m, 2H); 4.18-4.31 (m, 4H); 6.92 (d, 1H); 7.08 (d, 1H); 7.17 (s, 1H); 7.31 (t, 1H); 8.12 (d, 1H); 10.01 (s, 1H); 10.62 (d, 1H) ppm. | MW: 533.67 MS (ESI) [M + 1]⁺: 534 | INT124/ 1 |
| 16 | 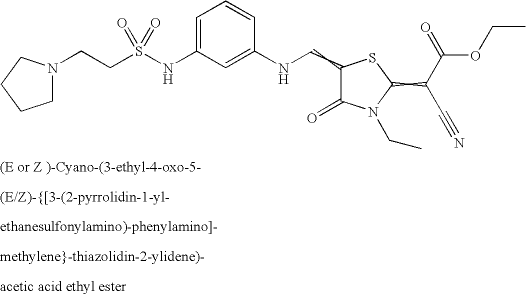 | 1.15-1.31(m,6H); 1.52-1.68 (m, 4H); 2.27-2.89 (m, 4H); 2.76 (t, 2H); 3.29 (t, 2H); 4.15-4.31 (m, 4H); 6.90 (d, 1H); 7.01 (d, 1H); 7.12 (s, 1H); 7.29 (t, 1H); 8.14 (s, 1H); 10.10-10.90 (b, 2H) ppm. | MW: 556.11 MS (ESI) [M + 1]⁺: 557 | INT124/ 1 |
| 17 | 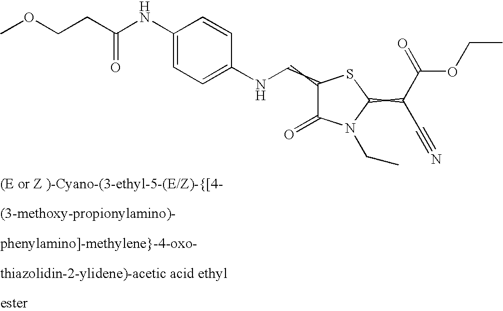 | 1.15-1.34 (m, 6H); 2.55 (t, 2H); 3.24 (s, 3H); 3.61 (t, 2H); 4.14-4.32 (m, 4H); 7.27 (d, 2H); 7.60 (d, 2H); 8.14 (s, 1H); 9.96 (s, 1H); 10.53 (s, 1H) ppm. | MW: 444.51 MS (ESI) [M + 1]⁺: 445 | INT124/ 1 |
| 18 | 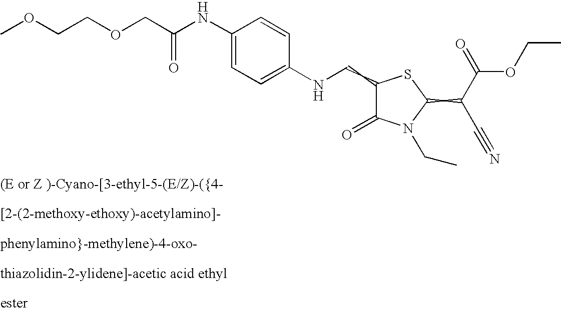 | 1.15-1.32 (m, 6H); 3.30 (s 3H); 3.52 (t, 2H); 3.67 (t, 2H); 4.08 (s, 2H); 4.17-4.32 (m, 4H); 7.29 (d, 2H); 7.63 (d, 2H); 8.15 (s, 1H); 9.67 (s, 1H); 10.53 (s, 1H) ppm. | MW: 474.54 MS (ESI) [M + 1]⁺: 475 | INT124/ 1 |
| 19 | 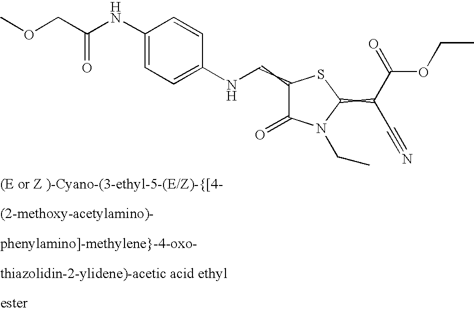 | 1.16-1.32 (m, 6H); 3.37 (s, 3H); 3.98 (s, 2H); 4.15-4.33 (m, 4H); 7.28 (d, 2H); 7.65 (d, 2H); 8.15 (s, 1H); 9.77 (s, 1H); 10.52 (s, 1H) ppm. | MW: 430.48 MS (ESI) [M + 1]⁺: 431 | INT124/ 1 |
| 20 | 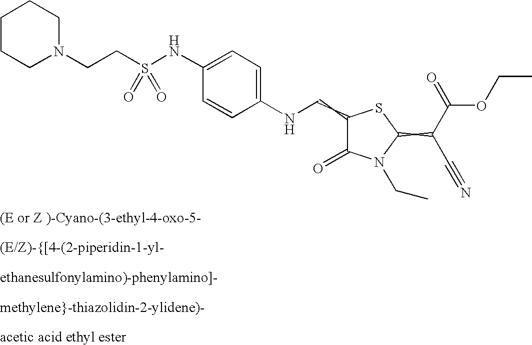 | 1.11-1.35(m,SH); 1.35-1.47(m,4H); 2.20-2.32 (m, 4H); 2.54 (t, 2H); 3.20 (t, 2H); 4.14-4.31 (m, 4H); 7.19 (d, 2H); 7.28 (d, 2H); 8.18 (s, 1H); 9.5-10.0 (b, 1H); 10.35-10.75 (b, 1H) ppm. | MW: 533.67 MS (ESI) [M + 1]⁺: 534 | INT124/ 1 |
| 21 | 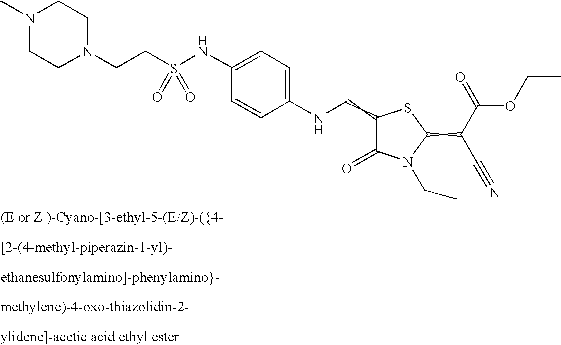 | 1.16-1.31 (m, 6H); 2.10 (5, 3H); 2.13-2.40 (m, 8H); 2.65 (t, 2H); 3.20 (t, 2H); 4.13-4.30 (m, 4H); 7.19 (d, 2H); 7.29 (d, 2H); 8.18 (s, 1H); 9.5-10.8 (b, 2H) ppm. | MW: 548.69 MS (ESI) [M + 1]⁺: 549 | INT124/ 1 |
| 22 | 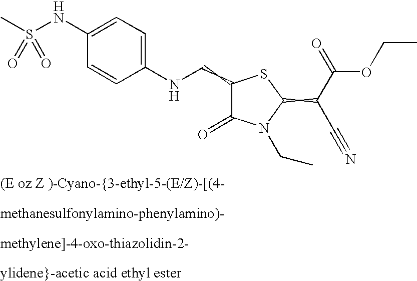 | 1.17-1.31 (m, 6H); 2.96 (s, 3H); 4.15-4.31 (m, 4H); 7.19 (d, 2H); 7.31 (d, 2H); 8.14 (s, 1H); 9.77 (s, 1H); 10.56 (s, 1H) ppm. | MW: 436.51 MS (ESI) [M + 1]⁺: 437 | INT124/ 1 |
| 23 | 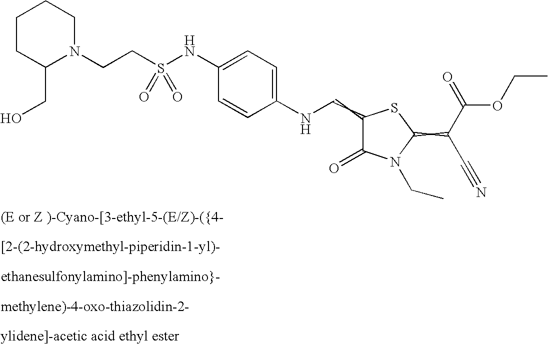 | 1.09-1.49 (m, 10H); 1.49-1.65 (m, 2H); 2.04-2.23 (m, 2H); 2.53-2.67 (m, 1H); 2.81-2.96 (m, 1H); 2.96-3.10 (m, 1H); 3.10-3.27 (m, 2H); 3.23-3.50 (m, 2H); 4.15-4.30 (m, 4H); 4.56 (s, 1H); 7.21 (d, 2H); 7.31 (d, 2H); 8.17 (s, 1H); 9.71 (s, 1H); 10.55 (s, 1H) ppm. | MW: 563.70 MS (ESI) [M + 1]⁺: 564 | INT124/ 1 |
| 24 | 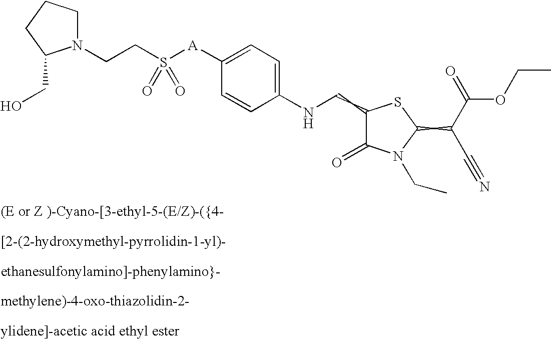 | 1.16-1.31 (m, 6H); 1.41-1.65 (m, 3H); 1.65-1.70 (m, 1H); 2.10-2.15 (m, 1H); 2.44 (m, 1H); 2.66 (m, 1H); 2.85 (m, 1H); 3.10-3.41 (m, 5H); 4.15-4.31 (m, 4H); 4.52 (s, 1H); 7.20 (d, 2H); 7.30 (d, 2H); 8.18 (s, 1H); 9.68 (s, 1H); 10.55 (s, 1H) ppm. | MW: 549.67 MS (ESI) [M + 1]⁺: 550 | INT124/ 1 |
| 25 | 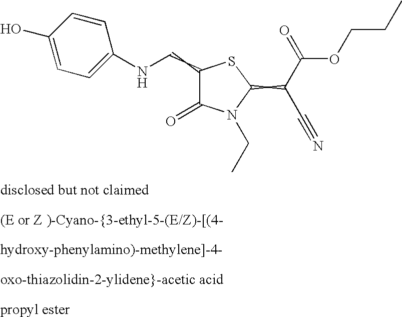 | 0.93 (t, 3H); 1.22 (t, 3H); 1.66 (sextet, 2H); 4.12 (t, 2H); 4.24 (q, 2H); 6.77 (d, 2H); 7.15 (d, 2H); 8.07 (s, 1H); 9.41 (s, 1H); 10.46(s, 1H) ppm. | MW: 373.43 MS (ESI) [M + 1]⁺: 374 | Educt as in the Case of INT124/ INT131 |
| 26 | 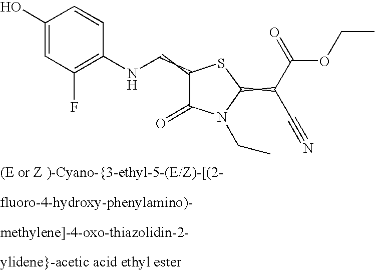 | 1.14-1.32(m, 6H); 4.10-4.34(m, 4H); 6.59-6.72 (m, 2H); 7.21 (t, 1H); 7.91 (s, 1H); 9.98 (s, 1H); 10.25 (s, b, 1H) ppm. | MW: 377.39 MS (ESI) [M + 1]⁺: 378 | INT124/ INT131 |
| 27 | 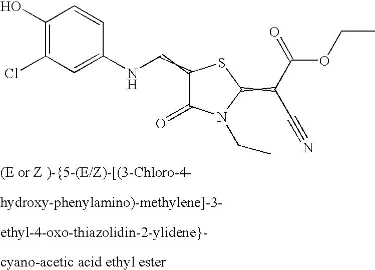 | 1.12-1.35 (m, 6H); 4.14-4.33 (m, 4H); 6.94 (d, 1H); 7.13 (d, 1H); 7.34 (s, 1H); 8.10 (s, 1H); 10.10 (s, 1H); 10.40 (s, 1H) ppm. | MW: 393.85 MS (ESI) [M + 1]⁺: 394 | INT124/ INT131 |
| 28 | 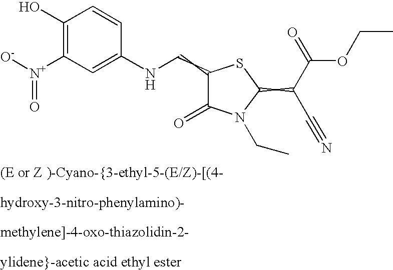 | 1.16-1.32 (m, 6H); 4.15-4.32 (m, 4H); 7.10 (d, 1H); 7.56 (dd, 1H); 7.84 (d, 1H); 8.18 (s, 1H); 10.10-10.70 (b, 2H) ppm. | MW: 404.40 MS (ESI) [M + 1]⁺: 405 | INT124/ INT131 |
| 29 | 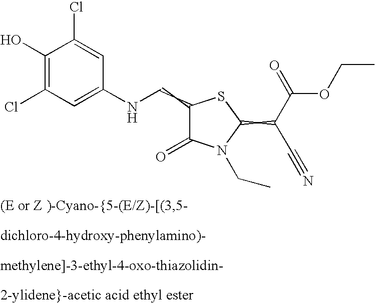 | 1.15-1.31 (m, 6H); 4.12-4.31 (m, 4H); 7.31 (m, 2H); 8.15 (s, 1H); 10.10-10.60 (b, 2H) ppm. | MW: 428.29 MS (ESI) [M + 1]⁺: 429 | INT124/ INT131 |
| 30 | 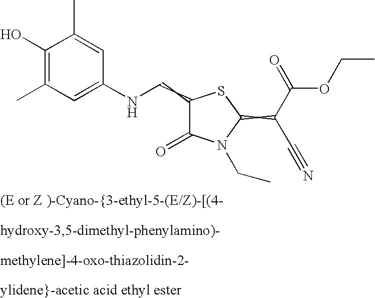 | 1.17-1.31 (m, 6H); 2.17 (s, 6H); 4.12-4.31 (m, 4H); 6.90 (s, 2H); 8.08 (s, 1H); 8.20 (s, 1H); 10.38 (s, 1H) ppm. | MW: 387.46 MS (ESI) [M + 1]⁺: 388 | INT124/ INT131 |
| 31 | 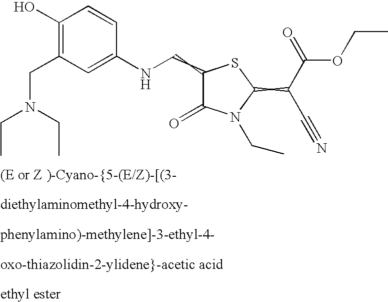 | 1.01 (t, 6H); 1.15-1.34 (m, 6H); 2.55 (q, 4H); 3.70 (s, 2H); 4.13-4.31 (m, 4H); 6.68 (d, 1H); 7.02 (d, 1H); 7.09 (s, 1H); 8.08 (s, 1H); 10.45 (s, 1H) ppm. | MW: 444.55 MS (ESI) [M + 1]⁺: 445 | INT124/ INT132 |
| 32 | 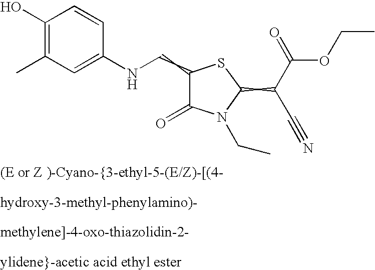 | 1.18-1.31 (m, 6H); 2.12 (s, 3H); 4.15-4.30 (m, 4H); 6.75 (d, 1H); 6.95 (d, 1H); 7.07 (s, 1H); 8.06 (d, 1H); 9.30 (s, 1H); 10.40 (d, 1H) ppm. | MW: 373.43 MS (ESI) [M + 1]⁺: 374 | INT124/ INT131 |
| 33 | 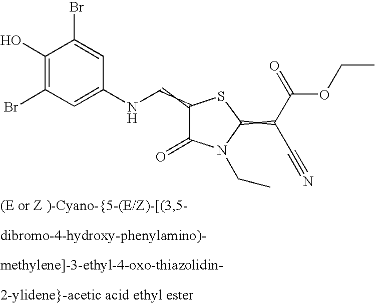 | 1.18-1.32 (m, 6H); 4.14-4.30 (m, 4H); 7.46 (m, 3H); 8.12 (s, 1H); 10.50 (s, b, 1H) ppm. | MW: 517.20 MS (ESI) [M + 1]⁺: 518 | INT124/ INT131 |
| 34 | 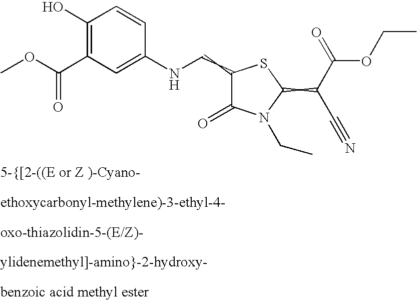 | 1.18-1.30 (m, 6H); 3.90 (s, 3H); 4.15-4.30 (m, 4H); 7.00 (d, 1H); 7.51 (d, 1H); 7.64 (s, 1H); 8.12 (s, 1H); 10.28 (s, 1H); 10.52 (s, 1H) ppm. | MW: 417.44 MS (ESI) [M + 1]⁺: 418 | INT124/ INT131 |
| 35 | 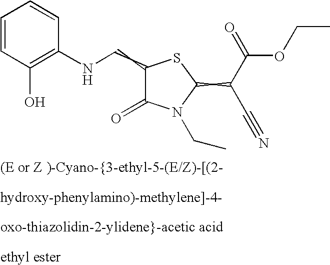 | 1.17-1.31 (m, 6H); 4.13-4.35 (m, 4H); 6.78-7.02 (m, 3H); 7.40 (d, 1H); 8.60 (s, 1H); 10.20 (b, 2H) ppm. | MW: 359.40 MS (ESI) [M + 1]⁺: 360 | INT124/ INT131 |
| 36 | 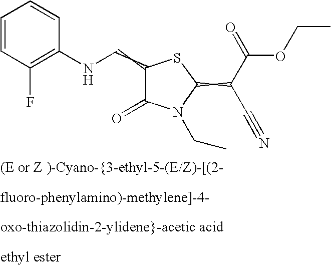 | Main Isomer: 1.16-1.32 (m, 6H); 4.15-4.32 (m, 4H); 7.10-7.60 (m, 4H); 8.06 (d, 1H); 10.49 (d, 1H) ppm. | MW: 361.40 MS (ESI) [M + 1]⁺: 362 | INT124/ INT131 |
| 37 | 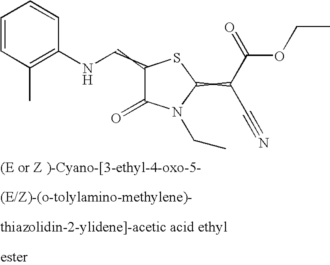 | Main Isomer: 1.17-1.33 (m, 6H); 2.30 (s, 3H); 4.13-4.33 (m, 4H); 7.01-7.47 (m, 4H); 7.92 (s, 1H); 10.00 (s, 1H) ppm. | MW: 357.43 MS (ESI) [M + 1]⁺: 358 | INT124/ INT131 |
| 38 | 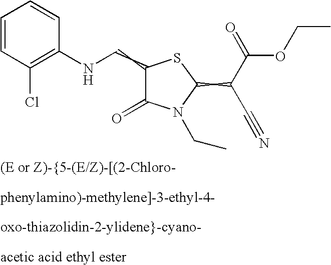 | MW: 377.85 MS (ESI) [M + 1]⁺: 378 | INT124/ INT131 | |
| 39 | 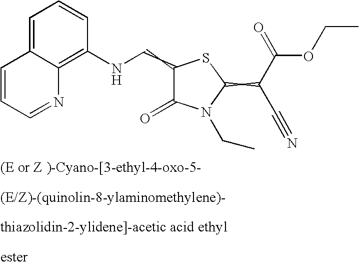 | CDCl₃: 1.38 (t, 3H); 1.46 (t, 3H); 4.33 (q, 2H); 4.51 (q, 2H); 7.40-7.59 (m, 4H); 7.87 (d, 1H); 8.18 (d, 1H); 9.00 (m, 1H); 12.26 (d, 1H) ppm. | MW: 394.45 MS (ESI) [M + 1]⁺: 395 | INT124/ INT131 |
| 40 | 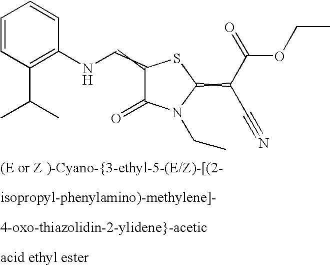 | Main Isomer: 1.10-1.36(m, 12H); 3.03-3.18 (m, 1H); 4.11-4.33 (m, 4H) 7.10-7.47 (m, 4H); 7.89 (s, 1H); 10.12 (s, 1H) ppm. | MW: 385.49 MS (ESI) [M + 1]⁺: 386 | INT124/ INT131 |
| 41 | 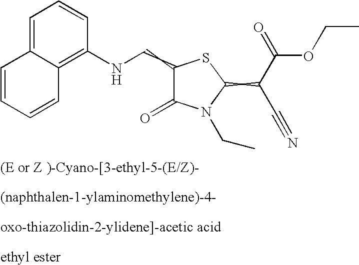 | Main Isomer: 1.16-1.35 (m, 6H); 4.12-4.35 (m, 4H); 7.44 (d, 1H); 7.50-7.68 (m, 3H); 7.85 (d, 1H); 7.94-8.05 (m, 1H); 8.05-8.20 (m, 2H); 10.73 (s, 1H) ppm. | MW: 393.47 MS (ESI) [M + 1]⁺: 394 | INT124/ INT131 |
| 42 | 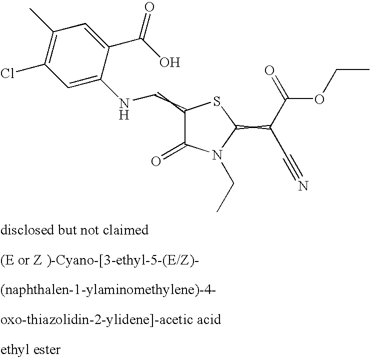 | Main Isomer: 1.16-1.45 (m,6H); 4.13-4.32 (m, 4H); 7.12-7.23 (m, 1H); 7.80 (s, 1H); 7.92-8.01 (m, 1H); 8.59 (d, 1H); 12.60 (d, 1H); 13.5-14.0 (b, 1H) ppm. | MW: 421.86 MS (ESI) [M + 1]^{+: 422} | INT124/ INT131 |
| 43 | 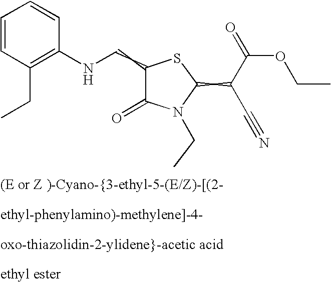 | Main Isomer: 1.10-1.32 (m, 9H); 2.70 (q, 2H); 4.12-4.33 (m, 4H); 7.17-7.47 (m, 4H); 7.90 (s, 1H); 10.03 (s, b, 1H) ppm. | MW: 371.46 MS (ESI) [M + 1]⁺: 372 | INT124/ INT131 |
| 44 | 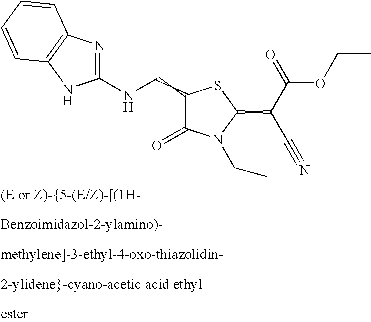 | 1.17-1.31 (m, 6H); 4.13-4.32 (m, 4H); 7.19 (m, 2H); 7.30 (m, 2H); 8.63 (s, 1H); 12.74 (s, 2H) ppm. | MW: 383.43 MS (ESI) [M + 1]⁺: 384 | INT124/ INT131 |
| 45 | 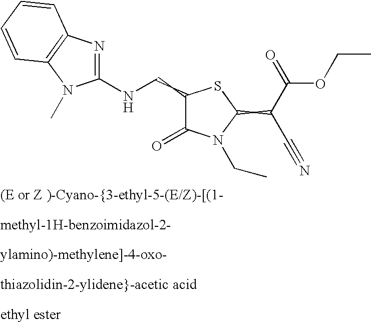 | 1.28-1.31 (m, 6H); 3.63 (s, 3H); 4.12-4.30 (m, 4H); 7.18 (m, 2H); 7.31 (m, 1H); 7.46 (m, 1H); 8.60 (s, 1H); 12.91 (s, 1H) ppm. | MW: 397.46 MS (ESI) [M + 1]⁺: 398 | INT124/ INT131 |
| 46 | 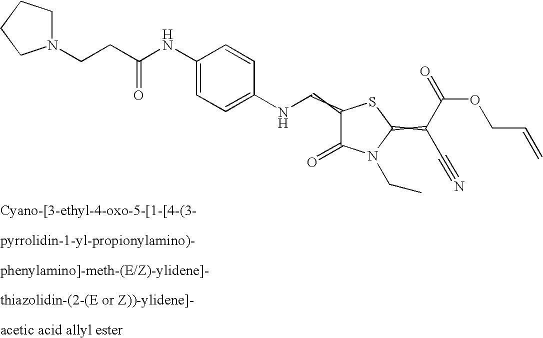 | | MW: 495.60 MS (ESI) [M + 1]⁺: 496 | INT126/ 3 |
| 47 | 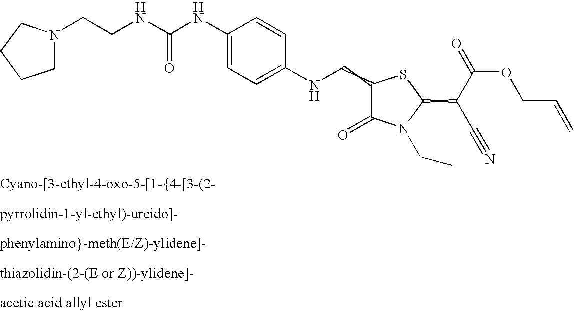 | | MW: 510.62 MS (ESI) [M + 1]⁺: 511 | INT126/ 3 |
| 48 | 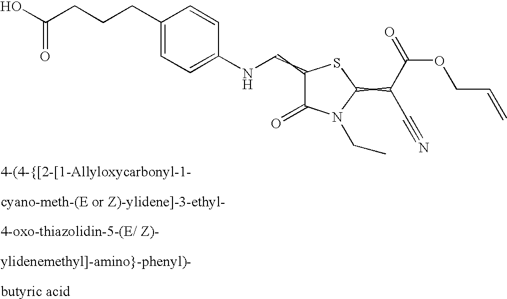 | | MW: 441.51 MS (ESI) [M + 1]⁺: 442 | INT126/ 3 |
| 49 | 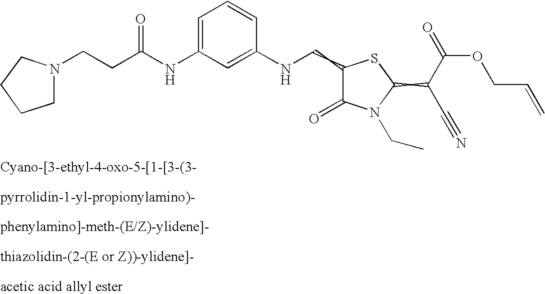 | | MW: 495.60 MS (ESI) [M + 1]⁺: 496 | INT126/ 3 |
| 50 | 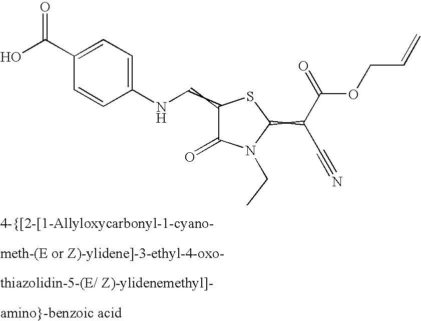 | | MW: 399.43 MS (ESI) [M + 1]⁺: 400 | INT126/ 3 |
| 51 | 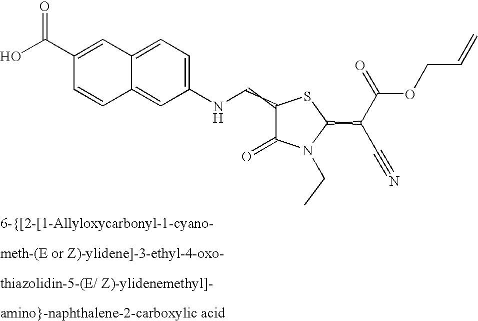 | | MW: 449.49 MS (ESI) [M + 1]⁺: 450 | INT126/ 3 |
| 52 | 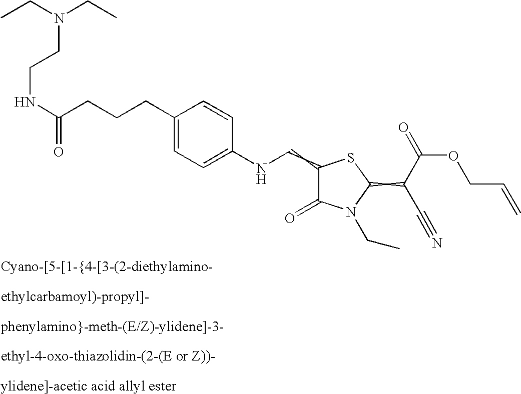 | | MW: 539.70 MS (ESI) [M + 1]⁺: 540 | 48/5 |
| 53 | 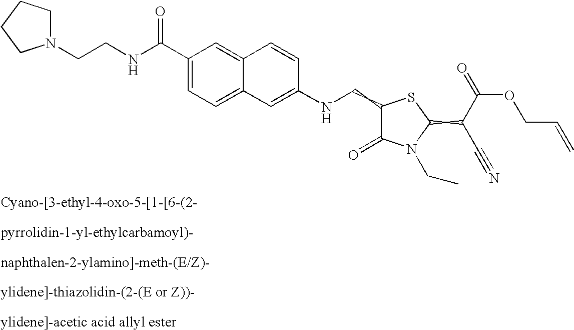 | | MW: 545.67 MS (ESI) [M + 1]⁺: 546 | 51/5 |
| 54 | 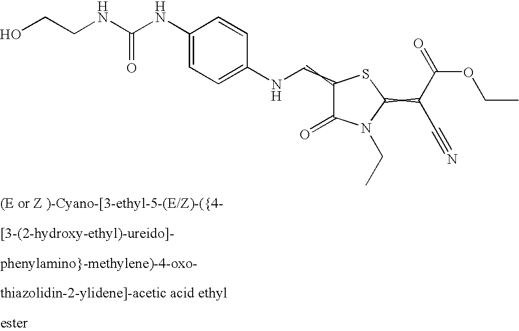 | 1.24 (m, 6H), 3.12 (m, 2H), 3.42 (m, 2H), 4.20 (m, 4H), 4.72 (m, 1H), 6.13 (m, 1H), 7.21 (d, 2H), 7.38 (d, 2H), 8.12 (m, 1H), 8.59 (s, 1H), 10.50 (s, 1H). | MW: 445.50 MS (ESI) [M + 1]⁺: 446 | INT124/ 2 |
| 55 | 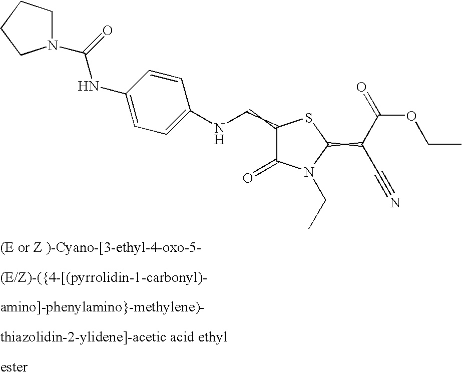 | 1.21 (m, 6H), 1.81 (m, 4H), 3.32 (m, 4H), 4.20 (m, 2H), 7.18 (d, 2H), 7.50 (d, 2H), 8.12 (s, 1H) | MW: 455.54 MS (ESI) [M + 1]⁺: 456 | INT124/ 2 |
| 56 | 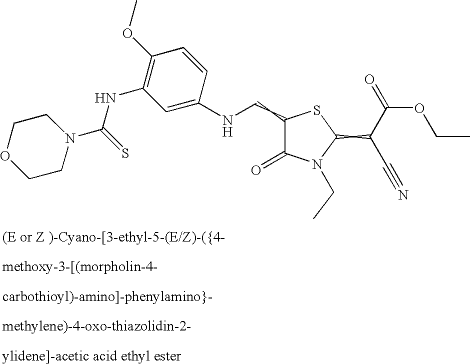 | 1.28 (m, 6H), 3.63 (m, 4H), 3.38 (s, 3H), 3.90 (m, 4H), 4.21 (m, 4H), 7.0 (d, 1H), 7.16 (dd, 1H), 7.30 (d, 1H), 8.08 (m, 1H), 8.89 (d, 1H), 10.50 (d, 1H). | MW: 517.63 MS (ESI) [M + 1]⁺: 518 | INT124/ 2 |
| 57 | 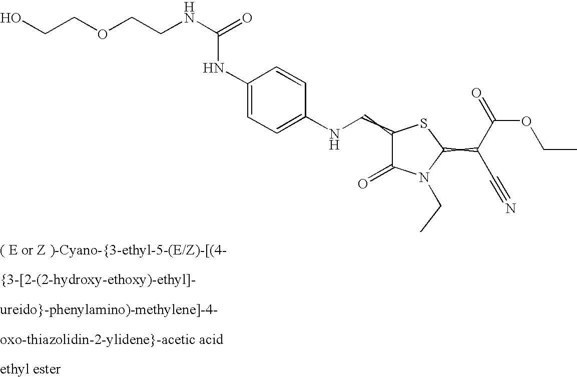 | 1.22 (m, 6H), 3.22 (m, 2H), 3.41 (m, 4H), 3.53 (m, 2H), 4.21 (m, 4H), 4.60 (m, 1H), 6.16 (m, 1H), 7.20 (d, 2H), 7.38 (d, 2H), 8.10 (s, 1H), 8.58 (s, 1H), 10.50 (s, 1H). | MW: 489.55 MS (ESI) [M + 1]⁺: 490 | INT124/ 2 |
| 58 | 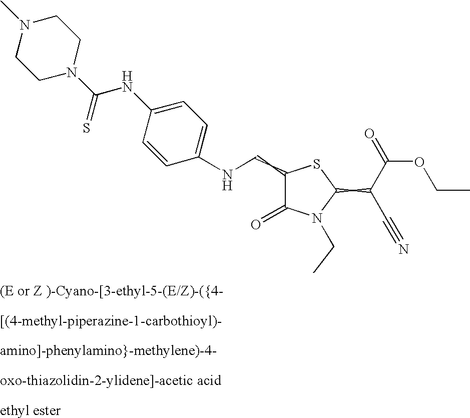 | 1.22 (m, 6H), 2.20 (s, 3H), 2.35 (m, 4H), 3.82 (m, 4H), 4.21 (m, 4H), 7.22 (m, 4H), 8.14 (s, 1H), 9.28 (s, 1H), 10.55 (s, 1H). | MW: 500.65 MS (ESI) [M + 1]⁺: 501 | INT124/ 2 |
| 59 | 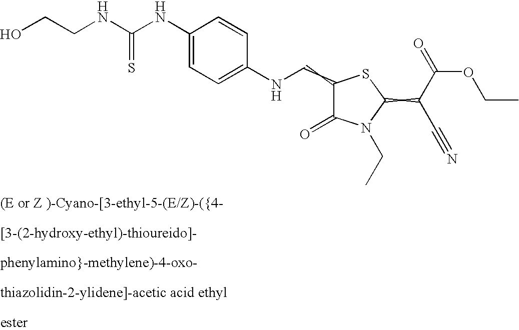 | 1.27 (m, 6H), 3.51 (m, 4H), 4.22 (m, 4H), 4.81 (s, 1H), 7.27 (d, 2H), 7.40 (d, 2H), 7.68 (s, 1H), 8.13 (d, 1H), 9.59 (s, 1H), 10.55 (d, 1H) | MW: 461.57 MS (ESI) [M + 1]⁺: 462 | INT124/ 2 |
| 60 | 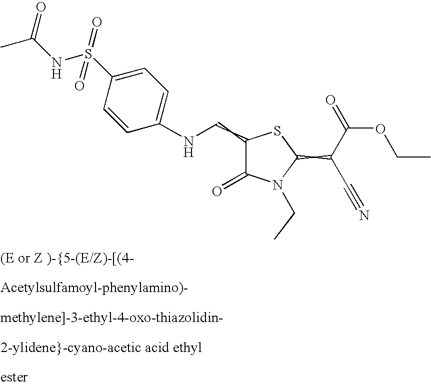 | 1.25 (m, 6H), 1.88 (m, 3H), 4.24 (m, 4H), 7.52 (d, 2H), 7.87 (d, 2H), 8.26 (d, 1H), 10.78 (d, 1H), 12.00 (s, 1H). | MW: 464.52 MS (ESI) [M + 1]⁺: 465 | INT124/ 2 |
| 61 | 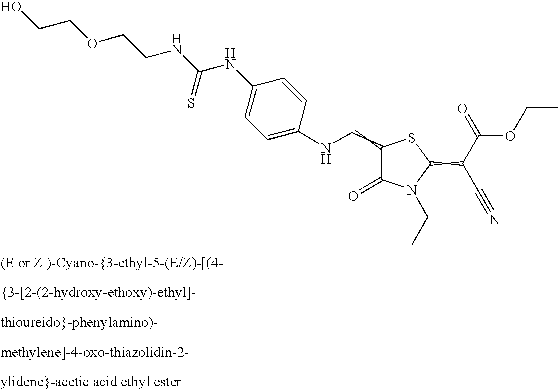 | 1.24 (m, 6H), 3.50 (m, 8H), 4.21 (m, 4H), 4.60 (m, 1H), 7.27 (d, 2H), 7.40 (d, 2H), 7.70 (s, 1H), 8.17 (s, 1H), 9.58 (s, 1H), 10.52 (s, 1H). | MW: 505.62 MS (ESI) [M + 1]^{+: 506} | INT124/ 2 |
| 62 | 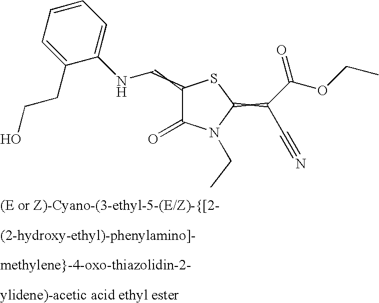 | 1.22 (m, 6H), 2.81 (m, 2H), 3.69 (m, 2H), 4.21 (m, 4H), 7.29 (m, 4H), 8.00 (s, 1H). | MW: 387.46 MS (ESI) [M + 1]⁺: 388 | INT124/ 2 |
| 63 | 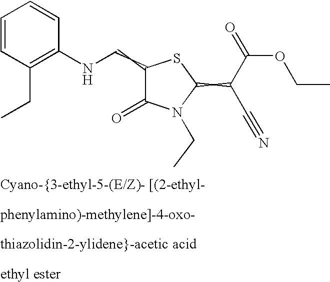 | 1.27 (m, 9H), 2.68 (m, 2H), 4.22 (m, 4H), 7.27 (m, 4H), 7.88 (s, 1H). | MW: 371.46 MS (ESI) [M + 1]⁺: 372 | INT124/ 2 |
| 64 | 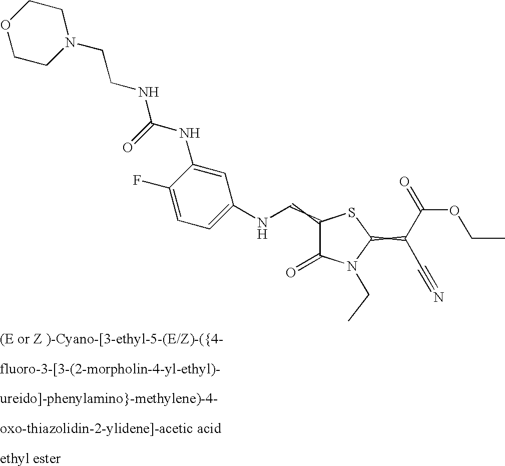 | 1.25 (m, 6H), 2.40 (m, 6H), 3.26 (m, 2H), 3.58 (m, 4H), 4.22 (m, 4H), 6.70 (m, 1H), 6.84 (m, 1H), 7.18 (m, 1H), 8.02 (s, 1H), 8.19 (dd, 1H), 8.57 (d, 1H), 10.62 (s, 1H). | MW: 532.59 MS (ESI) [M + 1]⁺: 533 | INT124/ 2 |
| 65 | 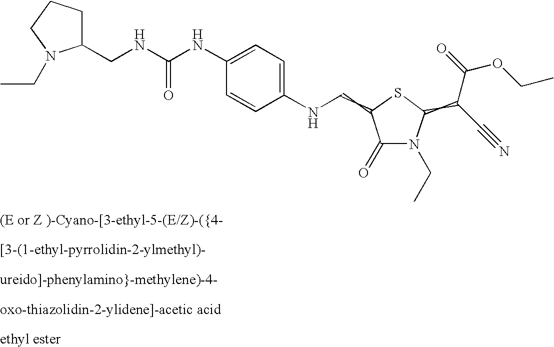 | 1.05 (m, 3H), 1.22 (m, 6H), 1.52 (m, 1H), 1.66 (m, 2H), 1.80 (m, 1H), 2.16 (m, 2H), 2.49 (m, 1H), 2.80 (m, 1H), 2.97 (m, 1H), 3.08 (m, 1H), 3.38 (m, 1H), 4.20 (m, 4H), 6.00 (m, 1H), 7.20 (d, 2H), 7.48 (d, 2H), 8.09 (s, 1H), 8.22 (s, 1H), 10.50 (s, 1H). | MW: 512.63 MS (ESI) [M + 1]⁺: 513 | INT124/ 2 |
| 66 | 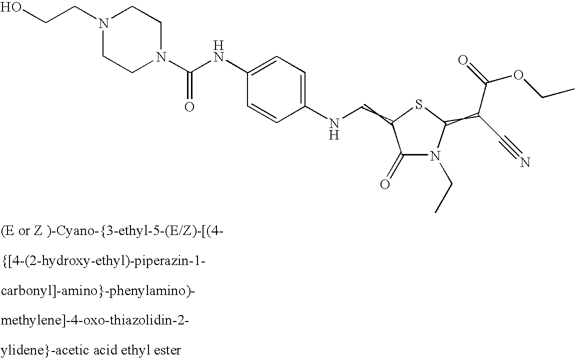 | 1.21 (m, 6H), 2.40 (m, 4H), 3.50 (m, 2H), 4.21 (m, 4H), 4.42 (s, 1H), 7.20 (d, 2H), 7.45 (d, 2H), 8.12 (s, 1H), 8.50 (s, 1H). | MW: 514.60 MS (ESI) [M + 1]⁺: 515 | INT124/ 2 |
| 67 | 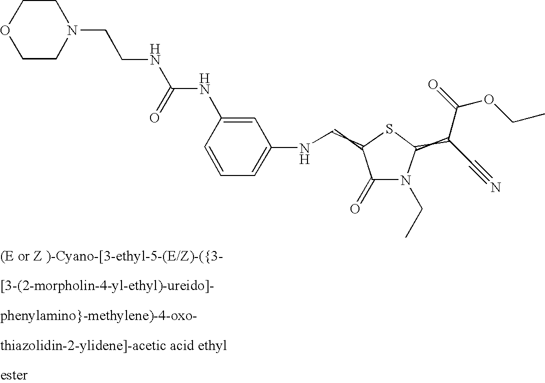 | 1.22 (m, 6H), 2.39 (m, 6H), 3.21 (m, 2H), 3.58 (m, 4H), 4.21 (m, 4H), 6.11 (m, 1H), 6.81 (dd, 1H), 6.93 (dd, 1H), 7.19 (m, 1H), 7.58 (s, 1H), 8.08 (m, 1H), 8.72 (d, 1H), 10.59 (d, 1H). | MW: 514.60 MS (ESI) [M + 1]⁺: 515 | INT124/ 2 |
| 68 | 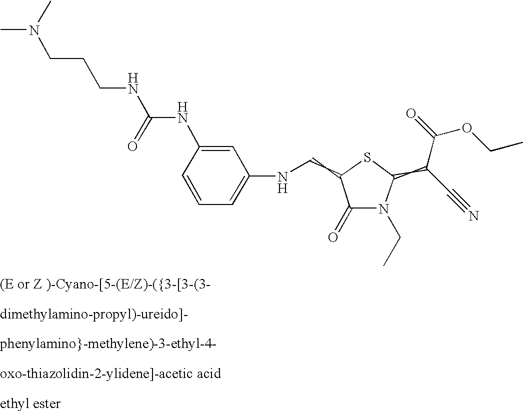 | 1.24 (m, 6H), 1.57 (m, 2H), 2.12 (s, 6H), 2.25 (m, 2H), 3.11 (m, 2H), 4.21 (m, 4H), 6.20 (m, 1H), 6.80 (d, 1H), 6.92 (d, 1H), 7.18 (m, 1H), 7.57 (s, 1H), 8.09 (s, 1H), 8.57 (s, 1H). | MW: 486.59 MS (ESI) [M + 1]⁺: 487 | INT124/ 2 |
| 69 | 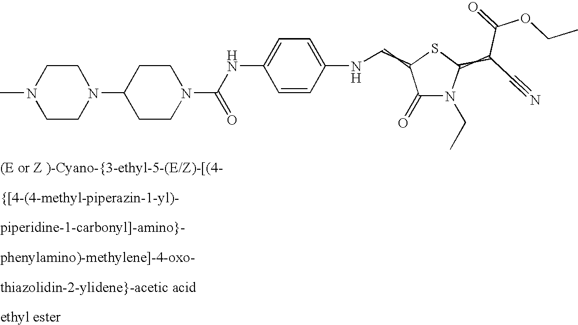 | 1.22 (m, 6H), 1.41 N (m, 2H), 1.70 (m, 2H), 1.83 (m, 2H), 2.15 (s, 3H), 2.48 (m, 3H), 2.79 (m, 2H), 3.37 (m, 6H), 4.21 (m, 4H), 7.20 (d, 2H), 7.42 (d, 2H), 8.12 (s, 1H), 8.50 (s, 1H). | MW: 567.71 MS (ESI) [M + 1]^{+: 568} | INT124/ 2 |
| 70 | 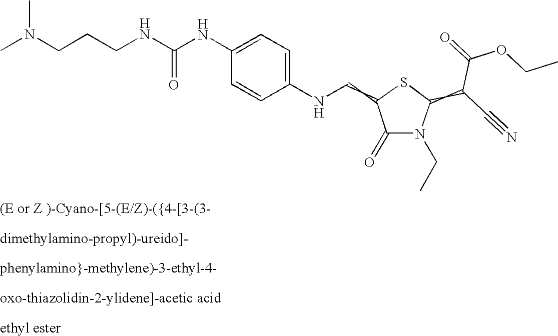 | 1.22 (m, 6H), 1.53 (m, 2H), 2.12 (s, 6H), 2.25 (m, 2H), 3.09 (m, 2H), 4.22 (m, 4H), 6.12 (m, 1H), 8.10 (s, 1H), 8.48 (s, 1H). | MW: 486.59 MS (ESI) [M + 1]^{+: 487} | INT124/ 2 |
| 71 | 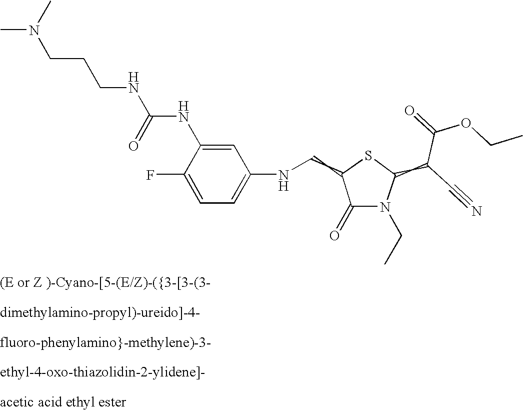 | 1.22 (m, 6H), 1.58 (m, 2H), 2.12 (s, 6H), 2.25 (m, 2H), 3.12 (m, 2H), 4.21 (m, 4H), 6.70 (m, 1H), 6.83 (m, 1H), 7.16 (m, 1H), 8.06 (s, 1H), 8.19 (m, 1H), 8.39 (s, 1H). | MW: 504.58 MS (ESI) [M + 1]⁺: 505 | INT124/ 2 |
| 72 | 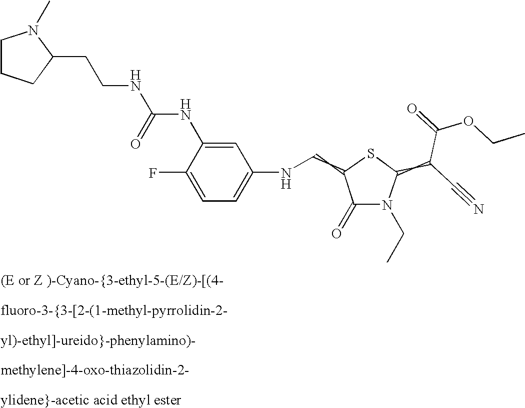 | 1.28 (m, 6H), 1.41 (m, 2H), 1.62 (m, 2H), 1.76 (m, 1H), 1.91 (m, 1H), 2.08 (m, 2H), 2.22 (s, 3H), 2.93 (m, 1H), 3.12 (m, 2H), 4.21 (m, 4H), 6.68 (m, 1H), 6.82 (m, 1H), 7.17 (m, 1H), 10.59 (s, 1H). | MW: 530.62 MS (ESI) [M + 1]⁺: 531 | INT124/ 2 |
| 73 | 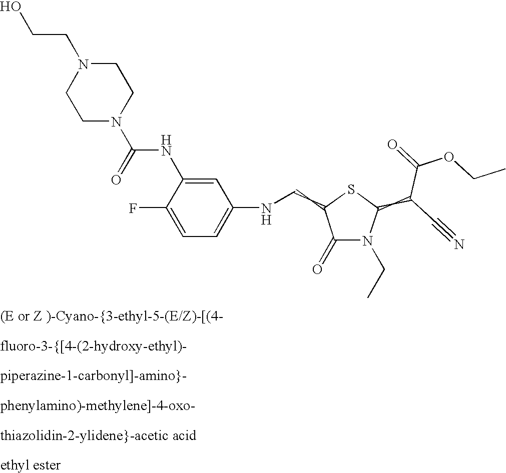 | In MeOH: 1.32 (m, 6H), 2.60 (m, 6H), 3.59 (m, 4H), 3.70 (m, 2H), 4.30 (m, 4H), 6.89 (m, 1H), 7.08 (m, 1H), 7.38 (m, 1H), 8.05 (s, 1H). | MW: 532.59 MS (ESI) [M + 1]⁺: 533 | INT124/ 2 |
| 74 | 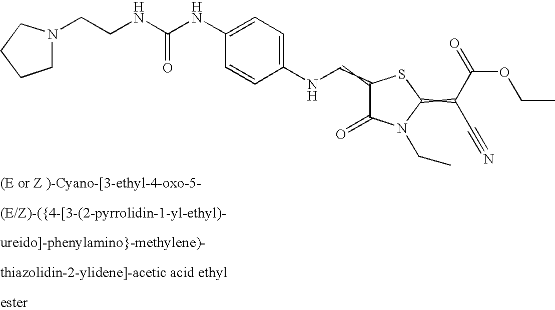 | 1.21 (m, 6H), 1.70 (m, 4H), 3.18 (m, 4H), 4.21 (m, 4H), 6.08 (m, 1H), 7.19 (d, 2H), 7.38 (d, 2H), 8.10 (s, 1H), 8.65 (s, 1H), 10.50 (s, 1H). | MW: 498.61 MS (ESI) [M + 1]^{+: 499} | INT124/ 2 |
| 75 | 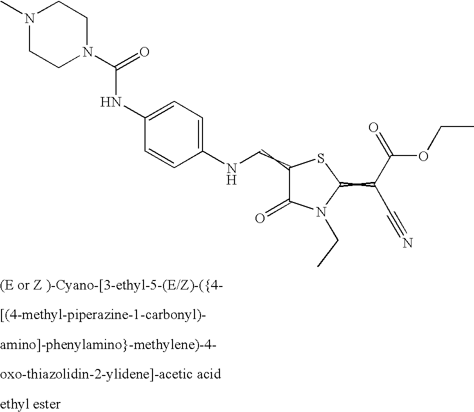 | 1.22 (m, 6H), 2.17 (s, 3H), 2.30 (m, 4H), 3.40 (m, 4H), 4.22 (m, 4H), 7.20 (d, 2H), 7.42 (d, 2H), 8.11 (s, 1H), 8.51 (s, 1H), 10.40 (s, 1H). | MW: 484.58 MS (ESI) [M + 1]⁺: 485 | INT124/ 2 |
| 76 | 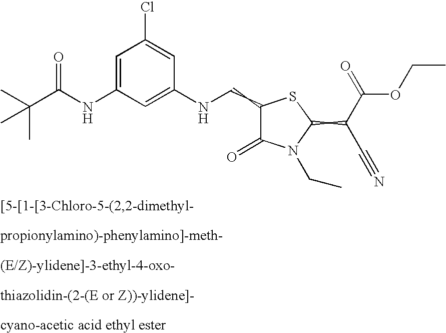 | (DMSO-d6, Stored with K₂CO₃, Main Isomer): δ =1.17-1.30 (m, 15H); 4.16-4.30 (m, 4H); 7.01 (s, 1H); 7.51 (s, 1H); 7.63 (s, 1H); 8.15 (s, 1H); 9.33 (s, 1H); 10.60 (s, 1H) ppm. | MW: 476.98 MS (ESI) [M + 1]⁺: 477 | INT124/ INT131 |
| 77 | 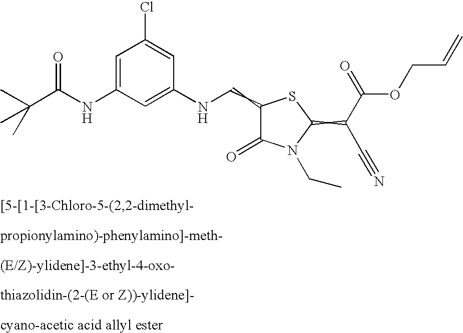 | (DMSO-d6, Stored with K₂CO₃, Main Isomer): δ =1.17-1.3 1 (m, 12H); 4.26 (q, 2H); 4.72 (d, 1H); 5.26 (d, 1H); 5.38 (d, 1H); 5.91-6.08 (m, 1H); 7.06 (s, 1H); 7.52 (s, 1H); 7.70 (s, 1H); 8.13 (s, 1H); 9.38 (s, 1H); 10.61 (s, 1H) ppm. | MW: 488.99 MS (ESI) [M + 1]⁺: 489 | INT126/ INT131 |
| 78 | 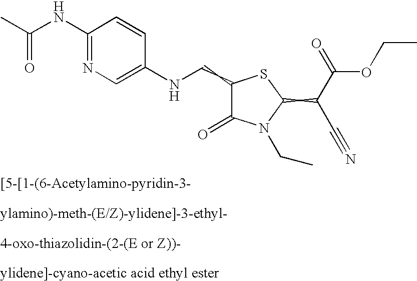 | (DMSO-d6, Stored with K₂CO₃, Main Isomer): δ =1.18-1.33 (m, 6H); 2.08 (s, 3H); 4.15-4.33 (m, 4H); 7.78 (dd, 1H); 8.08 (d, 1H); 8.20 (s, 1H); 8.31 (d, 1H); 10.49 (s, 1H); 10.55 (s, 1H) ppm. | MW: 401.45 MS (ESI) [M + 1]⁺: 402 | INT124/ INT131 |
| 79 | 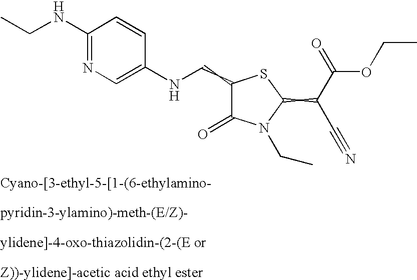 | (DMSO-d6, Stored with K₂CO3_{, Main Isomer): δ =1.12 (t, 3H); 1.18-1.32 (m, 6H); 3.23 (m, 2H); 4.13-4.32 (m, 4H); 6.42-6.59 (m, 2H); 7.45 (m, 1H); 7.94-8.06 (m, 2H); 10.40 (s, 1H) ppm.} | MW: 387.46 MS (ESI) [M + 1]⁺: 388 | INT124/ INT131 |
| 80 | 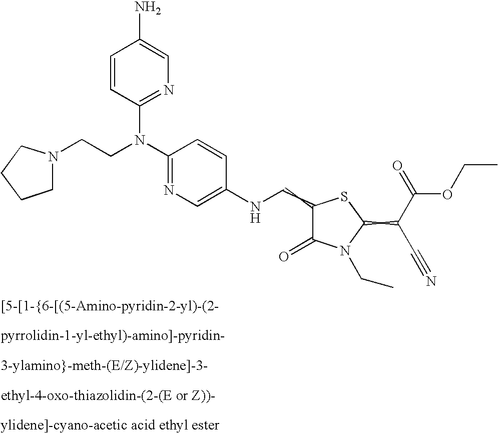 | (DMSO-d6, Stored with K₂CO₃, Main Isomer): δ =1.18-1.31 (m, 6H); 1.83 (m, 4H); 2.80-3.21 (m, 6H); 4.08-4.32 (m, 6H); 5.37 (s, 2H); 6.58 (d, 1H); 7.04 (m, 2H); 7.55 (m, 1H); 7.83 (s, 1H); 8.10 (s, 1H); 8.22 (s, 1H); 10.46 (s, 1H) ppm. | MW: 548.67 MS (ESI) [M + 1]⁺: 549 INT124/ INT132 | |
| 81 | 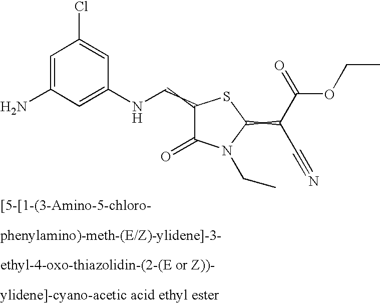 | (DMSO-d6, Stored with K₂CO₃, Main Isomer): 1.02-1.30 (m, 6H); 4.14-4.30 (m, 4H); 5.50 (s, b, 2H); 6.29 (s, 1H); 6.37 (s, b, 2H); 8.09 (s, 1H); 10.40 (s, 1H) ppm. | MW: 392.87 MS (ESI) [M + 1]⁺: 393 | INT124/ INT131 |
| 82 | 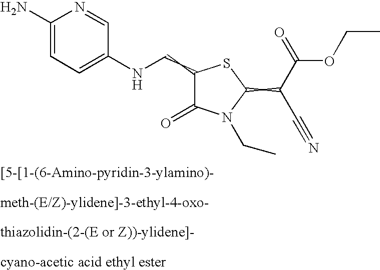 | (DMSO-d6, Stored with K₂CO₃, Main Isomer): δ =1.17-1.32 (m, 6H); 4.13-4.32 (m, 4H); 5.44 (s, 2H); 6.47 (d, 1H); 7.44 (d, 1H); 7.92 (s, 1H); 8.03 (s, 1H); 10.38 (s, 1H) ppm. | MW: 359.41 MS (ESI) [M + 1]⁺: 360 | INT124/ INT132 |
| 83 | 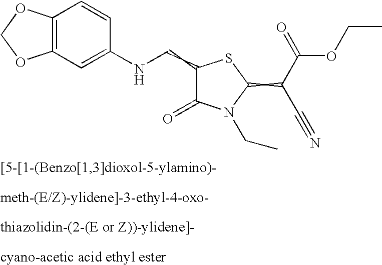 | (DMSO-d6, Stored with K₂CO₃, Main Isomer): δ =1.07-1.33 (m, 6H); 4.17-4.31 (m, 4H); 6.02 (s, 2H); 6.77 (dd, 1H); 6.90 (d, 1H); 7.03 (d, 1H); 8.10 (s, 1H); 10.42 (s, 1H) ppm. | MW: 387.41 MS (ESI) ]M + 1]⁺: 388 | INT124/ INT131 |
| 84 | 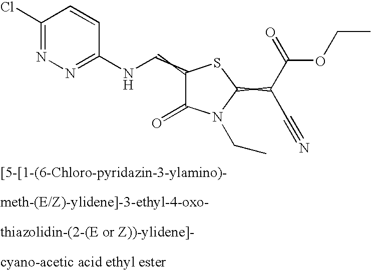 | (DMSO-d6, Stored with K₂CO₃, Main Isomer): δ =1.20-1.32 (m, 6H); 4.19-4.32 (m, 4H); 743 (d, 1H); 7.80 (d, 1H); 8.72 (s, 1H); 11.17 (s, 1H) ppm. | MW: 379.83 MS (ESI) [M + 1]⁺: 380 | INT124/ INT131 |
| 85 | 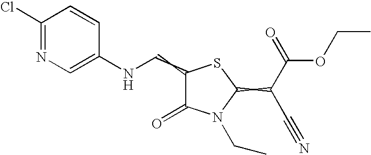 | (DMSO-d6, Stored with K₂CO₃, Main Isomer): δ =1.19-1.32 (m, 6H); 4.18-4.31 (m,4H); 7.47 (d, 1H); 7.87 (dd, 1H); 8.24 (s, 1H); 8.41 (d, 1H); 10.58 (s, 1H) ppm. | MW: 378.84 MS (ESI) [M + 1]^{+: 379} | INT124/ INT131 |
| 86 | 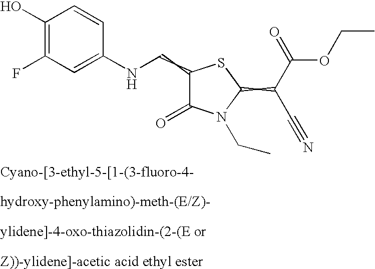 | (DMSO-d6, Stored with K₂CO₃, Main Isomer): δ =1.22 (b, 6H); 4.24 (b, 4H); 6.70-7.50 (m, 3H); 8.10 (s, b, 1H); 9.79 (s, b, 1H); 10.43 (s, b, 1H) ppm. | MW: 377.39 MS (ESI) [M + 1]⁺: 378 | INT124/ INT131 |
| 87 | 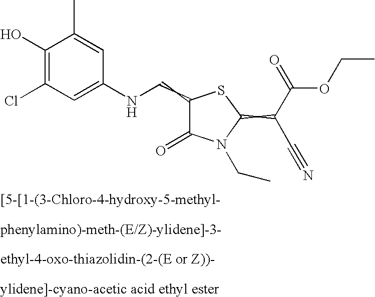 | (DMSO-d6, Stored with K₂CO₃, Main Isomer): δ =1.17-1.31 (m, 6H); 2.30 (s, 3H); 4.14-4.30 (m, 4H); 7.11 (d, 1H); 7.19 (d, 1H); 8.12 (s, 1H); 9.07 (s, 1H); 10.46 (s, 1H) ppm. | MW: 407.88 MS (ESI) [M + 1]⁺: 408 | INT124/ INT131 |
| 88 | 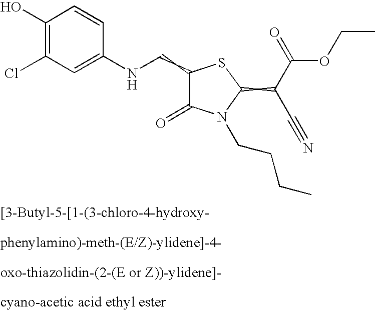 | (DMSO-d6, Stored with K₂CO₃, Main Isomer): δ =0.92 (t, 3H); 1.27 (t, 3H); 1.33 (m, 2H); 1.62 (m, 2H); 4.12‥4.30 (m, 4H); 6.95 (d, 1H); 7.13 (dd, 1H); 7.33 (d, 1H); 8.10 (s, 1H); 10.09 (s, 1H); 10.40(s, 1H) ppm. | MW: 421.90 MS (ESI) [M + 1]⁺: 422 | INT130/ INT132 |
| 89 | 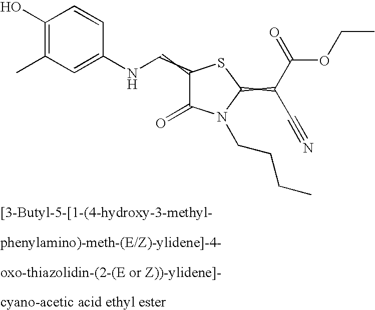 | (DMSO-d6, Stored with K₂CO₃, Main Isomer): δ =0.91 (t, 3H); 1.26 (t, 3H); 1.32 (m, 2H); 1.61 (m, 2H); 2.11 (s,3H); 4.12-4.28 (m, 4H); 6.64 (d, 1H); 6.92 (d, 1H); 7.23 (s, 1H); 8.09 (s, 1H); 9.23 (s, 1H); 10.42 (s, 1H) ppm. | MW: 401.49 MS (ESI) [M + 1]⁺: 402 | INT130/ INT132 |
| 90 | 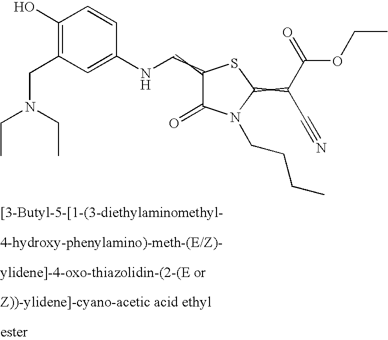 | (CDCl₃, Stored with K₂CO₃, Main Isomer): δ =0.99 (t, 3H); 1.11 (t, 6H); 1.36 (t, 3H); 1.45 (m, 2H); 1.76 (m, 2H); 2.63 (q, 4H); 3.77 (s, 2H); 4.25-4.46 (m, 4H); 6.72 (m, 1H); 6.76-6.97 (m, 2H); 7.50 (d, 1H); 10.54 (d, 1H) ppm. | MW: 472.61 MS (ESI) [M + 1]⁺: 473 | INT130/ INT132 |
| 91 | 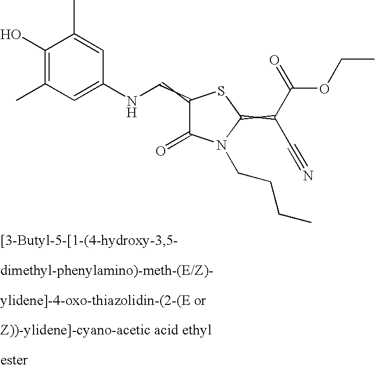 | (DMSO-d6, Stored with K₂CO₃, Main Isomer): δ =0.92 (t, 3H); 1.26 (t, 3H); 1.32 (m, 2H); 1.61 (m, 2H); 2.27 (s, 6H); 4.12-4.28 (m, 4H); 6.91 (s, 2H); 8.08 (s, 1H); 8.21 (s, 1H); 10.39 (s, 1H) ppm. | MW: 415.51 MS (ESI) [M + 1]⁺: 416 | INT130/ INT132 |
| 92 | 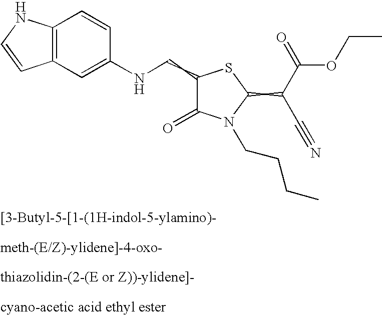 | (DMSO-d6, Stored with K₂CO₃, Main Isomer): δ =0.92 (t, 3H); 1.27 (t, 3H); 1.33 (m, 2H); 1.61 (m,2H); 4.10-4.3 1 (m, 4H); 6.41 (s, 1H); 7.06 (d, 1H); 7.32-7.42 (m, 2H); 7.45 (s, 1H); 8.19 (s, 1H); 10.61 (s, 1H); 11.13 (s, 1H) ppm. | MW: 410.50 MS (ESI) [M + 1]⁺: 411 | INT130/ INT132 |
| 93 | 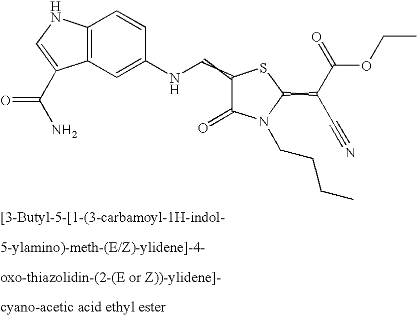 | (DMSO-d6, Stored K₂CO₃, Main Isomer): δ =0.91 (t, 3H); 1.27 (t, 3H); 1.34 (m, 2H); 1.61 (m, 2H); 4.10-4.30 (m, 4H); 6.70-7.22 (m, 2H); 7.32-7.50 (m, 2H); 7.95-8.09 (m, 2H); 8.23 (s, 1H); 10.77 (s, 1H); 11.58 (s, 1H) ppm. | MW: 453.52 MS (ESI) [M + 1]⁺: 454 | INT130/ INT132 |
| 94 | 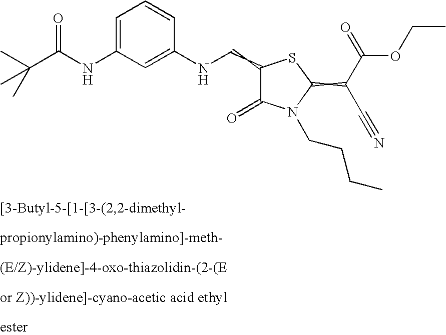 | (DMSO-d6, Stored with K₂CO₃, Main Isomer): δ =0.90 (t, 3H); 1.24 (s, 9H); 1.26 (t, 3H); 1.33 (m, 2H); 1.62 (m, 2H); 4.13-4.28 (m, 4H); 6.94 (d, 1H); 7.26 (t, 1H); 7.38 (d, 1H); 7.73 (s, 1H); 8.12 (s, 1H); 9.26 (s, 1H); 10.63 (s, 1H) ppm. | MW: 470.59 MS (ESI) [M + 1]⁺: 471 | INT130/ INT132 |
| 95 | 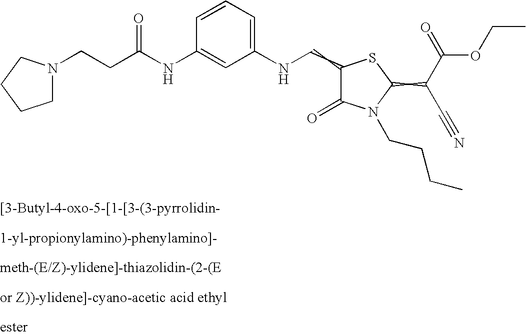 | (DMSO-d6, Stored with K₂CO₃, Main Isomer): δ =0.91 (t, 3H); 1.26 (t, 3H); 1.33 (m, 2H); 1.61 (m, 2H); 1.69 (m, 4H); 2.49-2.57 (m, 6H); 2.72 (t, 2H); 4.11-4.29 (m, 4H); 6.93 (s, 1H); 7.13-7.30 (m, 2H); 7.68 (s, 1H); 8.15 (s, 1H); 10.12 (s, 1H); 10.67 (s, 1H) ppm. | MW: 511.64 MS (ESI) [M + 1]⁺: 512 | INT130/ INT132 |
| 96 | 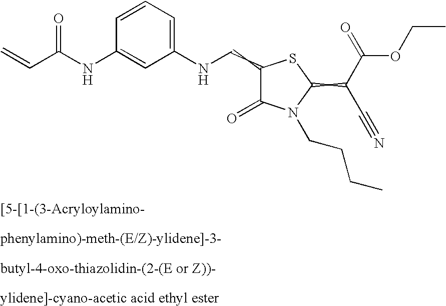 | (DMSO-d6, Stored with K₂CO₃, Main Isomer): δ =0.91 (t, 3H); 1.27 (t, 3H); 1.33 (m, 2H); 1.61 (m, 2H); 4.11-4.29 (m, 4H); 5.78 (dd, 1H); 6.28 (dd, 1H); 6.44 (dd, 1H); 6.99 (m, 1H); 7.22-7.31 (m, 2H); 7.75 (s, 1H); 8.14 (s, 1H); 10.20 (s, 1H); 10.68 (s, 1H) ppm. | MW: 440.52 MS (ESI) [M + 1]⁺: 441 | INT130/ INT132 |
| 97 | 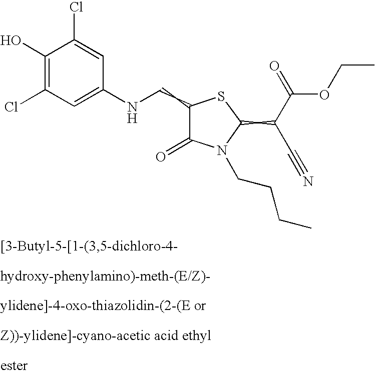 | (DMSO-d6, Stored with K₂CO₃, Main Isomer): δ =0.91 (t, 3H); 1.27 (t, 3H); 1.33 (m, 2H); 1.61 (m, 2H); 4.10-4.30 (m, 4H); 7.37 (s, 2H); 8.15 (s, 1H); 9.50-10.70 (b, 2H) ppm. | MW: 456.35 MS (ESI) [M + 1]⁺: 457 | INT130/ INT132 |
| 98 | 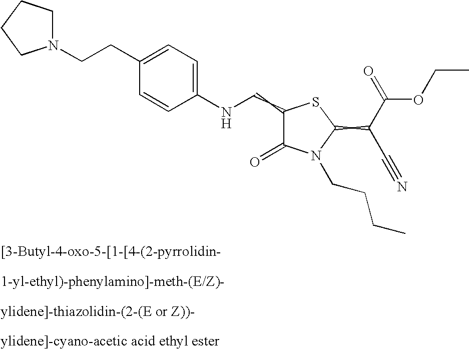 | (DMSO-d6, Stored with K₂CO₃, Main Isomer): δ =0.91 (t, 3H); 1.26 (t, 3H); 1.32 (m, 2H); 1.53-1.72 (m, 6H); 2.46 (m, 4H); 2.59 (m, 2H); 2.70 (m, 2H); 4.12-4.29 (m, 4H); 7.19 (m, 4H); 8.19 (s, 1H); 10.52 (s, 1H) ppm. | MW: 468.62 MS (ESI) [M + 1]⁺: 469 | INT130/ INT132 |
| 99 | 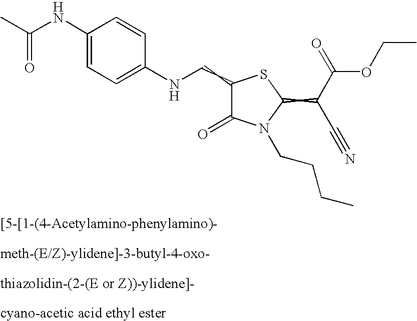 | (DMSO-d6, Stored with K₂CO₃, Main Isomer): δ =0.91 (t, 3H); 1.26 (t, 3H); 1.33 (m, 2H); 1.62 (m, 2H); 2.03 (s, 3H); 4.12-4.28 (m, 4H); 7.23 (d, 2H); 7.55 (d, 2H); 8.15 (s, 1H); 9.94 (s, 1H); 10.54 (s, 1H) ppm. | MW: 428.51 MS (ESI) [M + 1]⁺: 429 | INT130/ INT132 |
| 100 | 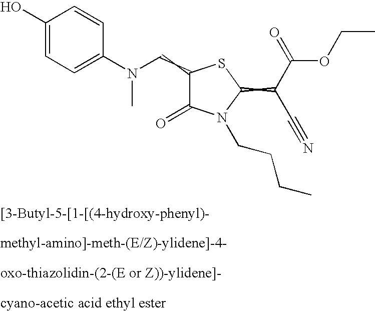 | (DMSO-d6, Stored with K₂CO₃, Main Isomer): δ =0.89 (t, 3H); 1.18 (t, 3H); 1.29 (m, 2H); 1.55 (m, 2H); 3.53 (s, 3H); 4.00-4.22 (m, 4H); 6.86 (d, 2H); 7.21 (d, 2H); 7.98 (s, 1H); 9.92 (s, 1H) ppm. | MW: 401.49 MS (ESI) [M + 1]⁺: 402 | INT130/ INT132 |
| 101 | 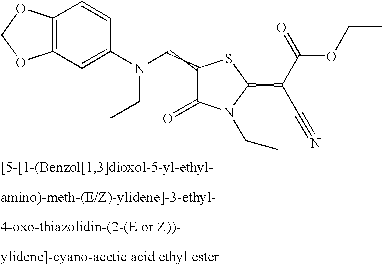 | (DMSO-d6, Stored with K₂CO₃, Main Isomer): δ =1.03-1.21 (m, 9H); 3.82 (q, 2H); 4.10 (q, 2H); 4.18 (q, 2H); 6.12 (s, 2H); 6.83 (dd, 1H); 7.01-7.10 (m, 2H); 8.00 (s, 1H) ppm. | MW: 415.47 MS (ESI) [M + 1]⁺: 416 | INT124/ INT131 |
| 102 | 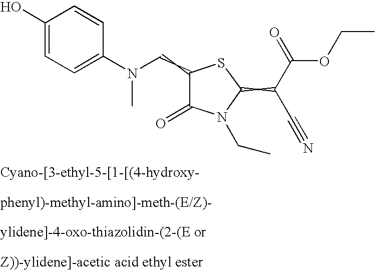 | (DMSO-d6, Stored with K₂CO₃, Main Isomer): δ =1.10-1.23 (m, 6H); 3.53 (s, 3H); 4.09 (q, 2H); 4.20 (q, 2H); 6.87 (d, 2H); 7.20 (d, 2H); 7.99 (s, 1H); 9.99 (s, 1H) ppm. | MW: 373.43 MS (ESI) [M + 1]⁺: 374 | INT124/ INT131 |
| 103 | 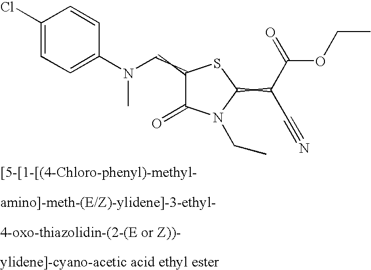 | (CDCl₃, Stored with K₂CO₃, Main Isomer): δ =1.30-1.47 (m, 6H); 3.68 (s, 3H); 4.30 (q, 2H); 4.43 (q, 2H); 7.17 (d, 2H); 7.43 (d, 2H); 7.91 (s, 1H) ppm. | MW: 391.88 MS (ESI) [M + 1]⁺: 392 | INT124/ INT132 |

| | | | Molecu- | |
|---|---|---|---|---|
| | | | lar | Educt/ |
| | | | Weight/ | Syn- |
| | | | MS | thesis as |
| Exam- | | | (ESI) | in the |
| ple No. | Structure and Name | ¹H—NMR | [M + 1]⁺ | Case of |
| 104 | 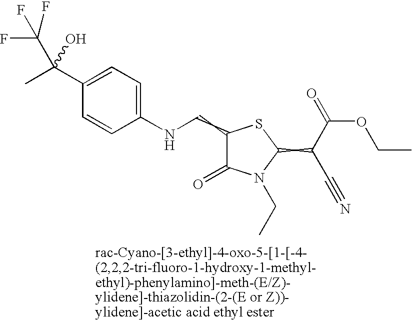 | (DMSO-d6; Main Isomer): δ =1.23-1.28 (m, 6H); 1.67 (s, 3H); 4.20-4.27 (m, 4H); 6.59 (s, 1H); 7.40 (dd, 2H); 8.21 (d, 1H); 10.59 (d, 1H) ppm. | 455.459/ 456 | INT124/ INT132 |
| 105 | 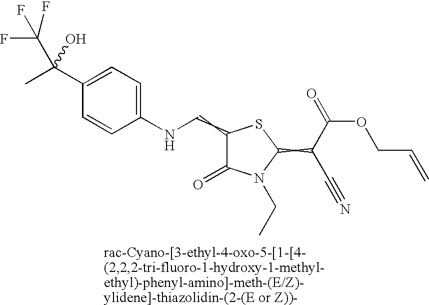 | (DMSO-d6; Main Isomer): δ =1.24 (t, 3H); 1.66 (s, 3H); 4.26 (q, 2H); 4.70 (tt, 2H); 5.25 (qq, 1H); 5.37 (qq, 1H) 7.40 (dd, 2H); 5.96 (in, 1H); 6.56 (s, 1H); 7.31-7.54 (q, 4H) 8.20 (1H); 10.56 (1H) ppm. | 467.470/ 468 | INT126/ INT132 |
| 106 | 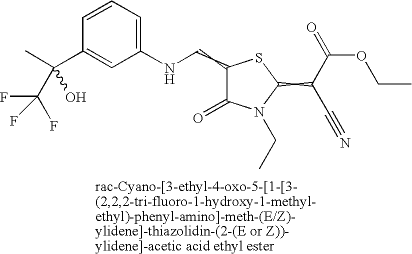 | (DMSO-d6; Main Isomer): δ =1.22-1.28 (m, 6H); 1.69 (s, 3H); 4.19-4.28 (m, 4H); 6.68 (s, 1H); 7.25-7.38 (m, 3H); 7.52 (s, 1H); 8.19 (1H); 10.59 (s, 1H) ppm. | 455.459/ 456 | INT124/ INT132 |
| 107 | 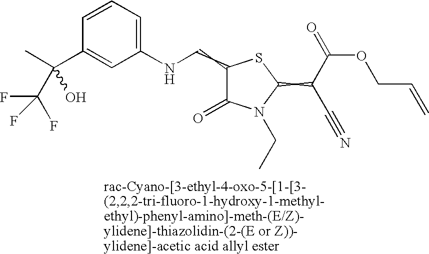 | (DMSO-d6; Main Isomer, Selection): δ =1.21-1.28 (m, 3H); 1.69 (s, 3H); 4.24 (q, 2H); 6.69 (s, 1H); 7.26-7.39 (m, 3H); 7.53 (s, 1H); 8.22 (d, 1H); 10.63 (d, 1H) ppm. | 467.470/ 468 | INT126/ INT132 |
| 108 | 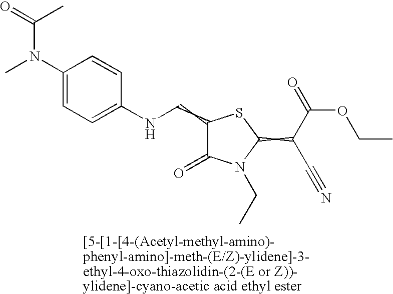 | (DMSO-d6; Main Isomer): δ =1.22-1.28 (m, 6H); 1.76 (s, 3H); 3.11 (s, 3H); 4.21-4.25 (m, 4H); 7.28-7.38 (dd, 4H) 8.19 (s, 1H); 10.55 (s, 1H) ppm. | 414.486/ 415 | INT124/ INT132 |
| 109 | 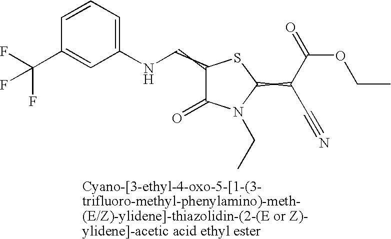 | (DMSO-d6; Main Isomer): δ =1.22-1.28 (m, 6H); 4.19-4.25 (m, 4H); 7.36 (d, 1H); 7.53 (t, 1H); 7.59-7.63 (m, 1H); 8.26 (s, 1H); 10.56 (s, 1H) ppm. | 411.405/ 412 | INT124/ INT132 |
| 110 | 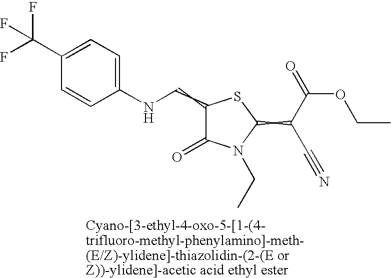 | (DMSO-d6; Main Isomer): δ =1.23-1.28 (2t, 6H); 4.21-4.25 (m,4H); 7.46-7.66 (q, 4 h); 8.22 (s, 1H); 10.68 (s, 1H) ppm. | 411.405/ 412 | INT124/ INT132 |
| 111 | 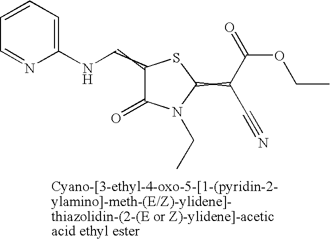 | (DMSO-d6; Main Isomer): δ =1.22-1.28 (m, 6H); 4.18-4.23 (m, 4H); 7.04-7.07 (m, 2H); 7.71-7.76 (m, 1H); 8.28-8.29 (m, 1H); 8.73 (d, 1H); 10.93 (d, 1H) ppm. | 344.394/ 345 | INT124/ INT132 |
| 112 | 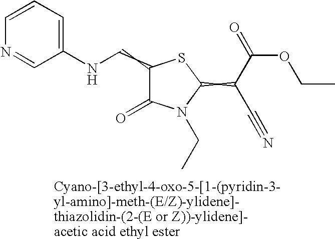 | (DMSO-d6; Main Isomer): δ =1.22-1.28 (m, 6H); 4.19-4.24 (m, 4H); 7.32-7.37 (dd, H); 7.73-7.75 (m, 1H); 8.20 (s, 1H); 8.24-8.25 (m, 1H); 8.53 (d, 1H); 10.52(s, 1H) ppm. | 344.394/ 345 | INT124/ INT132 |
| 113 | 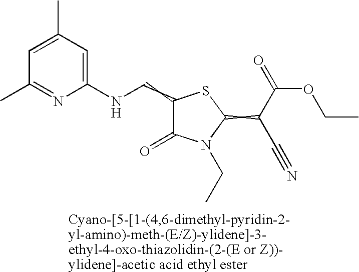 | (DMSO-d6; Main Isomer): δ =1.22-1.28 (m, 6H); 2.24 (s, 3H); 2.38 (s, 3H); 4.18-4.24 (m, 4H); 6.67 (s, 1H); 6.77 (s, 1H), 8.73 (m, 1H); 10.82 (s, lH) ppm. | 372.449 | INT124/ INT132 |
| 114 | 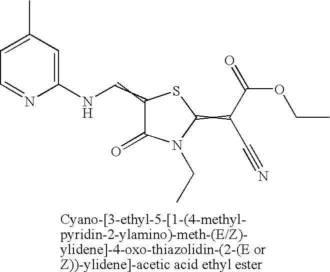 | (DMSO-d6; Main Isomer): δ =1.23-1.26 (2t, 6H); 2.29 (s, 3H); 4.18-4.24 (2q, 4H); 6.65 (d, 1H); 6.89-6.91 (dd, 1H); 8.14(d, 1H); 8.73 (s, 1H); 10.86 (3, H) ppm. | 358.422 | INT124/ INT132 |
| 115 | 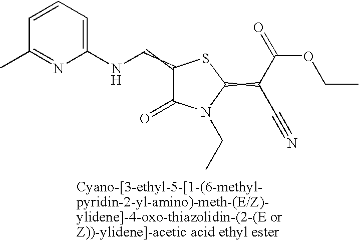 | (DMSO-d6; Main Isomer): δ =1.23-1.26 (2t, 6H); 2.41 (s, 3H); 4.17-4.22 (2q, 4H); 6.83 (d, 1H); 6.89 (d, 1H); 7.59 (t, 1H); 8.71 (1H); 10.86 (s, 1H) ppm: | 358.422 | INT124/ INT132 |
| 116 | 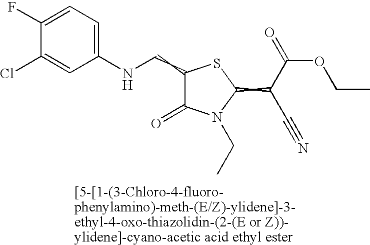 | (DMSO-d6; Main Isomer): δ =1.22-1.27 (m, 6H); 4.21-4.24 (m. 4H); 7.32 (m, 1H); 7.37 (m, 1H); 7.58-7.60 (m, 1H); 8.18 (s, 1H); 10.45 (s, 1H) ppm. | 395.842 | INT124/ INT132 |
| 117 | 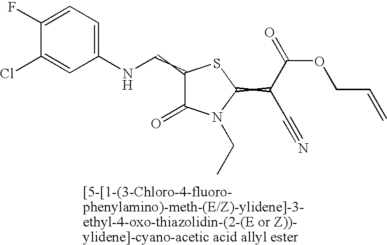 | (DMSO-d6; Main Isomer, Selection): δ =1.22-1.25 (m, 6H); 4.21-4.24 (m, 4H); 7.28-7.38 (m, 2H); 7.56-7.58 (m, 1H); 8.16-8.18 (m, 1H); 10.45 (s, 1H) ppm. | 407.854 | INT126/ INT132 |
| 118 | 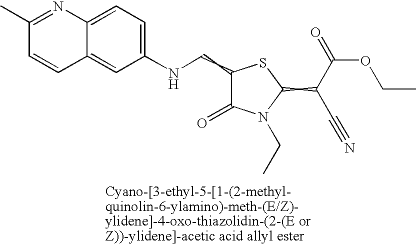 | (DMSO-d6; Main Isomer): δ =1.22-1.28 (2t, 6H); 2.61 (s, 3H); 4.18-4.24 (2q, 4H); 7.33 (d, 1H); 7.63 (dd, 1H); 7.74 (m, 1H); 7.82 (d, 1H); 8.11 (d, 1H); 8.26 (s, 1H); 10.64 (s, 1H) ppm. | 408.482 | INT124/ INT132 |
| 119 | 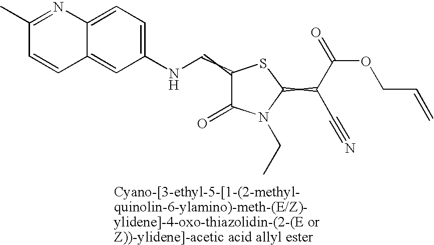 | (DMSO-d6; Main Isomer, Selection): δ =1.25 (t, 3H); 2.60 (s, 3H); 4.22 (q, 2H); 7.33 (d, 1H); 7.61-7.64 (dd, 1H); 7.75 (d, 1H); 7.82 (d, 1H); 8.11 (d,1H); 8.27 (1H); 10.66 (s, 1H) ppm. | 420.493 | INT126/ INT132 |
| 120 | 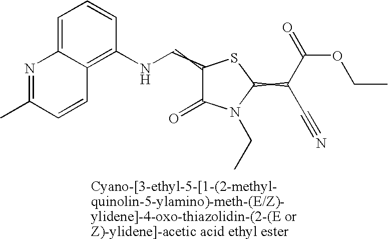 | (DMSO-d6; Main Isomer): δ =1.21-1.26 (m, 6H); 2.66 (s, 3H); 4.17-4.24 (m, 4H); 7.38 (d, 1H); 7.46 (d, 1H); 7.66-7.68 (m, 1H); 7.74 (d, 1H); 8.05 (s, 1H); 8.44 (d, 1H); 10.65 (s, 1H) ppm. | 408.482 | INT124/ INT132 |
| 121 | 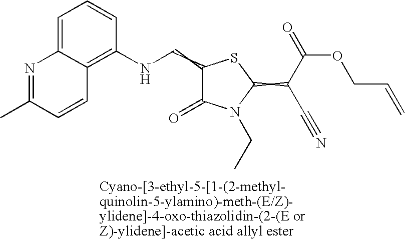 | (DMSO-d6; Main Isomer, Selection): δ =1.24 (t, 3H); 2.66 (s, 3H); 4.22 (q, 2H); 7.40 (d, 2H); 7.47 (d, 1H); 7.66-7.70 (m, 1H); 7.75-7.78 (m, 1H); 8.08 (s, 1H); 8.46 (d, 1H); 10.69 (s, 1H) ppm. | 420.482 | INT126/ INT132 |
| 122 | 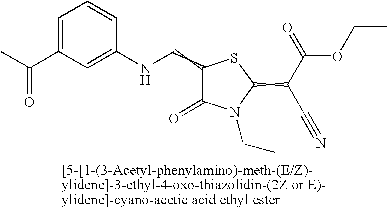 | ¹H—NMR (CDCl₃, 400 MHz) (selected peaks) δ =1.30 (m, 6H); 2.59 (s, 3H); 4.28 (m, 2H); 4.39 (m, 2H); 7.21 (m, 1H); 7.46 (m, 1H); 7.62 (m, 2H); 10.57 (d, 1H). | MW: 385.442 MS (ESI) [M + 1]⁺: 386 | INT124/ INT132 |
| 123 | 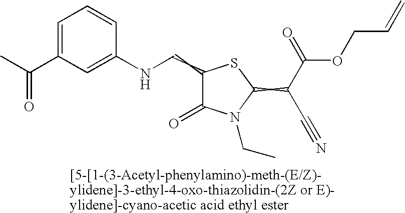 | ¹H—NMR (CDCl₃, 400 MHz) (selected peaks) δ = 1.46 (m, 3H); 2.68 (s, 3H); 4.47 (m, 2H); 4.79 (m, 2H); 5.31 (dd, 1H); 5.42 (d, 1H); 6.02 (m, 1H); 7.32 (m, 1H); 7.53 (m, 1H); 7.74 (m, 2H); 8.25 (d, 1H); 10.70 (d, 1H). | MW: 397.453 MS (ESI) [M + 1]⁺: 398 | INT126 /INT132 |
| 124 | 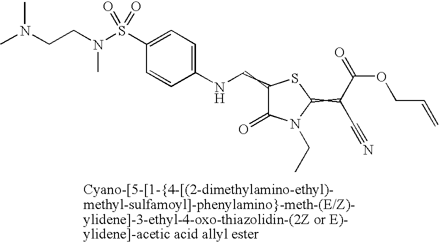 | ¹H—NMR (DMSO-d6, 300 MHz) (selected peaks) δ = 1.22 (m, 3H); 2.19 (s, 6H); 2.42 (m, 2H); 2.71 (s, 3H); 3.03 (m, 2H); 4.28 (m, 2H); 4.72 (d, 2H); 5.28 (dd, 1H); 5.40 (dd, 1H); 6.00 (m, 1H); 7.51 (d, 2H); 7.73 (d, 2H); 8.28 (s, 1H); 10.70 (s, 1H). | MW: 519.644 MS (ESI) [M + 1]⁺: 520 | INT126/ INT132 |
| 125 | 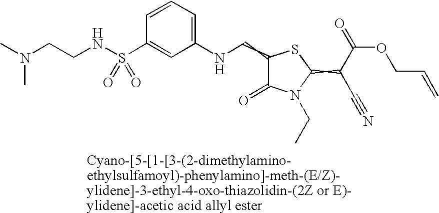 | ¹H—NMR (DMSO-d6, 300 NMHz) (selected peaks) δ = 1.24 (m, 3H); 2.10 (s, 6H); 2.30 (m, 2H); 2.88 (m, 2H); 4.25 (m, 2H); 4.71 (d, 2H); 5.28 (dd, 1H); 5.40 (dd, 1H); 6.00 (m, 1H); 7.49 (dd, 1H); 7.60 (m, 3H); 7.75 (s, 1H); 8.29 (s, 1H); 10.71 (s, 1H). | MW: 505.617 MS (ESI) [M+1]⁺: 506 | INT126/ INT132 |
| 126 | 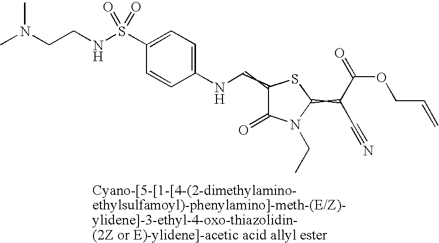 | ¹H—NMR (DMSO-d6, 300 MHz) (selected peaks) δ = 1.22 (m, 3H); 2.10 (s, 6H); 2.29 (m, 2H); 2.80 (m, 2H); 4.26 (m, 2H); 4.71 (d, 2H); 5.29 (dd, 1H); 6.00 (m, 1H); 7.48 (s, 1H); 7.49 (d, 2H); 7.74 (d, 2H); 8.30 (s, 1H); 10.70 (s, 1H). | MW: 505.617 MS (ESI) [M + 1]⁺: 506 | INT126/ INT132 |
| 127 | 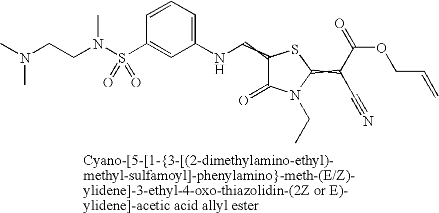 | ¹H—NMR (DMSO-d6, 300 MHz) (selected peaks) δ = 1.24 (m, 3H); 2.19 (s, 6H); 2.42 (m, 2H); 2.72 (s, 3H); 3.09 (m, 2H); 4.27 (m, 2H); 4.72 (d, 2H); 5.28 (dd, 1H); 5.39 (dd, 1H); 6.00 (m, 1H); 7.45 (d, 1H); 7.61 (m, 1H); 7.69 (m, 2H); 8.31 (s, 1H); 10.62 (s, 1H). | MW: 519.644 MS (ESI) [M + 1]⁺: 520 | INT126/ INT132 |
| 128 | 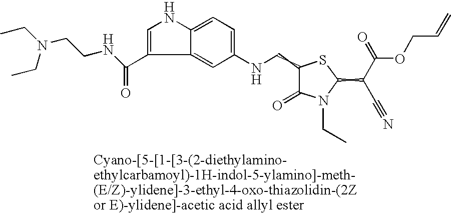 | ¹H—NMR (DMSO-d6, 300 MHz) δ =0.97 (m, 6H); 1.26 (m, 3H); 4.25 (m, 2H); 4.71 (d, 2H); 5.28 (dd, 1H); 5.38 (dd, 1H); 6.0 (m, 1H); 7.27 (dd, 1H); 7.42 (d, 1H); 7.38 (m, 1H); 8.0 (d, 1H); 8.07 (d, 1H); 8.21 (s, 1H); 10.77 (s, 1H); 11.59 (s, 1H). | MW: 536.654 MS (ESI) [M+1]⁺: 537 | INT126/ INT132 |
| 129 | 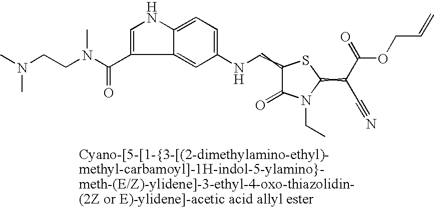 | ¹H—NMR (DMSO-d6, 300 MHz) (selected peaks) δ =1.28 (m, 3H); 2.15 (s, 6H); 3.11 (s, 3H); 3.59 (m, 2H); 4.26 (m, 2H); 4.72 (d, 2H); 3.27 (dd, 1H); 3.39 (dd, 1H); 6.0 (m, 1H); 7.19 (dd, 1H); 7.42 (d, 1H); 1H); 7.69 (m, (d, 1H); 8.18 (s, 1H); 10.70 (s, 1H); 11.60 (s, 1H). | MW: 522.627 MS (ESI) [M + 1]⁺: 523 | INT126/ INT132 |
| 130 | 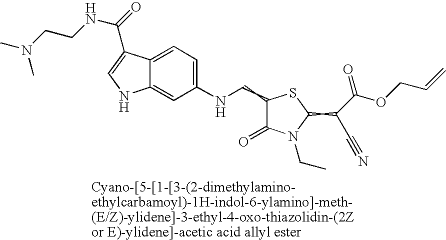 | ¹H—NMR (DMS0-d6, 300 MHz) (selected peaks) δ =1.26 (m, 3H); (m, 2H); 4.28 (m, 2H); 4.70 (d, 2H); 5.28 (dd, 1H); 5.40 (dd, 1H); 6.0 (m, 1H); 7.11 (dd, 1H); 7.35 (s, 1H); 7.80 (m, 1H); 7.98 (d, 1H); 8.08 (d, 1H); 8.25 (s, 1H); 10.63 (s, 1H); 11.50 (s, 1H). | MW: 508.600 MS (ESI) [M + 1]⁺: 509 | INT126/ INT132 |
| 131 | 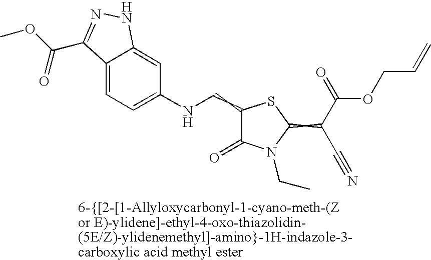 | ¹H—NMR (DMSO-d6, 300 MHz) (selected peaks) δ =1.28 (m, 3H); 3.91 (s, 3H); 4.22 (m, 2H); 4.71 (d, 2H); 5.29 (dd, 1H); 5.40 (dd, 1H); 5.97 (m, 1H); 7.32 (dd, 1H); 7.50 (s, 1H); 8.00 (d, 1H); 8.30 (s, 1H); 10.73 (s, 1H). | MW: 453.477 MS (ESI) [M + 1]⁺: 454 | INT126/ INT132 |
| 132 | 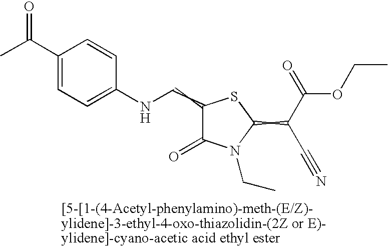 | ¹H—NMR (CDCl₃, 400 MHz) (selected peaks) δ =1.30 (m, 6H); 2.55 (s, 3H); 4.25 (m, 2H); 4.38 (m, 2H); 7.05 (d, 2H); 7.58 (d, 1H); 7.95 (d, 2H); 10.60 (d, 1H). | MW: 385.442 MS (ESI) [M +1]⁺: 386 | INT124/ INT132 |
| 133 | 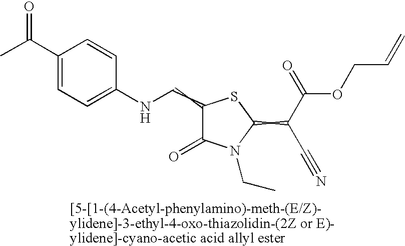 | ¹H—NMR (CDCl₃, 400 MHz) (selected peaks) δ =1.32 (m, 3H); 2.52 (s, 3H); 4.38 (m, 2H); 4.70 (m, 2H); 5.22 (dd, 1H); 5.36 (dd, 1H); 5.90 (m, 1H); 7.08 (d, 2H); 7.60 (d, 1H); 7.92 (d, 2H); 10.61 (d, 1H). | MW: 397.453 MS (ESI) [M + 1]⁺: 340 | INT126/ INT132 |
| 134 | 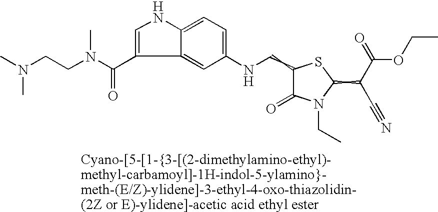 | ¹H—NMR (DMSO-d6, 300 MHz) (selected peaks) δ =1.26 (m, 6H); 2.18 (s, 6H); 3.11 (s, 3H); 3.49 (m, 2H); 4.25 (m, 4H); 7.20 (dd, 1H); 7.42 (d, 1H); 7.71 (s, 1H); 7.78 (d, 1H); 8.16 (s, 1H); 10.70 (s, 1H); 11.60 (s, 1H). | MW: 510.616 MS (ESI) [M + 1]⁺: 511 | INT124/ INT132 |
| 135 | 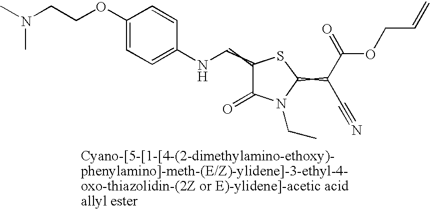 | ¹H—NMR (DMSO-d6, 300 MHz) (selected peaks) δ =1.23 (m, 3H); 2.21 (s, 6H); 2.62 (m, 2H); 4.03 (m, 2H); 4.23 (m, 2H); 4.71 (d, 2H); 5.27 (dd, 1H); 5.39 (dd, 1H); 5.98 (m, 1H); 6.95 (d, 2H); 7.26 (d, 2H); 8.12 (s, 1H); 10.50 (s, 1H). | MW: 442.537 MS (ESI) [M + 1]⁺: 443 | INT126/ INT132 |
| 136 | 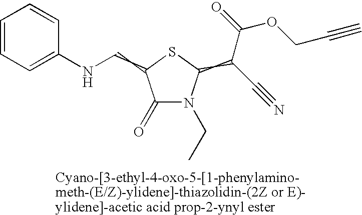 | ¹H—NMR (CDCl₃, 300 MHz) (selected peaks) δ = 1.42 (m, 3H); 2.51 (m, 1H); 4.45 (m, 2H); 4.88 (d, 2H); 7.09 (m, 2H); 7.20 (m, 1H); 7.40 (m, 2H); 7.66 (d, 1H); 10.61 (d, 1H). | MW: 353.40 MS (ESI) [M + 1]⁺: 354 | INT138 |
| 137 | 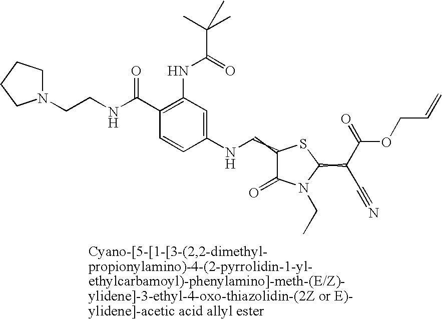 | ¹H—NMR (CDCl₃, 300 (MHz) (selected peaks) δ =1.32 (m, 9H); 1.41 (m,9H); 1.80 (m, 4H); 2.53 (m, 4H); 2.71 (m, 2H); 3.49 (m, 2H); 4.40 (m, 2H); 4.72 (m, 2H); 5.25 (dd, 1H); 5.38 (dd, 1H); 5.95 (m, 1H); 6.69 (dd, 1H); 7.02 (m, 1H); 7.50 (d, 1H); 7.70 (s, 1H); 8.70 (s, 1H); 10.60 (s, 1H); 11.97 (s, 1H). | MW: 594.733 MS (ESI) [M + 1]⁺: 595 | INT126/ INT132 |
| 138 | 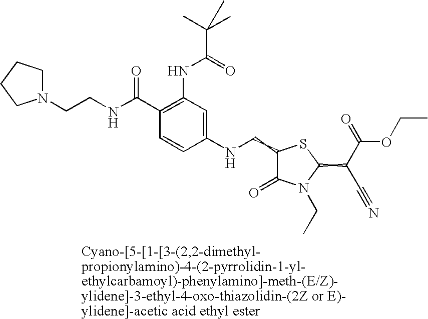 | ¹H—NMR (CDCl₃, 300 MHz) (selected peaks) δ =1.36 (m, 15H); 1.79 (m, 4H); 2.56 (m, 4H); 2.71 (m, 2H); 3.50 (m, 2H); 4.29 (m, 2H); 4.39 (m, 2H); 6.68 (dd, 1H); 7.06 (m, 1H); 7.48 (d, 1H); 7.68 (s, 1H); 8.70 (d, 1H); 10.56 (s, 1H); 11.97 (s, 1H). | MW: 582.722 MS (ESI) [M + 1]⁺: 583 | INT124/ INT132 |
| 139 | 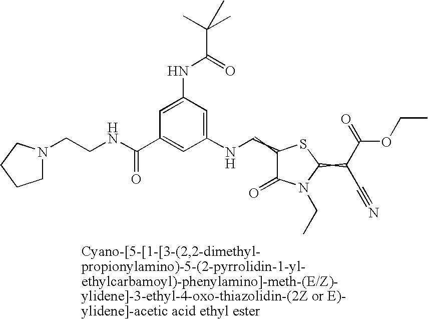 | ¹H—NMR (DMSO-d6, 300 MHz) (selected peaks) δ =1.25 (m, 15H); 1.70 (m, 4H); 2.60 (m, 2H); 3.39 (m, 2H); 4.26 (m, 4H); 7.44 (s, 1H); 7.74 (s, 1H); 7.98 (s, 1H); 8.28 (s, 1H); 8.52 (m, 1H); 9.42 (s, 1H); 10.71 (s, 1H). | MW: 582.722 MS (ESI) [M + 1]⁺: 583 | INT124/ INT132 |
| 140 | 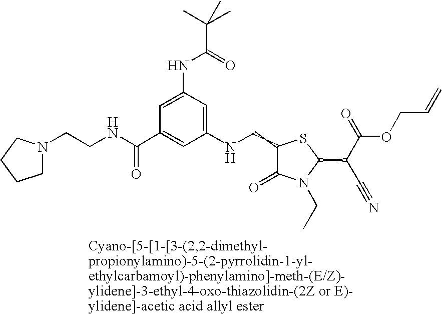 | ¹H—NMR (DMSO-d6, 300 MHz) (selected peaks) δ =1.22 (m, 15H); 1.70 (m, 4H); 2.61 (m, 2H); 3.40 (m, 2H); 4.28 (m, 2H); 4.71 (d, 2H); 5.27 (dd, 1H); 5.39 (dd, 1H); 6.00 (m, 1H); 7.42 (s, 1H); 7.77 (s, 1H); 7.97 (s, 1H); 8.28 (s, 1H); 8.52 (m, 1H); 9.42 (s, 1H); 10.76 (s, 1H). | MW: 594.733 MS (ESI) [M + 1]⁺: 595 | INT126/ INT132 |

### EXAMPLE 141

### [5-[1-[Acetyl-(6-amino-pyridin-3-yl)-amino]-meth-(E/Z)-ylidene]-3-ethyl-4-oxo-thiazolidin-(2-(E or Z))-ylidene]-cyano-acetic acid ethyl ester

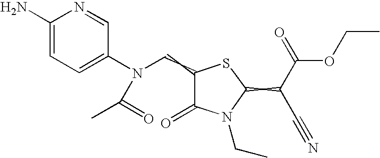

420 mg of the compound that is described under Example 82) and 0.13 ml of triethylamine are dissolved in 5 ml of dichloromethane. 0.02 ml of acetic anhydride is added, and it is stirred for 2 hours at room temperature. The reaction mixture is mixed with dichloromethane and washed three times with semi-saturated sodium bicarbonate solution. The organic phase is dried on sodium sulfate. After purification by chromatography on silica gel, 340 mg of the title compound is obtained.

(DMSO-d6, stored with K2CO3, main isomer): δ=1.12-1.28 (t, 3H); 2.01 (s, 3H); 4.09-4.27 (m, 4H); 6.51-6.64 (m, 3H); 7.46 (dd, 1H); 7.98 (d, 1H); 8.55 (s, 1H) ppm.

The examples below describe the production of compounds according to the invention without the latter being limited to these examples. These compounds can also be used as intermediate substances in the production of substances of general formula (I) according to the invention.

### EXAMPLE 142

### (E or Z)-Cyano-(3-ethyl-4-oxo-5-(E/Z)-{[4-(2-pyrrolidin-1-yl-ethyl)-phenylamino]-methylene}-thiazolidin-2-ylidene)-acetic acid

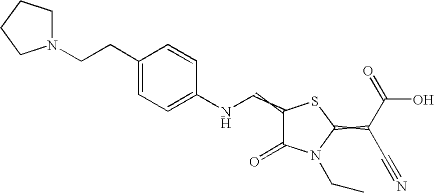

2.05 g of potassium-(tert)-butylate is introduced into 50 ml of tetrahydrofuran at 0° C. and mixed with 76.4 μl of water. 1.0 g of the compound that is described under Example INT131) is added and stirred for 30 minutes at 0° C., and for 20 hours at room temperature. At 0° C., 8.25 ml of 2-molar hydrochloric acid in diethyl ether is added, and it is stirred for one hour at room temperature. The solvent is condensed under high vacuum, and the residue is further reacted without additional purification.

Molar mass=412.514; MS (ESI): [M+1]+=413.

### EXAMPLE 143

### (E or Z)-Cyano-(3-ethyl-5-(E/Z-({4-[3-(4-methyl-piperazin-1-yl)-propionylamino]-phenylamino}-methylene}-4-oxo-thiazolidin-2-ylidene)-acetic acid

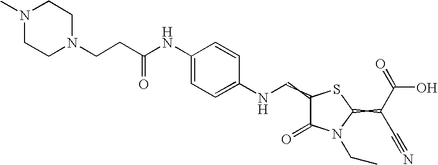

4.4 g of the compound that is described under Example 3), 0.91 g of Pd(PPh₃)₄ and 6.9 ml of morpholine are stirred in 150 ml of tetrahydrofuran for 15 minutes. After 45 ml of triethylamine is added, the reaction mixture that is obtained is evaporated to the dry state in a rotary evaporator. The thus obtained crude product is purified by chromatography with a dichloromethane/methanol mixture on silica gel. 3.5 g of the title compound is obtained as a pH-dependent 5-(E/Z)-isomer mixture.

1H-NMR (DMSO-d6, main isomer): δ=1.20 (3H); 2.19 (3H); 2.23-2.55 (10H) 2.61 (2H); 4.20 (2H); 7.18 (2H); 7.52 (2H); 7.87 (1H); ppm.

The compounds below are produced analogously to the above-described process.

| | | | | | Syn- |
|---|---|---|---|---|---|
| | | | | | thesis as |
| Example | | Molecular | MS (ESI) | | in the |
| No. | Structure | Weight | [M + 1]⁺ | Educt | Case of |
| 144 | 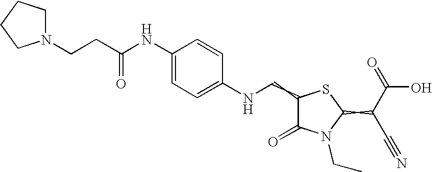 Cyano-[3-ethyl-4-oxo-5-[1-[4-(3- pyrrolidin-1-yl-propionylamino)- phenylamino]-meth-(E/Z)-ylidene]- thiazolidin-(2-(E or Z))-ylidene]-acetic acid | 455.54 | 456 | 46 | 143 |
| 145 | 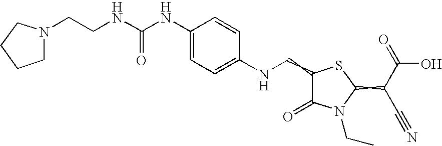 Cyano-[3-ethyl-4-oxo-5-[1-{4-[3-(2-pyrrolidin-1-yl-ethyl)-ureido]- phenylamino}-meth-(E/Z)-ylidene]- thiazolidin-(2-(E or Z))-ylidene]-acetic acid | 470.55 | 471 | 47 | 143 |
| 146 | 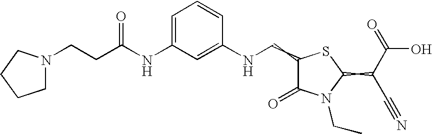 Cyano-[3-ethyl-4-oxo-5-[1-[3-(3- pyrrolidin-1-yl-propionylamino)- phenylamino]-meth-(E/Z)-ylidene]- thiazolidin-(2-(E or Z))-ylidene]-acetic acid | 455.54 | 456 | 49 | 143 |
| 147 | 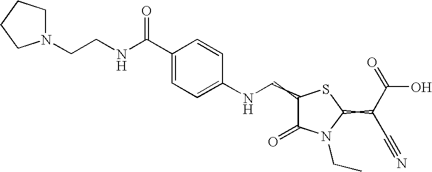 Cyano-[3-ethyl-4-oxo-5-[1-[4-(2- pyrrolidin-1-yl-ethylcarbamoyl)- phenylamino]-meth-(E/Z)-ylidene]- thiazolidin-(2-(E or Z))-ylidene]-acetic acid | 455.54 | 456 | 5 | 143 |
| 148 | 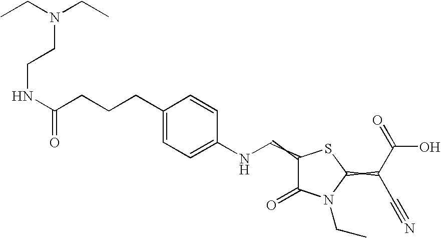 Cyano-[5-[1-{4-[3-(2-diethylamino- ethylcarbamoyl)-propyl]- phenylamino}-meth-(E/Z)-ylidene]-3- ethyl-4-oxo-thiazolidin-(2-(E or Z))- ylidene]-acetic acid | 499.637 | 500 | 52 | 143 |
| 149 | 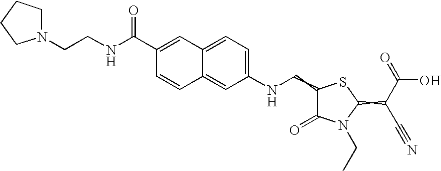 Cyano-[3-ethyl-4-oxo-5-[1-[6-(2- pyrrolidin-1-yl-ethylcarbamoyl)- naphthalen-2-ylamino]-meth-(E/Z)- ylidene]-thiazolidin-(2-(E or Z))- ylidene]-acetic acid | 505.60 | 506 | 53 | 143 |
| 150 | 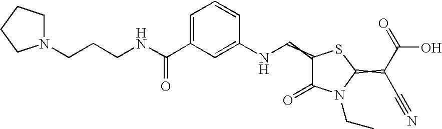 Cyano-[3-ethyl-4-oxo-5-]1-[3-(3- pyrrolidin-1-yl-propylcarbamoyl)- phenylamino]-meth-(E/Z)-ylidene]- thiazolidin-(2-(E or Z))-ylidene]-acetic acid | 469.57 | 470 | INT13 4 | 142 |
| 151 | 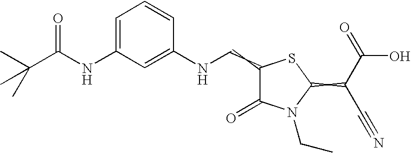 Cyano-[5-[1-[3-(2,2-dimethyl- propionylamino)-phenylamino]-meth- (E/Z)-ylidene]-3-ethyl-4-oxo- thiazolidin-(2-(E or Z))-ylidene]-acetic acid | 414.49 | 415 | INT13 5 | 142 |
| 152 | 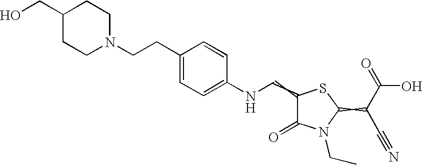 Cyano-[3-ethyl-5-[1-{4-[2-(4- hydroxymethyl-piperidin-1-yl)-ethyl]- phenylamino}-meth-(E/Z)-ylidene]-4- oxo-thiazolidin-(2-(E or Z))-ylidene]- acetic acid | 456.57 | 457 | INT13 - 6 | 142 |
| 153 | 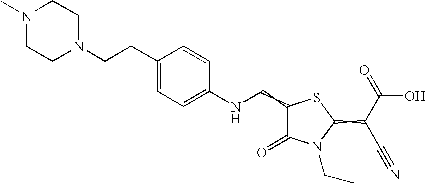 Cyano-[3-ethyl-5-[1-{4-[2-(4-methyl- piperazin-1-yl)-ethyl]-phenylamino}- meth-(E/Z)-ylidene9 -4-oxo-thiazolidin- (2-(E or Z))-ylidene]-acetic acid | 441.56 | 442 | INT13 7 | 142 |
| 154 | 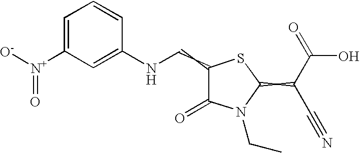 Cyano-[3-ethyl-5-[1-(3-nitro- phenylamino)-meth-(E/Z)-ylidene]-4- oxo-thiazolidin-(2-(E or Z))-ylidene]- acetic acid | 360.351 | 361 | 8 | 142 |

| | | | Molecu- | Educt/ |
|---|---|---|---|---|
| | | | lar | Syn- |
| | | | Weight/ | thesis |
| | | | MS | as in |
| Example | | | (ESI) | the |
| No. | Structure and Name | ¹H-NMR | [M + 1[⁺ | Case of |
| 155 | 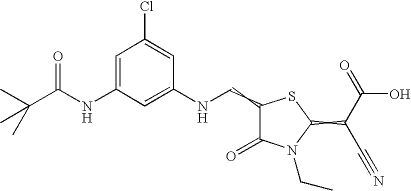 [5-[1-[3-Chloro-5-(2,2-dimethyl- propionylamino)-phenylamino]-meth- (E/Z)-ylidene]-3-ethyl-4-oxo- thiazolidin-(2-(E or Z))-ylidene]-cyano-acetic acid | (DMSO-d6, Stored with K₂CO₃, Main Isomer): δ =1.12-1.29 (m, 12H); 4.21 (q, 2H); 7.00 (s, 1H); 7.52 (s, 1H); 7.61 (s, 1H); 7.89 (s, 1H); 9.37 (s, 1H); 10.18 (s, 1H); 11.5-12.5 (b, 1H) ppm | MW: 448.93 MS (ESI) | |
| # [M + 1]⁺: 449 | 77/143 | | | |

| | | Molecular | Weight/ |
|---|---|---|---|
| Example | | MS (ESI) | Educt/Synthesis |
| No. | Structure and Name | [M + 1]⁺ | As in the Case of |
| 156 | 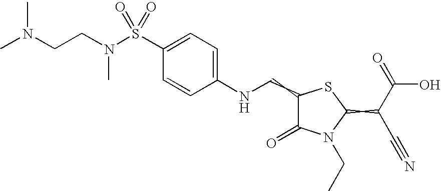 Cyano-[5-[1-{4-[(2-dimethylamino-ethyl)- methyl-sulfamoyl]-phenylamino}-meth-(E/Z)- ylidene]-3-ethyl-4-oxo-thiazolidin-(2Z or E)- yhdene]-acetic acid | MW: 534.569 MS (ESI) [M + 1]⁺: 535 | 124/143 |
| 157 | 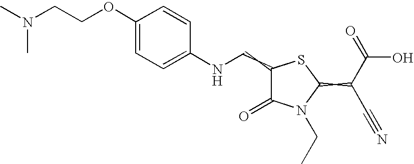 Cyano-[5-[1-[4-(2-dimethylamino-ethoxy)- phenylamino]-meth-(E/Z)-ylidene]-3-ethyl-4- oxo-thiazolidin-(2Z or E)-ylidene]-acetic acid | MW: 402.472 MS (ESI) [M + 1]⁺: 403 | 135/143 |
| 158 | 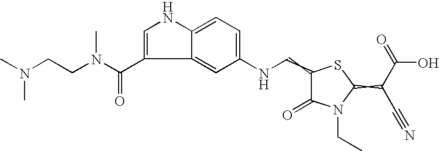 Cyano-[5-[1-{3-[(2-dimethylanilno-ethyl)- methyl-carbamoyl]-1H-indol-5-ylamino}- meth-(E/Z)-ylidene]-3-ethyl-4-oxo-thiazolidin- (2Z or E)-ylidene]-acetic acid | MW: 482.562 MS (ESI) [M + 1]⁺: 483 | 129/143 |
| 159 | 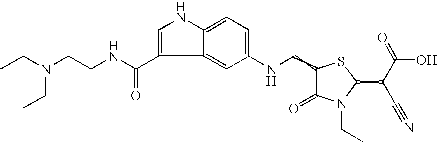 Cyano-[5-[1-[3-(2-diethylamino- ethylcarbamoyl)-1H-indol-5-ylamino]-meth- (E/Z)-ylidene]-3-ethyl-4-oxo-thiazolidin-(2Z or E)-ylidene]-acetic acid | MW: 496.59 MS (ESI) [M + ‘]⁺: 497 | 128/143 |
| 160 | 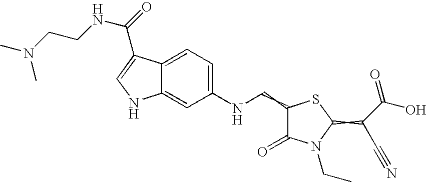 Cyano-[5-[1-[3-(2-dimethylamino- ethylcarbamoyl)-1H-indol-6-ylamino]-meth- (E/Z)-ylidene]-3-ethyl-4-oxo-thiazolidin-(2Z or E)-ylidene]-acetic acid | MW: 468.535 MS (ESI) [M + 1]⁺: 469 | 130/143 |
| 161 | 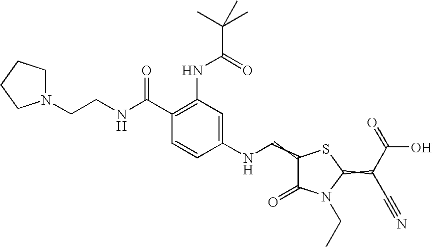 Cyano-[5-[1-[3-(2,2-dimethyl- propionylamino)-4-(2-pyrrolidin-1-yl- ethylcarbamoyl)-phenylamino]-meth-(E/Z)- ylidene]-3-ethyl-4-oxo-thiazolidin-(2-(E or Z))-ylidene]-acetic acid | MW: 554.668 MS (ESI) [M + 1]⁺: 555 | 137/143 |
| 162 | 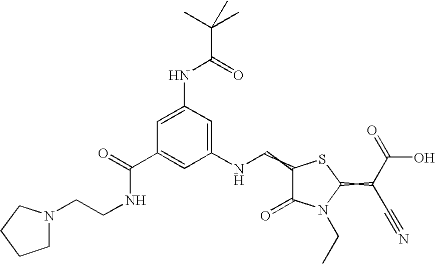 Cyano-[5-[1-[3-(2,2-dimethyl- propionylamino)-5-(2-pyrrolidin-1-yl- ethylcarbamoyl)-phenylamino]-meth-(E/Z)- ylidene]-3-ethyl-4-oxo-thiazolidin-(2-(E or Z))-ylidene]-acetic acid | MW: 554.668 MS (ESI) [M + 1]⁺: 555 | 140/143 |
| 163 | 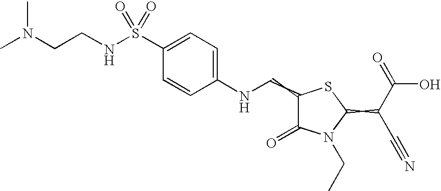 Cyano-[5-[1-[4-(2-dimethylamino- ethylsulfamoyl)-phenylamino]-meth-(E/Z)- ylidene]-3-ethyl-4-oxo-thiazolidin- (2Z or E)-ylidene]-acetic acid | MW: 465.552 MS (ESI) [M + 1]⁺: 466 | 126/143 |
| 164 | 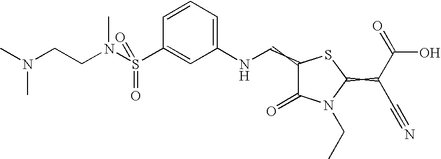 Cyano-[5-[1-{3-[(2-dimethylamino-ethyl)- methyl-sulfamoyl]-phenylamino}- meth-(E/Z)-ylidene]-3-ethyl-4-oxo- thiazolidin-(2Z or E)-ylidene]-acetic acid | MW: 534.659 MS (ESI) [M + 1]⁺: 535 | 127/143 |
| 165 | 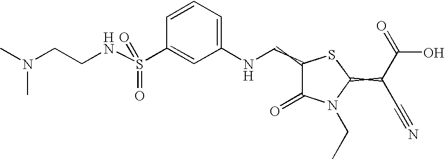 Cyano-[5-[1-[3-(2-dimethylamino- ethylsulfamoyl)-phenylamino]-meth-(E/Z)- ylidene]-3-ethyl-4-oxo-thiazolidin- (2Z or E)-ylidene]-acetic acid | MW: 520.632 MS (ESI) [M + 1]⁺: 521 | 125/143 |

The examples below describe the production of the compounds of general formula (I) according to the invention, without the latter being limited to these examples.

### EXAMPLE 166

### 2-(E or Z)-Cyano-N-ethyl-2-(3-ethyl-4-oxo-5-(E/Z)-{[4-(2-pyrrolidin-1-yl-ethyl)-phenylamino]-methylene}-thiazolidin-2-ylidene)-acetamide

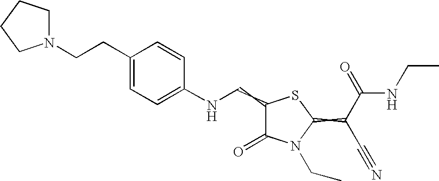

275 mg of the crude product that is described under Example 142) (about 0.2 mmol) is dissolved in 10 ml of dimethylformamide, mixed with 139 μl of triethylamine, 150 μl of a 2M solution of ethylamine in tetrahydrofuran and 96 mg of TBTU and stirred for 20 hours at room temperature. The reaction mixture is mixed with semi-saturated sodium bicarbonate solution and extracted with dichloromethane. The organic solution is washed with saturated sodium chloride solution, dried on sodium sulfate, concentrated by evaporation, and after purification by chromatography on silica gel, 51 mg of the title compound is obtained as a pH-dependent 5-(E/Z)-isomer mixture.

1H-NMR (DMSO-d6, stored with K2CO3, main isomer): δ=1.07 (t, 3H); 1.23 (t, 3H); 1.65 (m, 4H); 2.45 (m, 4H); 2.54-2.62 (m, 2H); 2.62-2.75 (m, 2H); 3.20 (pentuplet, 2H); 4.21 (q, 2H); 7.20 (s, 4H); 7.67 (t, 1H); 8.04 (s, 1H); 10.23 (s, 1H) ppm.

### EXAMPLE 167

### 2-(E or Z)-{5-(E/Z)-[(3-Amino-phenylamino)-methylene]-3-ethyl-4-oxo-thiazolidin-2-ylidene}-2-cyano-N-ethyl-acetamide

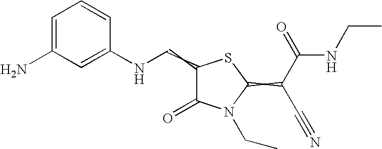

100 mg of the compound that is described under Example 215) is dissolved in 20 ml of ethanol, mixed with 291 mg of tin(II) chloride dihydrate and stirred under reflux for 4 hours. Another 145 mg of tin(II) chloride dihydrate is added, and it is stirred under reflux for another 2 hours. The reaction mixture is mixed with saturated sodium bicarbonate solution, stirred for 30 minutes at room temperature, and extracted with a mixture that consists of chloroform, dichloromethane, and methanol (5:5:1). The organic solution is dried on sodium sulfate, concentrated by evaporation, and after purification by chromatography on amino phase silica gel, 50 mg of the title compound is obtained as a pH-dependent 5-(E/Z)-isomer mixture.

1H-NMR (DMSO-d6, stored with K2CO3, main isomer): δ=1.07 (t, 3H); 1.26 (t, 3H); 3.21 (q, 2H); 4.22 (q, 2H); 5.23 (s, 2H); 6.29 (d, 1H); 6.39 (d, 1H); 6.45 (s, 1H); 6.97 (t, 1H); 7.68 (t, 1H); 7.95 (d, 1H); 10.18 (d, 1H) ppm.

### EXAMPLE 168

### 2-(E or Z)-Cyano-N-ethyl-2-[3-ethyl-5-(E/Z)-({3-[2-(2-methoxy-ethoxy)-acetylamino]-phenylamino}-methylene)-4-oxo-thiazolidin-2-ylidene]-acetamide

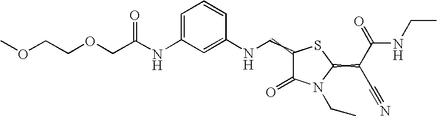

16.5 μl of 2-(2-methoxyethoxy)-acetic acid is introduced into 1 ml of tetrahydrofuran at 0° C. and mixed with 37 μl of triethylamine and 18.5 μl of isobutyl chloroformate. It is stirred for 30 minutes at 0° C., 50 mg of the compound that is described under Example 167), dissolved in 2 ml of tetrahydrofuran, is added, and it is stirred for another 2 hours at room temperature. The reaction mixture is mixed with semi-saturated sodium bicarbonate solution and extracted with dichloromethane. The organic solution is washed with saturated sodium chloride solution, dried on sodium sulfate, concentrated by evaporation, and after purification by chromatography on silica gel, 35 mg of the title compound is obtained as a pH-dependent 5-(E/Z)-isomer mixture.

1H-NMR (DMSO-d6, stored with K2CO3, main isomer): δ=1.08 (t, 3H); 1.25 (t, 3H); 3.12-3.25 (m, 2H); 3.30 (s, 3H); 3.54 (t, 2H); 3.68 (t, 2H); 4.09 (s, 2H); 4.22 (q, 2H); 6.97 (s, 1H); 7.20-7.30 (m, 2H); 7.55-7.77 (m, 2H); 8.04 (s, 1H); 9.68 (s, 1H); 10.39 (s, 1H) ppm.

The examples below are produced analogously to the above-described process.

LENGTHY TABLE REFERENCED HERE
US20070037862A1-20070215-T00001
Please refer to the end of the specification for access instructions.

### EXAMPLE 1

The following examples describe the biological action of the compounds according to the invention:

PLK Enzyme Assay

Recombinant human Plk-1 (6×His) was purified from baculovirus-infected insect cells (Hi5).

10 ng of (produced in a recombinant manner and purified) PLK enzyme is incubated for 90 minutes at room temperature with biotinylated casein and 33P-γ-ATP as a substrate in a volume of 15 μl in 384-well Greiner small-volume microtiter plates (final concentrations in the buffer: 660 ng/ml of PLK; 0.7 μmol of casein, 0.5 μmol of ATP incl. 400 nCi/ml of 33P-γ-ATP; 10 mmol of MgCl2, 1 mmol of MnCl2; 0.01% NP40; 1 mmol of DTT, protease inhibitors; 0.1 mmol of Na2VO3 in 50 mmol of HEPES, pH 7.5). To complete the reaction, 5 μl of stop solution (500 μmol of ATP; 500 mmol of EDTA; 1% Triton X100; 100 mg/ml of streptavidin-coated SPA beads in PBS) is added. After the microtiter plate is sealed by film, the beads are sedimented by centrifuging (10 minutes, 1500 rpm). The incorporation of 33P-γ-ATP in casein is intended as a measurement of enzyme activity by β-counting. The extent of the inhibitor activity is referenced against a solvent control (=uninhibited enzyme activity=0% inhibition) and the mean value of several batches that contained 300 μmol of wortmannin (=completely inhibited enzyme activity=100% inhibition).

Test substances are used in various concentrations (0 μmol, as well as in the range of 0.01-30 ∥mol). The final concentration of the solvent dimethyl sulfoxide is 1.5% in all batches.

Proliferation Assay

Cultivated human MaTu breast tumor cells were flattened out at a density of 5000 cells/measuring point in a 96-well multititer plate in 200 μl of the corresponding growth medium. After 24 hours, the cells of one plate (zero-point plate) were colored with crystal violet (see below), while the medium of the other plates was replaced by fresh culture medium (200 μl), to which the test substances were added in various concentrations (0 μm, as well as in the range of 0.01-30 μm; the final concentration of the solvent dimethyl sulfoxide was 0.5%). The cells were incubated for 4 days in the presence of test substances. The cell proliferation was determined by coloring the cells with crystal violet: the cells were fixed by adding 20 μl/measuring point of an 11% glutaric aldehyde solution for 15 minutes at room temperature. After three washing cycles of the fixed cells with water, the plates were dried at room temperature. The cells were colored by adding 100 μl/measuring point of a 0.1% crystal violet solution (pH was set at 3 by adding acetic acid). After three washing cycles of the colored cells with water, the plates were dried at room temperature. The dye was dissolved by adding 100 μl/measuring point of a 10% acetic acid solution. The extinction was determined by photometry at a wavelength of 595 nm. The change of cell growth, in percent, was calculated by standardization of the measured values to the extinction values of the zero-point plate (=0%) and the extinction of the untreated (0 μm) cells (=100%).

The results of the PLK enzyme assay are presented in Table 1 below:

TABLE 1

| | | PLK |
|---|---|---|
| Example | | IC50 |
| No. | Structure | [nM] |
| 25 | 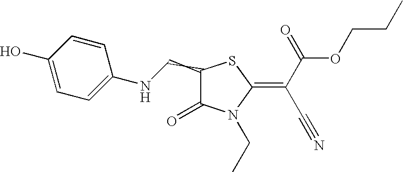 | 36 |
| 7 | 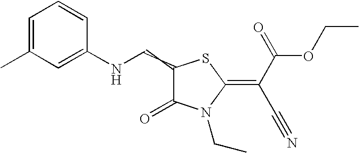 | 46 |
| 36 | 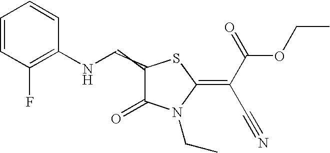 | 160 |
| 19 | 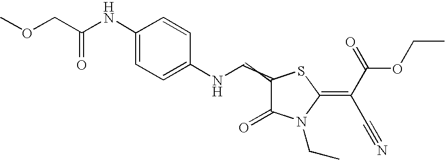 | 500 |
| 56 | 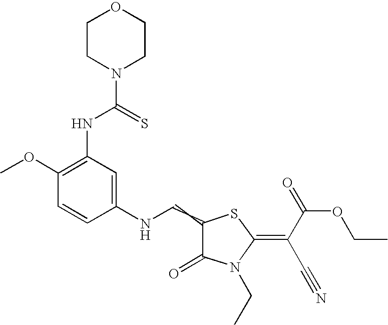 | 810 |
| 234 | 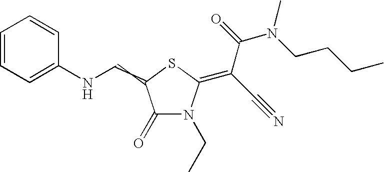 | 950 |
| 223 | 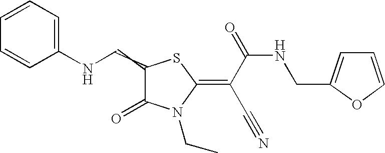 | 3100 |

The results of other PLK enzyme assays and the proliferation assay are presented in Table 2 and 3 below:

TABLE 2
Amides

| | | | Inhibition of Tumor |
|---|---|---|---|
| Example | | Inhibition of Plk-1 | Cell Proliferation |
| No. | Structure | IC50 [nM] | (MaTu) IC50 [μM] |
| 527 | 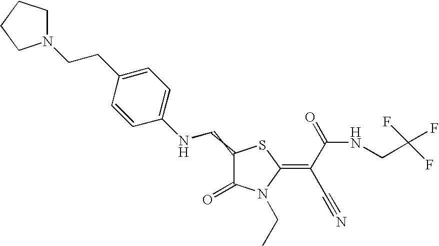 | 100 | 2.8 |
| 310 | 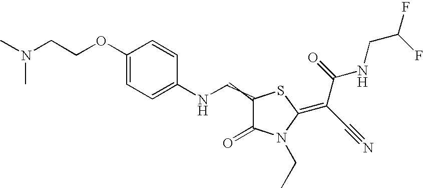 | 74 | 5.6 |
| 307 | 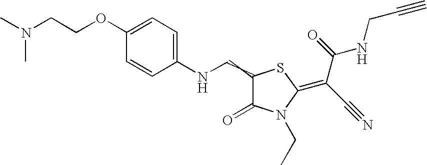 | 71 | 1.7 |
| 330 | 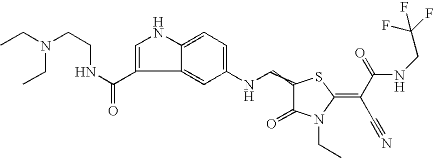 | 41 | 1.2 |
| 169 | 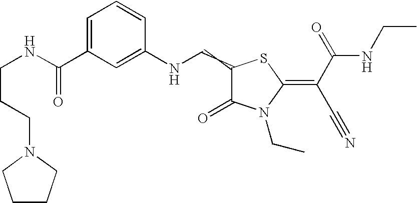 | 345 | 3.55 |
| 192 | 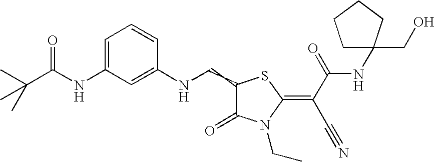 | 190 | 9.7 |
| 210 | 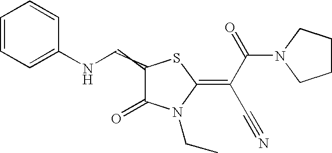 | 270 | 4.5 |

TABLE 3
Esters

| | | | Inhibition of Tumor |
|---|---|---|---|
| Example | | Inhibition of Plk-1 | Cell Proliferation |
| No. | Structure | IC50 [nM] | (MaTu) IC50 [μM] |
| 133 | 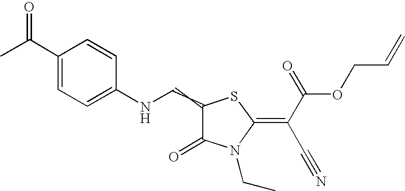 | 34 | 1.4 |
| 132 | 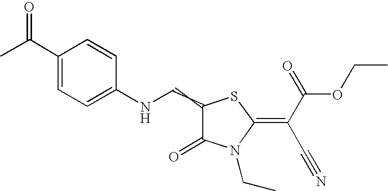 | 81 | 3.1 |
| 47 | 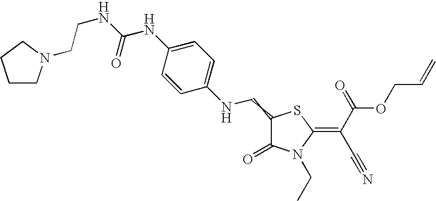 | 23 | 1.1 |
| 74 | 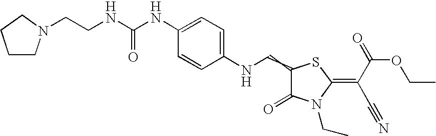 | 37 | 3.3 |

Tables 1 to 3 show that the compounds according to the invention inhibit PLK in the nanomolar range.

### DESCRIPTION OF THE FIGURE

FIG. 1 shows the function of Plk-1

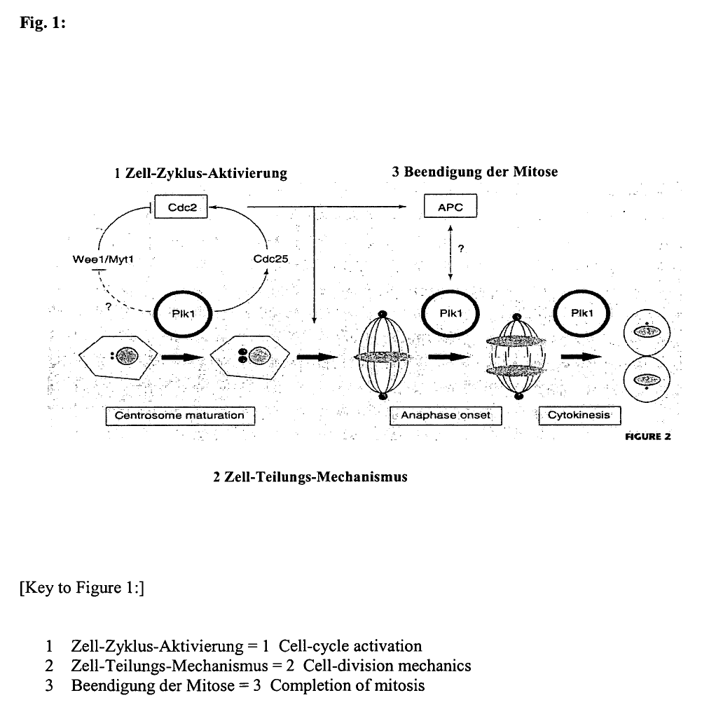

Here:
1. Entry into mitosis: Plk-1 activates CDC25 C. This results in the activation of the CDK/cyclin B complex and converts the cell from G2 to M-status.
2. Triggering of mitosis: Plk 1 plays an important role during the cytokinesis, especially in the formation of the bipolar spindle apparatus and the chromosome separation during the late mitosis phase. Plk-1 is also required during centrosome maturation and binds to so-called ‘kinesin motors.’
3. Completion of mitosis: Plk-1 activates the APC/C complex (anaphase promoting complex/cyclosome; Kotani et al. 1998). APC/C catalyzes as E3-enzyme the polyubiquitinylation of specific substrates, such as, e.g., cyclin B. Such an ubiquitinylation of proteins ultimately results in their degradation into proteasomes. This in turn leads to a reduction of cell-cycle regulators below a critical value and in the exit from the mitosis phase in the so-called G1-status of the cell (M→G1 transition).

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius and, all parts and percentages are by weight, unless otherwise indicated.

The entire disclosures of all applications, patents and publications, cited herein and of corresponding Germany Application No. 10351744.8-44, filed Oct. 31, 2003, and U.S. Provisional Application Ser. No. 60/517,061, filed Nov. 5, 2003 are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

LENGTHY TABLE
The patent application contains a lengthy table section. A copy of the table is available in electronic form from the USPTO web site (http://seqdata.uspto.gov/?pageRequest=docDetail&DocID=US20070037862A1) An electronic copy of the table will also be available from the USPTO upon request and payment of the fee set forth in 37 CFR 1.19(b)(3).

1. Compounds of general formula I 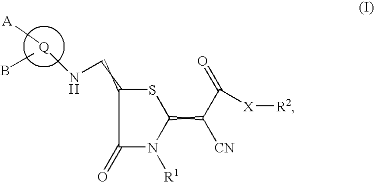
in which
Q stands for aryl or heteroaryl,
A and B, independently of one another, stand for hydrogen, halogen, hydroxy, ammo or nitro;
or
for C₁-C₃-alkyl or C₁-C₆-alkoxy that is optionally substituted in one or more places, in the same way or differently, with halogen, hydroxy, C₃-C₆-heterocycloalkyl or with the group —NR³R⁴ or —CO(NR³)-M, whereby the heterocycloalkyl itself optionally can be interrupted by one or more nitrogen, oxygen and/or sulfur atoms and/or optionally can be interrupted by one or more —(CO)— or —SO₂— groups in the ring, and/or optionally one or more double bonds can be contained in the ring, and/or the ring itself optionally can be substituted in one or more places, in the same way or differently, with C₁-C₆-alkyl, C₃-C₆-cycloalkyl, C₁-C₆-hydroxyalkyl or with the group —NR³R⁴,
or
for —NR³(Co)-L, —NR³(CO)—NR³-L, —COR⁶, —CO(NR³)-M, —NR³(CS)NR³R⁴, —NR³SO₂-M, —SO₂—NR³R⁴ or —SO₂(NR³)-M,
L stands for C₁-C₆-alkyl or heteroaryl that is optionally substituted in one or more places, in the same way or differently, with C₁-C₆-hydroxyalkoxy, C₁-C₆-alkoxyalkoxy, C₃-C₆-heterocycloalkyl or with the group —NR³R⁴, whereby the heterocycloalkyl itself optionally can be interrupted by one or more nitrogen, oxygen and/or sulfur atoms, and/or optionally can be interrupted by one or more —(CO)— or —SO₂— groups in the ring, and/or optionally one or more double bonds can be contained in the ring, and/or the ring itself optionally can be substituted in one or more places, in the same way or differently, with C₁-C₆-alkyl, C₃-C₆-cycloalkyl, C₁-C₆-hydroxyalkyl or with the group —NR³R⁴,
M stands for C₁-C₆-alkyl that is optionally substituted in one or more places, in the same way or differently, with the group —NR³R⁴ or C₃-C₆-heterocycloalkyl,
X stands for —NH— or —NR⁵—,
R¹ stands for C₁-C₄-alkyl, C₃-cycloalkyl, allyl or propargyl that is optionally substituted in one or more places, in the same way or differently, with halogen,
R² stands for hydrogen or for C₁-C₆-alkyl, C₁-C₆-alkoxy, C₁-C₆-alkenyl, C₁-C₆-alkinyl, C₃-C₆-cycloalkyl, C₃-C₆-heterocycloalkyl, aryl or heteroaryl that is optionally substituted in one or more places, in the same way or differently, with halogen, hydroxy, cyano, C₁-C₆-alkyl, C₁-C₆-alkoxy, C₁-C₆-hydroxyalkyl, C₃-C₆-cycloalkyl, C₃-C₆-heterocycloalkyl, C₁-C₆-alkinyl, aryl, aryloxy, heteroaryl or with the group —S—C₁-C₆-alkyl, —COR⁶, —NR³R⁴, —NR³(CO)-L or —NR³COOR⁷, whereby the heterocycloalkyl itself optionally can be interrupted by one or more nitrogen, oxygen and/or sulfur atoms, and/or optionally can be interrupted by one or more —(CO)— or —SO₂— groups in the ring, and/or optionally one or more double bonds can be contained in the ring, and whereby aryl, heteroaryl, C₃-C₆-cycloalkyl- and/or the C₃-C₆-heterocycloalkyl ring in each case itself optionally can be substituted in one or more places, in the same way or differently, with cyano, halogen, hydroxy, C₁-C₆-alkyl, C₁-C₆-hydroxyalkyl or C₁-C₆-alkoxy, C₃-C₆-cycloalkyl, C₃-C₆-heterocycloalkyl, aryl, benzyl or heteroaryl that is optionally substituted in one or more places, in the same way or differently, with halogen,
or
for the group —NR³R⁴, —NR³(CO)-L, or —NR³(CS)NR³R⁴,
or
R² and R⁵ together form a C₃-C₆-heterocycloalkyl ring, which is interrupted at least one time by nitrogen and optionally can be interrupted in one or more places by oxygen or sulfur and/or optionally can be interrupted by one or more —(CO)— or —SO₂— groups in the ring, and/or optionally one or more double bonds can be contained in the ring, and/or the ring itself optionally can be substituted in one or more places, in the same way or differently, with cyano, halogen, hydroxy, C₁-C₆-alkyl, C₃-C₆-cycloalkyl, C₁-C₆-hydroxyalkyl, C₁-C₆-alkoxyalkyl or with the group —NR³R⁴ or —COR⁶ and/or can be substituted with aryl or heteroaryl that is optionally substituted in one or more places, in the same way or differently, with halogen, C₁-C₆-alkoxy or with the group —COR⁶,
R³ and R⁴, independently of one another, stand for hydrogen or for C₁-C₆-alkyl, C₁-C₆-alkoxy, —CO—C₁-C₆-alkyl or aryl that is optionally substituted in one or more places, in the same way or differently, with halogen, hydroxy, C₃-C₆-heterocycloalkyl, C₁-C₆-hydroxyalkoxy or with the group —NR³R⁴, whereby the heterocycloalkyl itself optionally can be interrupted by one or more nitrogen, oxygen and/or sulfur atoms, and/or optionally can be interrupted by one or more —(CO)— or —SO₂— groups in the ring, and/or optionally one or more double bonds can be contained in the ring, and whereby the C₃-C₆-heterocycloalkyl ring itself in each case optionally can be substituted in one or more places, in the same way or differently, with cyano, halogen, C₁-C₆-alkyl, C₁-C₆-hydroxyalkyl, C₁-C₆-alkoxy, C₃-C₆-cycloalkyl, or with the group
—NR³R⁴ or —O—NR³R⁴,
or
R³ and R⁴ together form a C₃-C₆-heterocycloalkyl ring, which is interrupted at least once by nitrogen and optionally can be interrupted in one or more places by oxygen or sulfur and/or optionally can be interrupted by one or more —(CO)— or —SO₂— groups in the ring, and/or optionally one or more double bonds can be contained in the ring,
and/or the heterocycloalkyl ring itself optionally can be substituted in one or more places, in the same way or differently, with C₁-C₆-alkyl, C₃-C₆-cycloalkyl, C₁-C₆-hydroxyalkyl, C₁-C₆-alkoxyalkyl, cyano, hydroxy or with the group —NR³R⁴,
R⁵ stands for C₁-C₆-alkyl, C₁-C₆-alkenyl, or C₁-C₆-alkinyl that is optionally substituted in one or more places, in the same way or differently, with halogen, hydroxy, cyano, C₁-C₆-alkoxy, C₃-C₆-cycloalkyl, C₃-C₆-heterocycloalkyl, or with the group —NR³R⁴, whereby the heterocycloalkyl itself optionally can be interrupted by one or more nitrogen, oxygen and/or sulfur atoms, and/or optionally can be interrupted by one or more —(CO)— or —SO₂— groups in the ring, and/or optionally one or more double bonds can be contained in the ring, and whereby the C₃-C₆-heterocycloalkyl ring itself in each case optionally can be substituted in one or more places, in the same way or differently, with cyano, halogen, C₁-C₆-alkyl, C₁-C₆-hydroxyalkyl, C₁-C₆-alkoxy, C₃-C₆-cycloalkyl, or with the group —NR³R⁴ or —CO—NR³R^{e},
R⁶ stands for hydroxy, C₁-C₆-alkyl, C₁-C₆-alkoxy or the group —NR³R⁴,
R⁷ stands for —(CH₂)ₙ-aryl or —(CH₂)ₙ-heteroaryl and
n stands for 1-6,
as well as their stereoisomers, diastereomers, enantiomers and salts, with the stipulation that the following compounds do not fall under general formula (I):
{2-Cyano-2-[3-ethyl-4-oxo-5-[1-phenylamino-meth-(E/Z)-ylidene]-thiazolidin-(2-(E or Z))-ylidene]-acetylamino}-acetic acid methyl ester,
2-Cyano-2-[3-ethyl-4-oxo-5-[1-phenylamino-meth-(E/Z)-ylidene]-thiazolidin-(2-(E or Z))-ylidene]-N-pyridin-3-ylmethyl-acetamide,
2-Cyano-2-[3-ethyl-4-oxo-5-[1-phenylamino-meth-(E/Z)-ylidene]-thiazolidin-(2-(E or Z))-ylidene]-N-(3-imidazol-1-yl-propyl)-acetamide,
2-Cyano-2-[3-ethyl-4-oxo-5-[1-phenylamino-meth-(E/Z)-ylidene]-thiazolidin-(2-(E or Z))-ylidene]-N-(4-fluoro-benzyl)-acetamide,
2-Cyano-2-[3-ethyl-4-oxo-5-[1-phenylamino-meth-(E/Z)-ylidene]-thiazolidin-(2-(E or Z))-ylidene]-N-(3-morpholin-4-yl-propyl)-acetamide,
2-Cyano-2-[3-ethyl-4-oxo-5-[1-phenylamino-meth-(E/4)-ylidene]-thiazolidin-(2-(E or Z))-ylidene]-N-(2-morpholin-4-yl-ethyl)-acetamide,
2-Cyano-2-[3-ethyl-4-oxo-5-[1-phenylamino-meth-(E/Z)-ylidene]-thiazolidin-(2-(E or Z))-ylidene]-N-[3-(2-oxo-pyrrolidin-1-yl)-propyl]-acetamide,
2-Cyano-N-cyclohexyl-2-[3-ethyl-4-oxo-5-[1-phenylamino-meth-(E/Z)-ylidene]-thiazolidin-(2-(E or Z))-ylidene]-acetamide,
4-{2-Cyano-2-[3-ethyl-4-oxo-5-[1-phenylamino-meth-(E/Z)-ylidene]-thiazolidin-(2-(E or Z))-ylidene]-acetylamino}-piperidine-1-carboxylic acid ethyl ester,
2-Cyano-2-[3-ethyl-4-oxo-5-[1-phenylamino-meth-(E/Z)-ylidene]-thiazolidin-(2-(E or Z))-ylidene]-N-(3-hydroxy-propyl)-acetamide,
2-Cyano-2-[3-ethyl-4-oxo-5-[1-phenylamino-meth-(E/Z)-ylidene]-thiazolidin-(2-(E or Z))-ylidene]-N-(4-methoxy-benzyl)-acetamide,
2-Cyano-2-[3-ethyl-4-oxo-5-[1-phenylamino-meth-(E/Z)-ylidene]-thiazolidin-(2-(E or Z))-ylidene]-N-[2-(4-hydroxy-phenyl)-ethyl]-acetamide,
N-Allyl-2-cyano-2-[3-ethyl-4-oxo-5-[1-phenylamino-meth-(E/Z)-ylidene]-thiazolidin-(2-(E or Z))-ylidene]-acetamide,
2-Cyano-2-[3-ethyl-4-oxo-5-[1-phenylamino-meth-(E/Z)-ylidene]-thiazolidin-(2-(E or Z))-ylidene]-N-(2-hydroxy-ethyl)-acetamide,
2-Cyano-2-[3-ethyl-4-oxo-5-[1-phenylamino-meth-(E/Z)-ylidene]-thiazolidin-(2-(E or Z))-ylidene]-N-(4-hydroxy-butyl)-acetamide,
2-Cyano-2-[3-ethyl-4-oxo-5-[1-phenylamino-meth-(E/Z)-ylidene]-thiazolidin-(2-(E or Z))-ylidene]-N-(6-hydroxy-hexyl)-acetamide,
2-Cyano-2-[3-ethyl-4-oxo-5-[1-phenylamino-meth-(E/Z)-ylidene]-thiazolidin-(2-(E or Z))-ylidene]-acetamide,
2-Cyano-N-ethyl-2-[3-ethyl-4-oxo-5-[1-phenylamino-meth-(E/Z)-ylidene]-thiazolidin-(2-(E or Z))-ylidene]-acetamide,
2-Cyano-2-[3-ethyl-5-[1-(4-methoxy-phenylamino)-meth-(E/Z)-ylidene]-4-oxo-thiazolidin-(2-(E or Z))-ylidene]-N,N-dimethyl-acetamide,
2-Cyano-2-[3-ethyl-4-oxo-5-[1-phenylamino-meth-(E/Z)-ylidene]-thiazolidin-(2-(E or Z))-ylidene]-N,N-dimethyl-acetamide,
6-{[2-[1-Cyano-1-dimethylcarbamoyl-meth-(E or Z)-ylidene]-3-ethyl-4-oxo-thiazolidin-(5-(E/Z))-ylidenemethyl]-amino}-naphthalene-2-carboxylic acid,
4-{[2-[1-Cyano-1-dimethylcarbamoyl-meth-(E or Z)-ylidene]-3-ethyl-4-oxo-thiazolidin-(5-(E/Z))-ylidenemethyl]-amino}-benzoic acid,
2-Cyano-2-[3-ethyl-5-[1-(4-hydroxy-phenylamino)-meth-(E/Z)-ylidene]-4-oxo-thiazolidin-(2-(E or Z))-ylidene]-N,N-dimethyl-acetamide,
4-{[2-[1-Cyano-1-dimethylcarbamoyl-meth-(E or Z)-ylidene]-3-ethyl-4-oxo-thiazolidin-(5-(E/Z))-ylidenemethyl]-amino}-benzamide,
2-Cyano-2-[3-ethyl-5-[1-(4-hydroxymethyl-phenylamino)-meth-(E/Z)-ylidene]-4-oxo-thiazolidin-(2-(E or Z))-ylidene]-N,N-dimethyl-acetamide.

2. Compounds of general formula I, according to claim 1, in which
Q stands for phenyl, naphthyl, quinolinyl, benzimidazolyl, indolyl, indazolyl, thiazolyl, imidazolyl or pyridyl,
A and B, independently of one another, stand for hydrogen, halogen, hydroxy, amino or nitro
or
for C₁-C₃-alkyl or C₁-C₆-alkoxy that is optionally substituted in one or more places, in the same way or differently, with halogen, hydroxy, C₃-C₆-heterocycloalkyl or with the group —NR³R⁴ or —CO(NR³)-M, whereby the heterocycloalkyl itself optionally can be interrupted by one or more nitrogen, oxygen and/or sulfur atoms, and/or optionally can be interrupted by one or more —(CO)— or —SO₂— groups in the ring, and/or optionally one or more double bonds can be contained in the ring, and/or the ring itself optionally can be substituted in one or more places, in the same way or differently, with C₁-C₆-alkyl, C₃-C₆-cycloalkyl, C₁-C₆-hydroxyalkyl or with the group —NR³R⁴,
or,
for —NR³(CO)-L, —NR³(CO)—NR³-L, —COR⁶, —CO(NR³)-M, —NR³(CS)NR³R⁴, —NR³SO₂-M, —SO₂—NR³R⁴ or —SO₂(NR³)-M,
L stands for C₁-C₆-alkyl or heteroaryl that is optionally substituted in one or more places, in the same way or differently, with C₁-C₆-hydroxyalkoxy, C₁-C₆-alkoxyalkoxy, C₃-C₆-heterocycloalkyl or with the group —NR³R⁴, whereby the heterocycloalkyl itself optionally can be interrupted by one or more nitrogen, oxygen and/or sulfur atoms, and/or optionally can be interrupted by one or more —(CO)— or —SO₂— groups in the ring, and/or optionally one or more double bonds can be contained in the ring, and/or the ring itself optionally can be substituted in one or more places, in the same way or differently, with C₁-C₆-alkyl, C₃-C₆-cycloalkyl, C₁-C₆-hydroxyalkyl or with the group —NR³R⁴,
M stands for C₁-C₆-alkyl that is optionally substituted in one or more places, in the same way or differently, with the group —NR³R⁴ or C₃-C₆-heterocycloalkyl,
X stands for —NH— or —NR⁵—,
R¹ stands for C₁-C₄-alkyl, C₃-cycloalkyl, allyl or propargyl that is optionally substituted in one or more places, in the same way or differently, with halogen,
R² stands for hydrogen or for C₁-C₆-alkyl, C₁-C₆-alkoxy, C₁-C₆-alkenyl, C₁-C₆-alkinyl, C₃-C₆-Cycloalkyl, C₃-C₆-heterocycloalkyl, aryl or heteroaryl that is optionally substituted in one or more places, in the same way or differently, with halogen, hydroxy, cyano, C₁-C₆-alkyl, C₁-C₆-alkoxy, C₁-C₆-hydroxyalkyl, C₃-C₆-cycloalkyl, C₃-C₆-heterocycloalkyl, C₁-C₆-alkinyl, aryl, aryloxy, heteroaryl or with the group —S—C₁-C₆-alkyl, —COR⁶, —NR³R⁴, —NR³(CO)-L or —NR³COOR⁷, whereby the heterocycloalkyl itself optionally can be interrupted by one or more nitrogen, oxygen and/or sulfur atoms, and/or optionally can be interrupted by one or more —(CO)— or —SO₂— groups in the ring, and/or optionally one or more double bonds can be contained in the ring,
and whereby aryl, heteroaryl, C₃-C₆-cycloalkyl- and/or the C₃-C₆-heterocycloalkyl ring in each case itself optionally can be substituted in one or more places, in the same way or differently, with cyano, halogen, hydroxy, C₁-C₆-alkyl, C₁-C₆-hydroxyalkyl, or C₁-C₆-alkoxy, C₃-C₆-cycloalkyl, C₃-C₆-heterocycloalkyl, aryl, benzyl or heteroaryl that is optionally substituted in one or more places, in the same way or differently, with halogen,
or
for the group —NR³R⁴, —NR³(CO)-L, or —NR³(CS)NR³R⁴,
or
R² and R⁵ together form a C₃-C₆-heterocycloalkyl ring, which is interrupted at least once by nitrogen and optionally can be interrupted in one or more places by oxygen or sulfur and/or optionally can be interrupted by one or more —(CO)— or —SO₂— groups in the ring, and/or optionally one or more double bonds can be contained in the ring, and/or the ring itself optionally can be substituted in one or more places, in the same way or differently, with cyano, halogen, hydroxy, C₁-C₆-alkyl, C₃-C₆-cycloalkyl, C₁-C₆-hydroxyalkyl, C₁-C₆-alkoxyalkyl or with the group —NR³R⁴ or —COR⁶, and/or with aryl or heteroaryl that is optionally substituted in one or more places, in the same way or differently, with halogen, C₁-C₆-alkoxy or with the group —COR⁶,
R³ and R⁴, independently of one another, stand for hydrogen or for C₁-C₆-alkyl, C₁-C₆-alkoxy, —CO—C₁-C₆-alkyl or aryl that is optionally substituted in one or more places, in the same way or differently, with halogen, hydroxy, C₃-C₆-heterocycloalkyl, C₁-C₆-hydroxyalkoxy or with the group —NR³R⁴, whereby the heterocycloalkyl itself optionally can be interrupted by one or more nitrogen, oxygen and/or sulfur atoms, and/or optionally can be interrupted by one or more —(CO)— or —SO₂— groups in the ring, and/or optionally one or more double bonds can be contained in the ring, and whereby the C₃-C₆-heterocycloalkyl ring itself in each case optionally can be substituted in one or more places, in the same way or differently, with cyano, halogen, C₁-C₆-alkyl, C₁-C₆-hydroxyalkyl, C₁-C₆-alkoxy, C₃-C₆-cycloalkyl, or with the group
—NR³R⁴ or —CO—NR³R^{e},
or
R³ and R⁴ together form a C₃-C₆-heterocycloalkyl ring, which is interrupted by nitrogen at least once and optionally can be interrupted in one or more places by oxygen or sulfur, and/or optionally can be interrupted by one or more —(CO)— or —SO₂— groups in the ring, and/or optionally one or more double bonds can be contained in the ring, and/or the heterocycloalkyl ring itself optionally can be substituted in one or more places, in the same way or differently, with C₁-C₆-alkyl, C₃-C₆-cycloalkyl, C₁-C₆-hydroxyalkyl, C₁-C₆-alkoxyalkyl, cyano, hydroxy or with the group —NR³R⁴,
R⁵ stands for C₁-C₆-alkyl, C₁-C₆-alkenyl, or C₁-C₆-alkinyl that is optionally substituted in one or more places, in the same way or differently, with halogen, hydroxy, cyano, C₁-C₆-alkoxy, C₃-C₆-cycloalkyl, C₃-C₆-heterocycloalkyl, or with the group —NR³R⁴, whereby the heterocycloalkyl itself optionally can be interrupted by one or more nitrogen, oxygen and/or sulfur atoms and/or optionally can be interrupted by one or more —(CO)— or —SO₂— groups in the ring, and/or optionally one or more double bonds can be contained in the ring, and whereby the C₃-C₆-heterocycloalkyl ring itself in each case optionally can be substituted in one or more places, in the same way or differently, with cyano, halogen, C₁-C₆-alkyl, C₁-C₆-hydroxyalkyl, C₁-C₆-alkoxy, C₃-C₆-cycloalkyl, or with the group —NR³R⁴ or —CO—NR³R^{e},
R⁶ stands for hydroxy, C₁-C₆-alkyl, C₁-C₆-alkoxy or the group —NR³R⁴,
R⁷ stands for —(CH₂)ₙ-aryl or —(CH₂)ₙ-heteroaryl and
n stands for 1-6,
as well as their solvates, hydrates, stereoisomers, diastereomers, enantiomers and salts.

3. Compounds of general formula I, according to claim 1 or I, in which
Q stands for phenyl, naphthyl or indolyl,
A and B, independently of one another, stand for hydrogen, halogen, hydroxy, amino or nitro
or
for C₁-C₃-alkyl or C₁-C₆-alkoxy that is optionally substituted in one or more places, in the same way or differently, with halogen, hydroxy, C₃-C₆-heterocycloalkyl or with the group —NR³R⁴ or —CO(NR³)-M, whereby the heterocycloalkyl itself optionally can be interrupted by one or more nitrogen, oxygen and/or sulfur atoms, and/or optionally can be interrupted by one or more —(CO)— or —SO₂— groups in the ring, and/or optionally one or more double bonds can be contained in the ring, and/or the ring itself optionally can be substituted in one or more places, in the same way or differently, with C₁-C₆-alkyl, C₃-C₆-cycloalkyl, C₁-C₆-hydroxyalkyl or with the group —NR³R⁴,
or
for —NR³(CO)-L, —NR³(CO)—NR³-L, —COR⁶, —CO(R³)-M, —NR³(CS)NR³R⁴, —NR³SO₂-M, —SO₂—NR³R⁴ or —SO₂(NR³)-M,
L stands for C₁-C₆-alkyl or heteroaryl that is optionally substituted in one or more places, in the same way or differently, with C₁-C₆-hydroxyalkoxy, C₁-C₆-alkoxyalkoxy, C₃-C₆-heterocycloalkyl or with the group —NR³R⁴, whereby the heterocycloalkyl itself optionally can be interrupted by one or more nitrogen, oxygen and/or sulfur atoms, and/or optionally can be interrupted by one or more —(CO)— or —SO₂— groups in the ring, and/or optionally one or more double bonds can be contained in the ring, and/or the ring itself optionally can be substituted in one or more places, in the same way or differently, with C₁-C₆-alkyl, C₃-C₆-cycloalkyl, C₁-C₆-hydroxyalkyl or with the group —NR³R⁴,
M stands for C₁-C₆-alkyl that is optionally substituted in one or more places, in the same way or differently, with the group —NR³R⁴ or C₃-C₆-heterocycloalkyl,
X stands for —NH— or —NR^{c}—,
R¹ stands for C₁-C₄-alkyl, C₃-cycloalkyl, allyl or propargyl that is optionally substituted in one or more places, in the same way or differently, with halogen,
R² stands for hydrogen or for C₁-C₆-alkyl, C₁-C₆-alkoxy, C₁-C₆-alkenyl, C₁-C₆-alkinyl, C₃-C₆-Cycloalkyl, C₃-C₆-heterocycloalkyl, aryl or heteroaryl that is optionally substituted in one or more places, in the same way or differently, with halogen, hydroxy, cyano, C₁-C₆-alkyl, C₁-C₆-alkoxy, C₁-C₆-hydroxyalkyl, C₃-C₆-cycloalkyl, C₃-C₆-heterocycloalkyl, C₁-C₆-alkinyl, aryl, aryloxy, heteroaryl or with the group —S—C₁-C₆-alkyl, —COR⁶, —NR³R⁴, —NR³(CO)-L or —NR³COOR⁷,
whereby the heterocycloalkyl itself optionally can be interrupted by one or more nitrogen, oxygen and/or sulfur atoms and/or optionally can be interrupted by one or more —(CO)— or —SO₂— groups in the ring, and/or optionally one or more double bonds can be contained in the ring,
and whereby aryl, heteroaryl, C₃-C₆-cycloalkyl- and/or the C₃-C₆-heterocycloalkyl ring in each case itself optionally can be substituted in one or more places, in the same way or differently, with cyano, halogen, hydroxy, C₁-C₆-alkyl, C₁-C₆-hydroxyalkyl, or C₁-C₆-alkoxy, C₃-C₆-cycloalkyl, C₃-C₆-heterocycloalkyl, aryl, benzyl or heteroaryl that is optionally substituted in one or more places, in the same way or differently, with halogen,
or
for the group —NR³R⁴, —NR³(CO)-L, or —NR³(CS)NR³R⁴,
or
R² and R⁵ together form a C₃-C₆-heterocycloalkyl ring, Which is interrupted at least once by nitrogen and optionally can be interrupted in one or more places by oxygen or sulfur and/or optionally can be interrupted by one or more —(CO)— or —SO₂— groups in the ring, and/or optionally one or more double bonds can be contained in the ring, and/or the ring itself optionally can be substituted in one or more places, in the same way or differently, with cyano, halogen, hydroxy, C₁-C₆-alkyl, C₃-C₆-cycloalkyl, C₁-C₆-hydroxyalkyl, C₁-C₆-alkoxyalkyl or with the group —NR³R⁴ or —COR⁶, and/or can be substituted with aryl or heteroaryl that is optionally substituted in one or more places, in the same way or differently, with halogen, C₁-C₆-alkoxy or with the group
—COR⁶,
R³ and R⁴, independently of one another, stand for hydrogen or for C₁-C₆-alkyl, C₁-C₆-alkoxy, —CO—C₁-C₆-alkyl or aryl that is optionally substituted in one or more places, in the same way or differently, with halogen, hydroxy, C₃-C₆-heterocycloalkyl, C₁-C₆-hydroxyalkoxy or with the group —NR³R⁴, whereby the heterocycloalkyl itself optionally can be interrupted by one or more nitrogen, oxygen and/or sulfur atoms, and/or optionally can be interrupted by one or more —(CO)— or —SO₂— groups in the ring, and/or optionally one or more double bonds can be contained in the ring, and whereby the C₃-C₆-heterocycloalkyl ring itself in each case optionally can be substituted in one or more places, in the same way or differently, with cyano, halogen, C₁-C₆-alkyl, C₁-C₆-hydroxyalkyl, C₁-C₆-alkoxy, C₃-C₆-cycloalkyl, or with the group
—NR³R⁴ or —CO—NR³R⁴,
or
R³ and R⁴ together form a C₃-C₆-heterocycloalkyl ring, which is interrupted at least once by nitrogen, and optionally can be interrupted in one or more places by oxygen or sulfur, and/or optionally can be interrupted by one or more —(CO)— or —SO₂— groups in the ring, and/or optionally one or more double bonds can be contained in the ring,
and/or the heterocycloalkyl ring itself optionally can be substituted in one or more places, in the same way or differently, with C₁-C₆-alkyl, C₃-C₆-cycloalkyl, C₁-C₆-hydroxyalkyl, C₁-C₆-alkoxyalkyl, cyano, hydroxy or with the group —NR³R⁴,
R⁵ stands for C₁-C₆-alkyl, C₁-C₆-alkenyl, or C₁-C₆-alkinyl that is optionally substituted in one or more places, in the same way or differently, with halogen, hydroxy, cyano, C₁-C₆-alkoxy, C₃-C₆-cycloalkyl, C₃-C₆-heterocycloalkyl, or with the group —NR³R⁴, whereby the heterocycloalkyl itself-optionally can be interrupted by one or more nitrogen, oxygen and/or sulfur atoms, and/or optionally can be interrupted by one or more —(CO)— or —SO₂— groups in the ring, and/or optionally one or more double bonds can be contained in the ring, and whereby the C₃-C₆-heterocycloalkyl ring itself in each case optionally can be substituted in one or more places, in the same way or differently, with cyano, halogen, C₁-C₆-alkyl, C₁-C₆-hydroxyalkyl, C₁-C₆-alkoxy, C₃-C₆-cycloalkyl, or with the group —NR³R⁴ or —CO—NR³R⁴,
R⁶ stands for hydroxy, C₁-C₆-alkyl, C₁-C₆-alkoxy or the group —NR³R⁴,
R⁷ stands for —(CH₂)ₙ-aryl or —(CH₂)ₙ-heteroaryl and
n stands for 1-6,
as well as their solvates, hydrates, stereoisomers, diastereomers, enantiomers and salts.

4. Compounds of general formula I, according to claim 1, in which
Q stands for phenyl, naphthyl or indolyl,
A and B, independently of one another, stand for hydrogen, halogen, hydroxy, amino or nitro
or
for C₁-C₃-alkyl or C₁-C₆-alkoxy that is optionally substituted in one or more places, in the same way or differently, with pyrrolidinyl, piperidinyl, piperazinyl or with the group —N(C₁-C₆-alkyl)₂, whereby pyrrolidinyl, piperidinyl or piperazinyl itself optionally can be substituted in one or more places, in the same way or differently, with C₁-C₆-alkyl or C₁-C₆-hydroxyalkyl,
or
for —CO(NH)-M, —CO(NCH₃)-M, —NH(CO)-L, —NH(CO)—NH-L, —SO₂(NH)-M or —SO₂(NCH₃)-M,
L stands for C₁-C₆-alkyl or pyridyl that is optionally substituted in one or more places, in the same way or differently, with C₁-C₆-hydroxyalkoxy, C₁-C₆-alkoxyalkoxy, pyrrolidinyl, piperazinyl or with the group —N(C₁-C₆-alkyl)₂, whereby the pyrrolidinyl or piperazinyl itself optionally can be substituted in one or more places, in the same way or differently, with C₁-C₆-alkyl,
M stands for C₁-C₆-alkyl that is optionally substituted in one or more places, in the same way or differently, with the group —N(C₁-C₆-alkyl)₂ or pyrrolidinyl,
X stands for —NH— or —NR⁵—,
R¹ stands for C₁-C₄-alkyl that is optionally substituted in one or more places, in the same way or differently, with halogen,
R² stands for hydrogen or for C₁-C₆-alkyl, C₁-C₆-alkenyl, C₁-C₆-alkinyl, C₃-C₆-cycloalkyl, pyrrolidinyl, piperidinyl, phenyl, tetralinyl or indolyl that is optionally substituted in one or more places, in the same way or differently, with halogen, hydroxy, cyano, C₁-C₆-alkyl, C₁-C₆-alkoxy, C₁-C₆-hydroxyalkyl, C₃-C₆-cycloalkyl, tetrahydrofuranyl, pyrrolidinyl, piperazinyl, morpholinyl, phenyl, phenoxy, biphenyl, naphthyl, thienyl, furanyl, tetrazolyl, pyridyl or with the group —S—C₁-C₆-alkyl, —CONH₂, —COO—C₁-C₆-alkyl, —N(C₁-C₆-alkyl)₂, —N(C₁-C₆-alkyl)phenyl, or —NH(CO)-L,
whereby phenyl, furanyl, C₃-C₆-cycloalkyl, piperidinyl or piperazinyl in each case itself optionally can be substituted in one or more places, in the same way or differently, with C₁-C₆-alkyl, C₁-C₆-alkoxy, cyano, halogen, hydroxy, phenyl, benzyl, or morpholinyl, and the C₁-C₆-alkyl or C₁-C₆-alkoxy itself optionally can be substituted in one or more places, in the same way or differently, with halogen, or
for the group —N(C₁-C₆-alkyl)₂, —NH(CO)-L, or —NCH₃(CS)NHCH₃,
or
R² and R⁵ together form aziridinyl, azetidinyl, morpholinyl, pyrrolidinyl, piperidinyl or, piperazinyl, whereby aziridinyl, azetidinyl, morpholinyl, pyrrolidinyl, piperidinyl or piperazinyl itself optionally can be substituted in one or more places, in the same way or differently, with hydroxy, C₁-C₆-alkyl, C₁-C₆-hydroxyalkyl, C₁-C₆-alkoxyalkyl or with the group —CONH₂, O—C₁-C₆-alkyl or —COO—C₁-C₆-alkyl, and/or can be substituted with phenyl, benzyl or pyridyl that is optionally substituted in one or more places, in the same way or differently, with halogen or C₁-C₆-alkoxy, and
R⁵ stands for C₁-C₆-alkyl or C₁-C₆-alkenyl that is optionally substituted in one or more places, in the same way or differently, with C₁-C₆-alkoxy,
as well as their solvates, hydrates, stereoisomers, diastereomers, enantiomers and salts.

5. Compounds of general formula (I), according to claim 1, in which
Q stands for phenyl, naphthyl or indolyl,
A and B, independently of one another, stand for hydrogen, halogen, hydroxy, amino or nitro,
or
for C₁-C₃-alkyl or C₁-C₃-alkoxy that is optionally substituted in one or more places, in the same way or differently, with pyrrolidinyl, piperidinyl, piperazinyl or with the group —N(CH₃)₂ or —CO(NH)—(CH₂)₂—N(CH₃)₂, whereby pyrrolidinyl, piperidinyl or piperazinyl itself optionally can be substituted in one or more places, in the same way or differently, with C₁-C₃-alkyl or C₁-C₃-hydroxyalkyl, or
for the group —CO—NH—(CH₂)₂—N(CH₃)₂, —CO—NH—(CH₂)₂—N(C₂H₅)₂, —CO—N(CH₃)—(CH₂)₂—N(CH₃)₂, 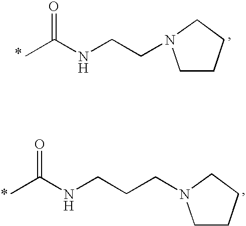
—NH(CO)—C(CH₃)₃, —NH(CO)—(CH₂)—O(CH₂)₂—OCH₃, —NH(CO)—(CH₂)₂—N(C₂H₅)₂, 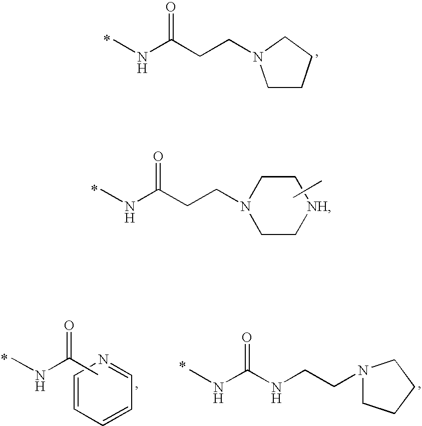
or —SO₂—NH—(CH₂)₂—N(CH₃)₂ or —SO₂—N(CH₃)—(CH₂)₂—N(CH₃)₂,
X stands for —NH— or —NR⁵—,
R¹ stands for C₁-C₃-alkyl that is optionally substituted in one or more places, in the same way or differently, with halogen,
R² stands for hydrogen or for C₁-C₆-alkyl, C₁-C₄-alkenyl, C₁-C₄-alkinyl, C₃-C₆-cycloalkyl, piperidinyl, phenyl, pyrrolidinyl, indolyl or tetralinyl that is optionally substituted in one or more places, in the same way or differently, with halogen, hydroxy, cyano, C₁-C₆-alkyl, C₁-C₆-hydroxyalkyl, methoxy, C₃-C₆-cycloalkyl, tetrahydrofuranyl, pyrrolidinyl, piperazinyl, morpholinyl, phenyl, phenoxy, biphenyl, naphthyl, thienyl, furanyl, tetrazolyl or pyridyl or with the group —S—CH₃, —COOCH₃, —COOC₂H₅, —CO—NH₂, —OCF₃, —N(CH₃)-phenyl, —N(C₁-C₄-alkyl)₂, or —NH(CO)—CH₃, whereby phenyl, furanyl, C₃-C₆-cycloalkyl, piperidinyl or piperazinyl optionally in each case itself can be substituted in one or more places, in the same way or differently, with cyano, halogen, hydroxy, C₁-C₃-alkyl, C₁-C₃-hydroxyalkyl, methoxy, morpholinyl, phenyl or benzyl,
or
for the group —N(CH₃)₂, —N(CH₃)(CS)NHCH₃, —NH(CO)—CH₃, —NH(CO)-pyridyl, or —NH(CO)-pyridinyl,
or
R² and R⁵ together form one of the following rings: 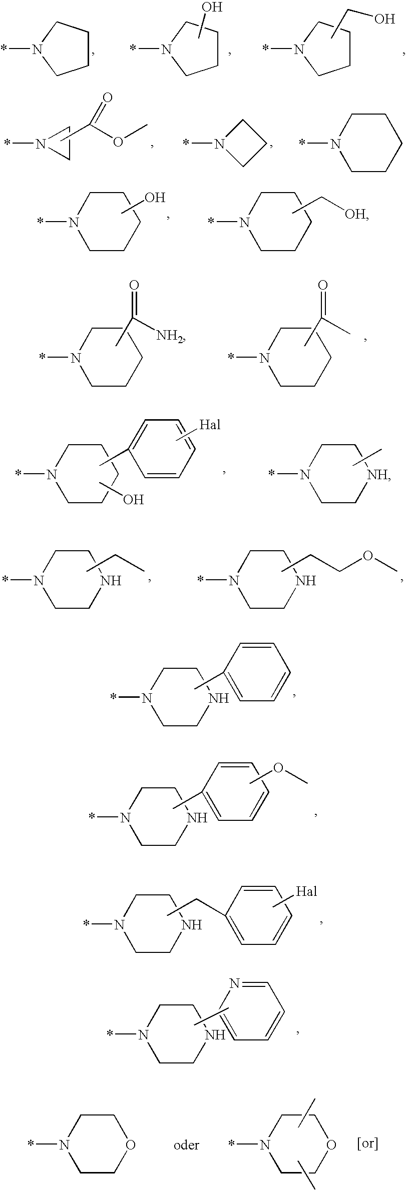
and
R⁵ stands for C₁-C₃-alkyl or C₁-C₃-alkenyl that is optionally substituted in one or more places, in the same way or differently, with C₁-C₆-alkoxy,
as well as their solvates, hydrates, stereoisomers, diastereomers, enantiomers and salts.

6. Compounds of general formula IA
in which 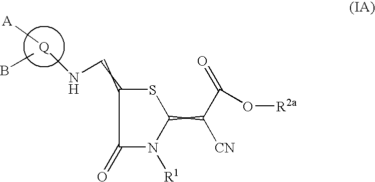
Q stands for aryl or heteroaryl,
A and B, independently of one another, stand for hydrogen, halogen, hydroxy, amino or nitro
or
for C₁-C₃-alkyl or C₁-C₆-alkoxy that is optionally substituted in one or more places, in the same way or differently, with halogen, hydroxy, C₃-C₆-heterocycloalkyl or with the group —NR³R⁴ or —CO(NR³)-M, whereby the heterocycloalkyl itself optionally can be interrupted by one or more nitrogen, oxygen and/or sulfur atoms, and/or optionally can be interrupted by one or more —(CO)— or —SO₂— groups in the ring, and/or optionally one or more double bonds can be contained in the ring, and/or the ring itself optionally can be substituted in one or more places, in the same way or differently, with C₁-C₆-alkyl, C₃-C₆-cycloalkyl, C₁-C₆-hydroxyalkyl or with the group —NR³R⁴,
or
for —NR³(CO)-L, —NR³(CO)—NR³-L, —COR⁶, —CO(NR³)-M, —NR³(CS)NR³R^{e}, —NR³SO₂-M, —SO₂—NR³R⁴ or —SO₂(NR³)-M,
L stands for C₁-C₆-alkyl or heteroaryl that is optionally substituted in one or more places, in the same way or differently, with C₁-C₆-hydroxyalkoxy, C₁-C₆-alkoxyalkoxy, C₃-C₆-heterocycloalkyl or with the group —NR³R⁴, whereby the heterocycloalkyl itself optionally can be interrupted by one or more nitrogen, oxygen and/or sulfur atoms, and/or optionally can be interrupted by one or more —(CO)— or —SO₂— groups in the ring, and/or optionally one or more double bonds can be contained in the ring, and/or the ring itself optionally can be substituted in one or more places, in the same way or differently, with C₁-C₆-alkyl, C₃-C₆-cycloalkyl, C₁-C₆-hydroxyalkyl or with the group —NR³R⁴,
M stands for C₁-C₆-alkyl that is optionally substituted in one or more places, in the same way or differently, with the group —NR³R⁴ or C₃-C₆-heterocycloalkyl,
R¹ stands for C₁-C₄-alkyl, C₃-cycloalkyl, allyl or propargyl that is optionally substituted in one or more places, in the same way or differently, with halogen,
R^{2a} stands for allyl or propargyl,
R³ and R⁴, independently of one another, stand for hydrogen or for C₁-C₆-alkyl, C₁-C₆-alkoxy, —CO—C₁-C₆-alkyl or aryl that is optionally substituted in one or more places, in the same way or differently, with halogen, hydroxy, C₃-C₆-heterocycloalkyl, C₁-C₆-hydroxyalkoxy or with the group —NR³R⁴, whereby the heterocycloalkyl itself optionally can be interrupted by one or more nitrogen, oxygen and/or sulfur atoms, and/or optionally can be interrupted by one or more —(CO)— or —SO₂— groups in the ring, and/or optionally one or more double bonds can be contained in the ring, and whereby the C₃-C₆-heterocycloalkyl ring itself in each case optionally can be substituted in one or more places, in the same way or differently, with cyano, halogen, C₁-C₆-alkyl, C₁-C₆-hydroxyalkyl, C₁-C₆-alkoxy, C₃-C₆-cycloalkyl, or with the group
—NR³R⁴ or —CO—NR³R¹,
or
R³ and R⁴ together form a C₃-C₆-heterocycloalkyl ring, which is interrupted at least once by nitrogen and optionally can be interrupted in one or more places by oxygen or sulfur, and/or optionally can be interrupted by one or more {CO)— or —SO₂— groups in the ring, and/or optionally one or, more double bonds can be contained in the ring,
and/or the heterocycloalkyl ring itself optionally can be substituted in one or more places, in the same way or differently, with C₁-C₆-alkyl, C₃-C₆-cycloalkyl, C₁-C₆-hydroxyalkyl, C₁-C₆-alkoxyalkyl, cyano, hydroxy or with the group —NR³R⁴, and
R⁶ stands for hydroxy, C₁-C₆-alkyl, C₁-C₆-alkoxy or the group —NR³R⁴,
as well as their solvates, hydrates, stereoisomers, diastereomers, enantiomers and salts.

7. Compounds of general formula IA, according to claim 6, in which
Q stands for phenyl, quinolinyl, indolyl or naphthyl,
A and B, independently of one another, stand for hydrogen or halogen,
or
for C₁-C₃-alkyl or C₁-C₆-alkoxy that is optionally substituted in one or more places, in the same way or differently, with halogen, hydroxy or with the group —NC₁-C₆-alkyl)₂ or —CO(NH)-M,
or
for —NH(CO)-L, —NH(CO)—NH-L, —COR⁶, —CO(NH)-M, —CO(NCH₃)-M, —SO₂(NH)-M or —SO₂(NCH₃)-M,
L stands for C₁-C₆-alkyl that is optionally substituted in one or more places, in the same way or differently, with pyrrolidinyl,
M stands for C₁-C₆-alkyl that is optionally substituted in one or more places, in the same way or differently, with the group —N(C₁-C₆-alkyl)₂ or pyrrolidinyl,
R¹ stands for C₁-C₃-alkyl,
R^{2a} stands for allyl or propargyl, and
R⁶ stands for hydroxy, C₁-C₆-alkyl or C₁-C₆-alkoxy,
as well as their solvates, hydrates, stereoisomers, diastereomers, enantiomers and salts.

8. Compounds of the following formulas, as well as their solvates, hydrates, stereoisomers, diastereomers, enantiomers and salts:
(E or Z)-Cyano-(3-ethyl-5-(E/Z)-{[4-(2-morpholin-4-yl-ethanesulfonylamino)-phenylamino]-methylene}-4-oxo-thiazolidin-2-ylidene)-acetic acid ethyl ester,
(E or Z)-Cyano-[3-ethyl-4-oxo-5-(E/Z)-({4-[(pyrrolidine-1-carbonyl)-amino]-phenylamino}-methylene)-thiazolidin-2-ylidene]-acetic acid ethyl ester,
(E or Z)-Cyano-(3-ethyl-5-(E/Z-({4-[3-(4-methyl-piperazin-1-yl)-propionylamino]-phenylamino}-methylene}-4-oxo-thiazolidin-2-ylidene)-acetic acid allyl ester,
(E or Z)-Cyano-(3-ethyl-5-(E/Z-({4-[3-(4-methyl-piperazin-1-yl)-propionylamino]-phenylamino}-methylene}-4-oxo-thiazolidin-2-ylidene)-acetic acid benzyl ester,
(E or Z)-Cyano-(3-ethyl-5-(E/Z)-{[4-(2-pyrrolidin-1-yl-ethylcarbamoyl)-phenylamino]-methylene}-4-oxo-thiazolidin-2-ylidene)-acetic acid allyl ester,
(E or Z)-Cyano-[3-ethyl-4-oxo-5-(E/Z)-(p-tolylamino-methylene)-thiazolidin-2-ylidene]-acetic acid ethyl ester,
(E or Z)-Cyano-[3-ethyl-4-oxo-5-(E/Z)-(m-tolylamino-methylene)-thiazolidin-2-ylidene]-acetic acid ethyl ester,
(E or Z)-Cyano-{3-ethyl-5-(E/Z)-[(3-nitro-phenylamino)-methylene]-4-oxo-thiazolidin-2-ylidene}-acetic acid ethyl ester,
(E or Z)-{5-(E/Z)-[(3-Chloro-phenylamino)-methylene]-3-ethyl-4-oxo-thiazolidin-2-ylidene}-cyano-acetic acid ethyl ester,
5-{[2-((E or Z)-Cyano-ethoxycarbonyl-methylene)-3-ethyl-4-oxo-thiazolidin-5-(E/Z)-ylidenemethyl]-amino}-1H-indole-2-carboxylic acid ethyl ester,
(E or Z)-Cyano-{3-ethyl-5-(E/Z)-[(2-methyl-1H-indol-5-ylamino)-methylene]-4-oxo-thiazolidin-2-ylidene}-acetic acid ethyl ester,
(E or Z)-{5-(E/Z)-[(3-Carbamoyl-1H-indol-5-ylamino)-methylene]-3-ethyl-4-oxo-thiazolidin-2-ylidene}-cyano-acetic acid ethyl ester,
(E or Z)-Cyano-(3-ethyl-5-(E/Z)-{[3-(4-methyl-piperazine-1-carbonyl)-phenylamino]-methylene}-4-oxo-thiazolidin-2-ylidene)-acetic acid ethyl ester,
(E or Z)-Cyano-[3-ethyl-5-(E/Z)-({3-[2-(2-hydroxymethyl-pyrrolidin-1-yl)-ethanesulfonylamino]-phenylamino}-methylene)-4-oxo-thiazolidin-2-ylidene]-acetic acid ethyl ester,
(E or Z)-Cyano-(3-ethyl-4-oxo-5-(E/Z)-{[3-(2-piperidin-1-yl-ethanesulfonylamino)-phenylamino]-methylene}-thiazolidin-2-ylidene)-acetic acid ethyl ester,
(E or Z)-Cyano-(3-ethyl-4-oxo-5-(E/Z)-{[3-(2-pyrrolidin-1-yl-ethanesulfonylamino)-phenylamino]-methylene}-thiazolidin-2-ylidene)-acetic acid ethyl ester,
(E or Z)-Cyano-(3-ethyl-5-(E/Z)-{[4-(3-methoxy-propionylamino)-phenylamino]-methylene}-4-oxo-thiazolidin-2-ylidene)-acetic acid ethyl ester,
(E or Z)-Cyano-[3-ethyl-5-(E/Z)-({4-[2-(2-methoxy-ethoxy)-acetylamino]-phenylamino}-methylene)-4-oxo-thiazolidin-2-ylidene]-acetic acid ethyl ester,
(E or Z)-Cyano-(3-ethyl-5-(E/Z)-{[4-(2-methoxy-acetylamino)-phenylamino]-methylene}-4-oxo-thiazolidin-2-ylidene)-acetic acid ethyl ester,
(E or Z)-Cyano-(3-ethyl-4-oxo-5-(E/Z)-{[4-(2-piperidin-1-yl-ethanesulfonylamino)-phenylamino]-methylene}-thiazolidin-2-ylidene)-acetic acid ethyl ester,
(E or Z)-Cyano-[3-ethyl-5-(E/Z)-({4-[2-(4-methyl-piperazin-1-yl)-ethanesulfonylamino]-phenylamino}-methylene)-4-oxo-thiazolidin-2-ylidene]-acetic acid ethyl ester,
(E or Z)-Cyano-{3-ethyl-5-(E/Z)-[(4-methanesulfonylamino-phenylamino)-methylene]-4-oxo-thiazolidin-2-ylidene}-acetic acid ethyl ester,
(E or Z)-Cyano-[3-ethyl-5-(E/Z)-({4-[2-(2-hydroxymethyl-piperidin-1-yl)-ethanesulfonylamino]-phenylamino}-methylene)-4-oxo-thiazolidin-2-ylidene]-acetic acid ethyl ester,
(E or Z)-Cyano-[3-ethyl-5-(E/Z)-({4-[2-(2-hydroxymethyl-pyrrolidin-1-yl)-ethanesulfonylamino]-phenylamino}-methylene)-4-oxo-thiazolidin-2-ylidene]-acetic acid ethyl ester,
(E or Z)-Cyano-{3-ethyl-5-(E/Z)-[(4-hydroxy-phenylamino)-methylene]-4-oxo-thiazolidin-2-ylidene}-acetic acid propyl ester,
(E or Z)-Cyano-{3-ethyl-5-(E/Z)-[(2-fluoro-4-hydroxy-phenylamino)-methylene]-4-oxo-thiazolidin-2-ylidene}-acetic acid ethyl ester,
(E or Z)-{5-(E/Z)-[(3-Chloro-4-hydroxy-phenylamino)-methylene]-3-ethyl-4-oxo-thiazolidin-2-ylidene}-cyano-acetic acid ethyl ester,
(E or Z)-Cyano-{3-ethyl-5-(E/Z)-[(4-hydroxy-3-nitro-phenylamino)-methylene]-4-oxo-thiazolidin-2-ylidene}-acetic acid ethyl ester,
(E or Z)-Cyano-{5-(E/Z)-[(3,5-dichloro-4-hydroxy-phenylamino)-methylene]-3-ethyl-4-oxo-thiazolidin-2-ylidene}-acetic acid ethyl ester,
(E or Z)-Cyano-{3-ethyl-5-(E/Z)-[(4-hydroxy-3,5-dimethyl-phenylamino)-methylene]-4-oxo-thiazolidin-2-ylidene}-acetic acid ethyl ester,
(E or Z)-Cyano-{5-(E/Z)-[(3-diethylaminomethyl-4-hydroxy-phenylamino)-methylene]-3-ethyl-4-oxo-thiazolidin-2-ylidene}-acetic acid ethyl ester,
(E or Z)-Cyano-{3-ethyl-5-(E/Z)-[(4-hydroxy-3-methyl-phenylamino)-methylene]-4-oxo-thiazolidin-2-ylidene}-acetic acid ethyl ester,
(E or Z)-Cyano-{5-(E/Z)-[(3,5-dibromo-4-hydroxy-phenylamino)-methylene]-3-ethyl-4-oxo-thiazolidin-2-ylidene}-acetic acid ethyl ester,
5-{[2-((E or Z)-Cyano-ethoxycarbonyl-methylene)-3-ethyl-4-oxo-thiazolidin-5-(E/Z)-ylidenemethyl]-amino}-2-hydroxy-benzoic acid methyl ester,
(E or Z)-Cyano-{3-ethyl-5-(E/Z)-[(2-hydroxy-phenylamino)-methylene]-4-oxo-thiazolidin-2-ylidene}-acetic acid ethyl ester,
(E or Z)-Cyano-{3-ethyl-5-(E/Z)-[(2-fluoro-phenylamino)-methylene]-4-oxo-thiazolidin-2-ylidene}-acetic acid ethyl ester,
(E or Z)-Cyano-[3-ethyl-4-oxo-5-(E/Z)-(o-tolylamino-methylene)-thiazolidin-2-ylidene]-acetic acid ethyl ester,
(E or Z)-{5-(E/Z)-[(2-Chloro-phenylamino)-methylene]-3-ethyl-4-oxo-thiazolidin-2-ylidene}-cyano-acetic acid ethyl ester,
(E or Z)-Cyano-[3-ethyl-4-oxo-5-(E/Z)-(quinolin-8-ylaminomethylene)-thiazolidin-2-ylidene]-acetic acid ethyl ester,
(E or Z)-Cyano-{3-ethyl-5-(E/Z)-[(2-isopropyl-phenylamino)-methylene]-4-oxo-thiazolidin-2-ylidene}-acetic acid ethyl ester,
(E or Z)-Cyano-[3-ethyl-5-(E/Z)-(naphthalen-1-ylaminomethylene)-4-oxo-thiazolidin-2-ylidene]-acetic acid ethyl ester,
(E or Z)-Cyano-[3-ethyl-5-(E/Z)-(naphthalen-1-ylaminomethylene)-4-oxo-thiazolidin-2-ylidene]-acetic acid ethyl ester,
(E or Z)-Cyano-{3-ethyl-5-(E/Z)-[(2-ethyl-phenylamino)-methylene]-4-oxo-thiazolidin-2-ylidene}-acetic acid ethyl ester,
(E or Z)-{5-(E/Z)-[(1H-Benzoimidazol-2-ylamino)-methylene]-3-ethyl-4-oxo-thiazolidin-2-ylidene}-cyano-acetic acid ethyl ester,
(E or Z)-Cyano-{3-ethyl-5-(E/Z)-[(1-methyl-1H-benzoimidazol-2-ylamino)-methylene]-4-oxo-thiazolidin-2-ylidene}-acetic acid ethyl ester,
Cyano-[3-ethyl-4-oxo-5-[1-[4-(3-pyrrolidin-1-yl-propionylamino)-phenylamino]-meth-(E/Z)-ylidene]-thiazolidin-(2-(E or Z))-ylidene]-acetic acid allyl ester,
Cyano-[3-ethyl-4-oxo-5-[1-{4-[3-(2-pyrrolidin-1-yl-ethyl)-ureido]-phenylamino}-meth-(E/Z)-ylidene]-thiazolidin-(2-(E or Z))-ylidene]-acetic acid allyl ester,
4-(4-{[2-[1-Allyloxycarbonyl-1-cyano-meth-(E or Z)-ylidene]-3-ethyl-4-oxo-thiazolidin-5-(E/Z)-ylidenemethyl]-amino}-phenyl)-butyric acid,
Cyano-[3-ethyl-4-oxo-5-[1-[3-(3-pyrrolidin-1-yl-propionylamino)-phenylamino]-meth-(E/Z)-ylidene]-thiazolidin-(2-(E or Z))-ylidene]-acetic acid allyl ester,
4-{[2-[1-Allyloxycarbonyl-1-cyano-meth-(E or Z)-ylidene]-3-ethyl-4-oxo-thiazolidin-5-(E/Z)-ylidenemethyl]-amino}-benzoic acid,
6-{[2-[1-Allyloxycarbonyl-1-cyano-meth-(E or Z)-ylidene]-3-ethyl-4-oxo-thiazolidin-5-(E/Z)-ylidenemethyl]-amino}-naphthalene-2-carboxylic acid,
Cyano-[5-[1-{4-[3-(2-diethylamino-ethylcarbamoyl)-propyl]-phenylamino}-meth-(E/Z)-ylidene]-3-ethyl-4-oxo-thiazolidin-(2-(E or Z))-ylidene]-acetic acid allyl ester,
Cyano-[3-ethyl-4-oxo-5-[1-[6-(2-pyrrolidin-1-yl-ethylcarbamoyl)-naphthalen-2-ylamino]-meth-(E/Z)-ylidene]-thiazolidin-(2-(E or Z))-ylidene]-acetic acid allyl ester,
(E or Z)-Cyano-[3-ethyl-5-(E/Z)-({4-[3-(2-hydroxy-ethyl)-ureido]-phenylamino}-methylene)-4-oxo-thiazolidin-2-ylidene]-acetic acid ethyl ester,
(E or Z)-Cyano-[3-ethyl-4-oxo-5-(E/Z)-({4-[(pyrrolidin-1-carbonyl)-amino]-phenylamino}-methylene)-thiazolidin-2-ylidene]-acetic acid ethyl ester,
(E or Z)-Cyano-[3-ethyl-5-(E/Z)-({4-methoxy-3-[(morpholin-4-carbothioyl)-amino]-phenylamino}-methylene)-4-oxo-thiazolidin-2-ylidene]-acetic acid ethyl ester,
(E or Z)-Cyano-{3-ethyl-5-(E/Z)-[(4-{3-[2-(2-hydroxy-ethoxy)-ethyl]-ureido-phenylamino)-methylene]-4-oxo-thiazolidin-2-ylidene}-acetic acid ethyl ester,
(E or Z)-Cyano-[3-ethyl-5-(E/Z)-({4-[(4-methyl-piperazin-1-carbothioyl)-amino]-phenylamino}-methylene)-4-oxo-thiazolidin-2-ylidene]-acetic acid ethyl ester,
(E or Z)-Cyano-[3-ethyl-5-(E/Z)-({4-[3-(2-hydroxy-ethyl)-thioureido]-phenylamino}-methylene)-4-oxo-thiazolidin-2-ylidene]-acetic acid ethyl ester,
(E or Z)-{5-(E/Z)-[(4-Acetylsulfamoyl-phenylamino)-methylene]-3-ethyl-4-oxo-thiazolidin-2-ylidene}-cyanoacetic acid ethyl ester,
(E or Z)-Cyano-{3-ethyl-5-(E/Z)-[(4-{3-[2-(2-hydroxy-ethoxy)-ethyl]-thioureido}-phenylamino)-methylene]-4-oxo-thiazolidin-2-ylidene}-acetic acid ethyl ester,
(E or Z)-Cyano-(3-ethyl-5-(E/Z)-{[2-(2-hydroxy-ethyl)-phenylamino]-methylene}-4-oxo-thiazolidin-2-ylidene)-acetic acid ethyl ester,
Cyano-{3-ethyl-5-(E/Z)-[(2-ethyl-phenylamino)-methylene]-4-oxo-thiazolidin-2-ylidene}-acetic acid ethyl ester,
(E or Z)-Cyano-[3-ethyl-5-(E/Z)-({4-fluoro-3-[3-(2-morpholin-4-yl-ethyl)-ureido]-phenylamino}-methylene)-4-oxo-thiazolidin-2-ylidene]-acetic acid ethyl ester,
(E or Z)-Cyano-[3-ethyl-5-(E/Z)-({4-[3-(1-ethyl-pyrrolidin-2-ylmethyl)-ureido]-phenylamino}-methylene)-4-oxo-thiazolidin-2-ylidene]-acetic acid ethyl ester,
(E or Z)-Cyano-{3-ethyl-5-(E/Z)-[(4-{[4-(2-hydroxy-ethyl)-piperazine-1-carbonyl]-amino}-phenylamino)-methylene]-4-oxo-thiazolidin-2-ylidene}-acetic acid ethyl ester,
(E or Z)-Cyano-[3-ethyl-5-(E/Z)-({3-[3-(2-morpholin-4-yl-ethyl)-ureido]-phenylamino}-methylene)-4-oxo-thiazolidin-2-ylidene]-acetic acid ethyl ester,
(E or Z)-Cyano-[5-(E/Z)-({3-[3-(3-dimethylamino-propyl)-ureido]-phenylamino}-methylene)-3-ethyl-4-oxo-thiazolidin-2-ylidene]-acetic acid ethyl ester,
(E or Z)-Cyano-{3-ethyl-5-(E/Z)-[(4-{[4-(4-methyl-piperazin-1-yl)-piperidine-1-carbonyl]-amino}-phenylamino)-methylene]-4-oxo-thiazolidin-2-ylidene}-acetic acid ethyl ester,
(E or Z)-Cyano-[5-(E/Z)-({4-[3-(3-dimethylamino-propyl)-ureido]-phenylamino}-methylene)-3-ethyl-4-oxo-thiazolidin-2-ylidene]-acetic acid ethyl ester,
(E or Z)-Cyano-[5-(E/Z)-({3-[3-(3-dimethylamino-propyl)-ureido]-4-fluoro-phenylamino}-methylene)-3-ethyl-4-oxo-thiazolidin-2-ylidene]-acetic acid ethyl ester,
(E or Z)-Cyano-{3-ethyl-5-(E/Z)-[(4-fluoro-3-{3-[2-(1-methyl-pyrrolidin-2-yl)-ethyl]-ureido}-phenylamino)-methylene]-4-oxo-thiazolidin-2-ylidene}-acetic acid ethyl ester,
(E or Z)-Cyano-{3-ethyl-5-(E/Z)-[(4-fluoro-3-{[4-(2-hydroxy-ethyl)-piperazine-1-carbonyl]-amino}-phenylamino)-methylene]-4-oxo-thiazolidin-2-ylidene}-acetic acid ethyl ester,
(E or Z)-Cyano-[3-ethyl-4-oxo-5-(E/Z)-({4-[3-(2-pyrrolidin-1-yl-ethyl)-ureido]-phenylamino}-methylene)-thiazolidin-2-ylidene]-acetic acid ethyl ester,
(E or Z)-Cyano-[3-ethyl-5-(E/Z)-({4-[(4-methyl-piperazine-1-carbonyl)-amino]-phenylamino}-methylene)-4-oxo-thiazolidin-2-ylidene]-acetic acid ethyl ester.

9. Uses of the compounds of general formula IIA or IIB 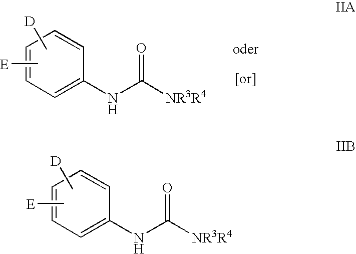
in which D stands for the group —NO₂, —NH₂ or —NH(CO)OC(CH₃)₃ and E stands for C₁-C₆-alkoxy or halogen, and R³ and R⁴ have the meaning that is described in general formula I, as intermediate products for the production of the substances of general formula I according to the invention.

10. Uses of the compounds of general formula IIIA or IIIB 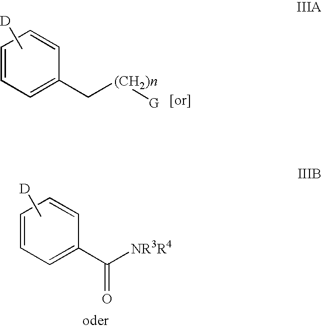
in which D stands for the group —NO₂, —NH₂ or —NH(CO)OC(CH₃)₃, and G stands for the group —NR³R⁴, and R³, R⁴ and n have the meaning that is described in general formula I, as intermediate products for the production of the substances of general formula I according to the invention.

11. Uses of the compounds of general formula IVA or IVB 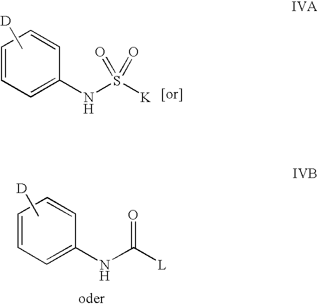
in which D stands for the group —NO₂, —NH₂ or —NH(CO)OC(CH₃)₃, and K stands for C₁-C₆-alkyl or C₁-C₆-alkenyl that is optionally substituted with the group —NR³R⁴, and L stands for C₁-C₆-alkyl or C₁-C₆-alkenyl that is optionally substituted in one or more places, in the same way or differently, with C₁-C₆-alkoxy, C₁-C₆-alkoxy-C₁-C₆-alkoxy or the group —NR³R⁴, and R³ and R⁴ have the meaning that is described in general formula I, as intermediate products for the production of substances of general formula I according to the invention.

12. Compounds of general formula V 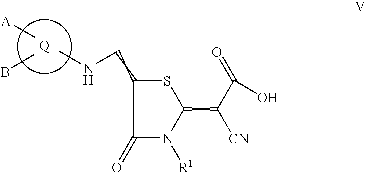
in which Q, A, B and R′ have the meaning that is described in general formula I, as intermediate products for the production of the substances of general formula I according to the invention, with the proviso that cyano-[3-ethyl-4-oxo-5-[1-phenylamino-meth-(E/Z)-ylidene]-thiazolidin-(2-(E or Z))-ylidene]-acetic acid does not fall under general formula V.

13. Use of the compounds of general formula I, according to claim 1, for the production of a pharmaceutical agent for treating cancer, auto-immune diseases, chemotherapy agent-induced alopecia and mucositis, cardiovascular diseases, infectious diseases, nephrological diseases, chronic and acute neurodegenerative diseases and viral infections.

14. Use according to claim 13, characterized in that cancer is defined as solid tumors and leukemia; auto-immune diseases are defined as psoriasis, alopecia and multiple sclerosis; cardiovascular diseases are defined as stenoses, arterioscleroses and restenoses; infectious diseases are defined as diseases that are caused by unicellular parasites; nephrological diseases are defined as glomerulonephritis; chronic neurodegenerative diseases are defined as Huntington's disease, amyotrophic lateral sclerosis, Parkinson's disease, AIDS dementia and Alzheimer's disease; acute neurodegenerative diseases are defined as ischemias of the brain and neurotraumas; and viral infections are defined as cytomegalic infections, herpes, hepatitis B and C, and HIV diseases.

15. Pharmaceutical agents that contain at least one compound according to claim 1.

16. Pharmaceutical agents according to claim 15 for treating cancer, autoimmune diseases, cardiovascular diseases, infectious diseases, nephrological diseases, neurodegenerative diseases and viral infections.

17. Compounds according to claim 1 with suitable formulation substances and vehicles.

18. Use of the compounds of general formula I, according to claim 1, as inhibitors of the polo-like kinases.

19. Use according to claim 18, wherein the kinase is Plk1, Plk2, Plk3 or Plk4.

20. Use of the compounds of general formula I, according to claim 1, in the form of a pharmaceutical preparation for enteral, parenteral and oral administration.
